(19) United States
(12) Patent Application Publication
(10) Pub. No.: US 2025/0388666 A1
(43) Pub. Date: Dec. 25, 2025

(54) HUMANIZED AND AFFINITY-MATURED ANTI-CEACAM1 ANTIBODIES AND METHODS OF USE

(71) Applicant: The Brigham and Women’s Hospital, Inc., Boston, MA (US)

(72) Inventors: Richard S. Blumberg, Weston, MA (US); Yu-Hwa Huang, Weston, MA (US); Amit Gandhi, Billerica, MA (US); Robert George Edward Holgate, Royston (GB); Arron Robert Hearn, Ely (GB); Susan Dana Jones, Boston, MA (US)

(21) Appl. No.: 18/842,534

(22) PCT Filed: Mar. 2, 2023
(86) PCT No.: PCT/US2023/063559

§ 371 Date: Aug. 29, 2024

(51) Int. Cl. C07K 16/28; A61K 40/11; A61K 40/31; A61K 40/42; A61K 45/06; A61P 35/04

(52) U.S. Cl. CPC C07K 16/2803; A61K 40/11; A61K 40/31; A61K 40/421; A61K 45/06; A61P 35/04; C07K 16/2818; A61K 2239/13; C07K 2317/31; C07K 2317/565; C07K 2317/76; C07K 2317/92

(57) ABSTRACT

Provided herein are recombinant antibodies and antigen-binding fragments thereof useful for binding to and inhibiting carcinoembryonic antigen-related cell adhesion molecule 1 (CEACAM1). Also provided are methods of using the disclosed CEACAM1 antibodies and antigen-binding fragments thereof for reducing T-cell tolerance and for the treatment of cancer and infection.

### CROSS-REFERENCE TO RELATED APPLICATION

This application claims the benefit of U.S. Provisional Patent application No. 63/316,140, filed Mar. 3, 2022, which is incorporated by reference in its entirety.

### STATEMENT REGARDING FEDERALLY SPONSORED RESEARCH OR DEVELOPMENT

This invention was made with government support under DK51362 awarded by the National Institutes of Health. The government has certain rights in the invention.

### FIELD OF THE INVENTION

The present invention relates generally to the field of molecular biology and medicine. More particularly, the invention provides monoclonal antibodies and antigen-binding fragments that bind to CEACAM1 and therapeutic compositions thereof, as well as methods of using such antibodies, including the inhibition of homophilic and heterophilic interactions with CEACAM1, and methods for treating cancer and infectious diseases.

### BACKGROUND

Carcinoembryonic antigen-related cell adhesion molecule 1 (CEACAM1) is a member of the carcinoembryonic antigen (CEA) family of immunoglobulin (Ig) like transmembrane glycoproteins. CEACAM family members are involved in cell-cell recognition and modulate cellular processes that range from the shaping of tissue architecture and neovascularization to the regulation of insulin homeostasis and T cell proliferation.

Various cellular activities have been attributed to the CEACAM1 protein, including roles in the differentiation and arrangement of tissue three-dimensional structure, angiogenesis, apoptosis, tumor suppression, metastasis, and the modulation of innate and adaptive immune responses. Further, several cell types potentially express CEACAM1, including tumor cells, T cells, B cells, natural killer (NK) cells, and certain macrophages.

High CEACAM1 expression occurs in a variety of cancers such as melanoma, colorectal, gastric, pancreatic, bladder, and thyroid cancer and is associated with worse tumor progression, metastasis, and poor clinical prognosis. Non-small cell lung cancers (NSCLC), for example, with high CEACAM1 expression exhibit high microvessel density, distant metastases, and shorter median overall survival and progression free survival. CEACAM1 expression has also been strongly correlated with distant metastasis of pancreatic adenocarcinoma. CEACAM1 expression on tumors promotes CEACAM1-mediated inhibition of T and NK cells. Consequently, inhibiting CEACAM1 activity can inhibit tumor cell metastasis and the formation of a cancer stem cell niche.

CEACAM1 is also expressed in certain immune system cells and plays a role in immune suppression and immune cell exhaustion. High CEACAM1 expression on tumor infiltrating lymphocytes (TILs) and other tumor infiltrating immune cells from gastric, lung, melanoma, colorectal cancer, and glioma, for example, is associated with a poor prognosis. On T cells, CEACAM1 expression is mostly excluded from resting (naïve) T cells, while the protein is expressed at high levels on activated T cells. CEACAM1-L is the dominant isoform expressed in most T cells and acts as an inhibitory receptor downregulating T cell activation and suppressing T cell functions. As such, inhibition of CEACAM1 on T-cells can recover T cell activity and increase anti-tumor responses.

CEACAM1 is further expressed on NK cells, which are lymphocytes involved in innate immunity, participating in early control of viral infection and immune-surveillance of tumors. When NK cells encounter cells that express major histocompatibility complex (MHC) class I, an immune response against these cells is prevented by inhibitory signals through receptor-ligand interactions. However, when encountering cells in which MHC class I is downregulated, such as in virus-infected cells or cancer cells, NK cells are activated by the lack of inhibitory signals, which makes the “diseased” cells prone to NK cell-mediated killing. When CEACAM1 is present on the surface of both NK and melanoma cells, the CEACAM1:CEACAM1 interactions lead to an inhibition of NK-mediated killing, independent of MHC class I expression. As such, disruption of this homophilic CEACAM1 interaction can be beneficial for restoring the NK-mediated immune response.

CEACAM1 expression on subsets of macrophages is further associated with fibrosis in the tumor microenvironment in mouse models. CEACAM1 also regulates other stromal cells in the tumor microenvironment such as the vascular endothelium. Therefore, inhibiting interactions of CEACAM1 with its binding partners may further inhibit fibrosis and angiogenesis.

CEACAM1 also mediates intercellular adhesion via the extracellular portion of CEACAM1 containing a IgV-like N-domain, which is involved in homophilic (CEACAM1:CEACAM1) and heterophilic interactions (e.g. with CEA, CEACAM5, CEACAM8, T cell-immunoglobulin and mucin-domain containing 3 (TIM-3) protein and Programmed Cell Death Protein 1 (PD-1), Helicobacter pylori adhesin HopQ, Neisseria gonorrhoeae/meningitidis opacity proteins (OPA), Moraxella sp. Opa-like protein OlpA, Haemophilus influenzae outer membrane protein (OMP) P1, Haemophilus aegyptius OMP P1, Fusobacterium sp., Salmonella sp., Streptococcus agalactiae, and Candida albicans. TIM-3 was identified as a Th1 specific cell surface protein that is expressed on activated T cells, subsets of dendritic cells and macrophages and NK cells. TIM-3 is an activation-induced inhibitory molecule that has been implicated in tolerance and shown to induce T cell exhaustion in chronic viral infections and cancer. CEACAM1 1, which is also expressed on activated T cells, has been shown to interact with TIM-3, and this interaction is important for TIM-3-mediated T cell inhibition. PD-1 represents a major checkpoint inhibitor pathway in humans.

As indicated above, CEACAM1 also serves as cellular receptor on the apical membrane of mucosal cells for a variety of Gram-negative bacterial pathogens associated with the human mucosa, as well as with fungal pathogens such as Candida albicans. For instance, N. gonorrhoeae, N. meningitidis, Moraxella catarrhalis, H. influenza, H. aegyptius and pathogenic Escherichia coli strains possess well-characterized CEACAM1-binding adhesins. CEACAM1 engagement with bacterial adhesins triggers endocytosis of the bacteria into epithelial cells and transcytosis of microorganisms through intact epithelial layers, thus allowing the microorganisms to exploit CEACAM1 during mucosal colonization. Additionally, CEACAM1 has been implicated in infection with influenza virus H5N1 and with filial nematodes such as Wucheria bancrofti.

### SUMMARY OF THE INVENTION

Provided herein are antibodies and antigen-binding fragments thereof that bind to CEACAM1 and that block the interaction of CEACAM1 with one or more binding partners. Also provided are therapeutic compositions of such antibodies and antigen-binding fragments thereof, as well as methods of using these antibodies. By blocking the interaction of CEACAM1 with one or more binding partners, the antibodies and antigen-binding fragments thereof are useful for reducing, inhibiting, and/or reversing T cell tolerance and/or for enhancing T cell expansion. The CEACAM1 antibodies and antigen-binding fragments thereof are further useful for treating cancer, for reducing tumor growth, for reducing tumor metastasis, and/or for reducing cancer stemness in a subject in need thereof. The CEACAM1 antibodies and antigen-binding fragments thereof are also useful for treating patients that are resistant to checkpoint therapy. Further provided are methods of using the CEACAM1 antibodies and antigen-binding fragments thereof for reducing colonization of mammalian epithelia with bacteria expressing bacterial adhesins or Candida albicans.

In one aspect, provided is an antibody or antigen-binding fragment thereof which binds to CEACAM1, the antibody or antigen-binding fragment comprising a heavy chain variable region and a light chain variable region; wherein each of the heavy chain and the light chain variable regions comprises a CDR1, CDR2, and CDR3; and wherein:
a) the sequence of CDR1H comprises sequence DYYLY (SEQ ID NO:1);
b) the sequence of CDR2H comprises sequence TISVGGGQTSYADSVKG (SEQ ID NO:2);
c) the sequence of CDR3H comprises sequence GLYYGPSWVAY (SEQ ID NO:3), ARTYGPAWFAY (SEQ ID NO:4), or ALTYGPAWLAY (SEQ ID NO:5);
d) the sequence of CDR1L comprises sequence KSSQSLLNSANQKNYLA (SEQ ID NO:6);
e) the sequence of CDR2L comprises sequence FASTRES (SEQ ID NO:7); and
f) the sequence of CDR3L comprises sequence QSHYPFYYT (SEQ ID NO:8) or QSHFPYPLT (SEQ ID NO:9).

In an embodiment, the sequence of CDR3H comprises sequence GLYYGPSWVAY (SEQ ID NO:3); and the sequence of CDR3L comprises sequence QSHYPFYYT (SEQ ID NO:8).

In one aspect, provided is an antibody or antigen-binding fragment thereof which binds to CEACAM1, the antibody or antigen-binding fragment comprising a heavy chain variable region and a light chain variable region, wherein the sequence of the heavy chain variable region comprises a sequence that is at least 90% identical to a sequence selected from the group consisting of SEQ ID NOs:15-17 and wherein the sequence of the light chain variable region comprises a sequence that is at least 90% identical to SEQ ID NO:21 or SEQ ID NO:22.

In an embodiment, the antibody or antigen-binding fragment thereof comprises a heavy chain variable region comprising a sequence that is at least 90% identical to a heavy chain variable region amino acid sequence of SEQ ID NO:15 and a light chain variable region comprising a sequence that is at least 90% identical to a light chain variable region amino acid sequence of any one of SEQ ID NO:21.

In embodiments, the heavy chain variable region comprises a sequence that is at least 95% identical to a sequence selected from the group consisting of SEQ ID NOs:15-17 and the light chain variable region comprises a sequence that is at least 95% identical to SEQ ID NO:21 or SEQ ID NO:22. In an embodiment, the sequence of the heavy chain variable region comprises a sequence that is at least 95% identical to a heavy chain variable region amino acid sequence of SEQ ID NO:15 and the sequence of the light chain variable region comprises a sequence that is at least 95% identical to a light chain variable region amino acid sequence of any one of SEQ ID NO:21.

In embodiments, the sequence of the heavy chain variable region comprises a sequence selected from the group consisting of SEQ ID NOs:15-17 and the sequence of the light chain variable region comprises SEQ ID NO:21 or SEQ ID NO:22. In one embodiment, the sequence of the heavy chain variable region comprises SEQ ID NO:15 and the sequence of the light chain variable region comprises SEQ ID NO:21.

In some embodiments, the antibody or antigen-binding fragment thereof is a chimeric antibody, a CDR-grafted antibody, or a humanized antibody or antigen-binding fragment thereof.

In some embodiments, the antibody or antigen-binding fragment is a multispecific or a bispecific antibody or antigen-binding fragment thereof. In some embodiments, the antibody or antigen-binding fragment is a bispecific antibody comprising a complementary region that binds to PD-1, PD-L1, CTLA-4, TIM-3, epidermal growth factor receptor (EGFR), CD25, CD19 or Fc gamma receptor.

In some embodiments, the antibody or antigen-binding fragment is an scFv, Fv, Fab′, Fab, F(ab′)₂, or diabody.

In some embodiments, the antibody or antigen-binding fragment has isotype IgG4.

In some embodiments, the antibody or antigen-binding fragment thereof contains a S241P substitution in the constant region of the heavy chain.

In some embodiments, the antibody or antigen-binding fragment is deglycosylated.

In some embodiments, the antibody or antigen-binding fragment is lacking a C-terminal lysine in the heavy chain.

In some embodiments, the antibody or antigen-binding fragment is conjugated to one or more of a cytotoxin, a fluorescent label and an imaging agent.

Provided herein is an antibody or antigen-binding fragment thereof that binds to the same epitope on CEACAM1 as an antibody or antigen-binding fragment thereof disclosed herein. Provided herein is an antibody or antigen-binding fragment thereof that binds to the IgV-like N-domain domain of CEACAM1. In embodiments, the antibody or antigen-binding fragment thereof does not bind to one of more of CEACAM3, CEACAM5, CEACAM6, and CEACAM 8. In some embodiments, the antibody or antigen-binding fragment thereof at least partially binds to the binding site on CEACAM1 for TIM-3 and/or at least partially binds to the binding site on CEACAM1 for PD-1. In some embodiments, the antibody or antigen-binding fragment at least partially binds to the binding site on CEACAM1 for CEACAM1 during homo-dimerization.

Provided herein is a nucleic acid encoding an antibody or antigen-binding fragment thereof disclosed herein. In one embodiment, the nucleic acid is an isolated nucleic acid. Provided herein is a vector comprising a nucleic acid disclosed herein.

Provided herein is a cell comprising a vector disclosed herein. In one embodiment, the cell is an isolated cell. Provided herein is a cell expressing an antibody or antigen-binding fragment thereof disclosed herein. Provided herein is a T-cell with a chimeric antigen receptor comprising the CDRs of an antibody or antigen-binding fragment thereof disclosed herein.

Provided herein is a pharmaceutical composition comprising an antibody or antigen-binding fragment thereof disclosed herein and a pharmaceutically acceptable excipient.

Provided herein is a method of inhibiting binding of CEACAM1 to a member of the CEACAM family, the method comprising contacting CEACAM1 with an antibody or antigen-binding fragment thereof disclosed herein or a pharmaceutical composition disclosed herein. In some embodiments, the member of the CEACAM family is CEACAM5 or CEACAM8. In one embodiment, the member of the CEACAM family is CEACAM1.

Provided is a method of inhibiting binding of CEACAM1 to a member of the TIM family, the method comprising contacting CEACAM1 with an antibody or antigen-binding fragment thereof disclosed herein or a pharmaceutical composition disclosed herein. In one embodiment, the member of the TIM family is TIM-3.

Provided is a method of inhibiting binding of CEACAM1 to PD-1, the method comprising contacting CEACAM1 with an antibody or antigen-binding fragment thereof disclosed herein or a pharmaceutical composition disclosed herein.

A method of inhibiting binding of CEACAM1 to a bacterial adhesion, the method comprising contacting CEACAM1 with an antibody or antigen-binding fragment thereof disclosed herein or a pharmaceutical composition disclosed herein. In embodiments, the bacterial adhesin is Helicobacter pylori adhesin HopQ, Neisseria gonorrhoeae opacity protein (Opa), Neisseria meningitidis Opa, Haemophilus influenza outer membrane protein (OMP) P1, Haemophilus aegyptius OMP P1, Moraxella sp. Opa-like protein (OlpA), a Fusobacterium sp. trimeric autotransporter adhesin CbpF, a Salmonella sp. adhesin, or a Streptococcus agalactiae IgI3-like β protein adhesin.

Provided is a method of inhibiting binding of CEACAM1 to a Candida albicans, the method comprising contacting CEACAM1 with an antibody or antigen-binding fragment thereof disclosed herein or a pharmaceutical composition disclosed herein.

Provided is a method of reducing colonization of mammalian epithelia with bacteria expressing bacterial adhesins, the method comprising contacting CEACAM1 with an antibody or antigen-binding fragment thereof disclosed herein or a pharmaceutical composition disclosed herein. In embodiments, the bacterial adhesin is Helicobacter pylori adhesin HopQ, Neisseria gonorrhoeae opacity protein (Opa), Neisseria meningitidis Opa, Haemophilus influenza OMP P1, Haemophilus aegyptius OMP P1 or Moraxella sp. OlpA, a Fusobacterium sp. trimeric autotransporter adhesin CbpF, a Salmonella sp. adhesin, or a Streptococcus agalactiae IgI3-like β protein adhesin.

Provided herein is a method of reducing colonization of mammalian epithelia with Candida albicans, the method comprising contacting CEACAM1 with an antibody or antigen-binding fragment thereof disclosed herein or a pharmaceutical composition disclosed herein.

Provided herein is a method of reducing T cell tolerance, the method comprising contacting a cell population comprising T cells with an antibody or antigen-binding fragment thereof disclosed herein or a pharmaceutical composition disclosed herein.

Provided herein is a method of enhancing T cell expansion, the method comprising contacting a cell population comprising T cells with an antibody or antigen-binding fragment thereof disclosed herein or a pharmaceutical composition disclosed herein.

Provided herein is a method of reducing T cell tolerance in a subject in need thereof, the method comprising administering to the subject an antibody or antigen-binding fragment thereof disclosed herein or a pharmaceutical composition disclosed herein.

Provided herein is a method of enhancing T cell expansion in a subject in need thereof, the method comprising administering to the subject an antibody or antigen-binding fragment thereof disclosed herein or a pharmaceutical composition disclosed herein.

Provided herein is a method of treating cancer in a subject in need thereof, the method comprising administering to the subject an antibody or antigen-binding fragment thereof disclosed herein or a pharmaceutical composition disclosed herein. In embodiments, the cancer is melanoma, pancreatic cancer, thyroid cancer, lung cancer, colorectal cancer, squamous cancer, prostate cancer, breast cancer, bladder cancer, or gastric cancer.

Provided herein is a method of reducing tumor growth in a subject in need thereof, the method comprising administering to the subject an antibody or antigen-binding fragment thereof disclosed herein or a pharmaceutical composition disclosed herein.

Provided herein is a method of reducing tumor metastasis in a subject in need thereof, the method comprising administering to the subject an antibody or antigen-binding fragment thereof disclosed herein or a pharmaceutical composition disclosed herein.

Provided herein is a method of reducing tumor-associated fibrosis in a subject in need thereof, the method comprising administering to the subject an antibody or antigen-binding fragment thereof disclosed herein or a pharmaceutical composition disclosed herein n.

Provided herein is a method of reducing cancer sternness in a subject in need thereof, the method comprising administering to the subject an antibody or antigen-binding fragment thereof disclosed herein or a pharmaceutical composition disclosed herein.

Provided herein is a method of reducing colonization of a subject's epithelia with bacteria expressing bacterial adhesins in a subject in need thereof, the method comprising administering to the subject an antibody or antigen-binding fragment thereof disclosed herein. In embodiments, the bacterial adhesin is Helicobacter pylori adhesin HopQ, Neisseria gonorrhoeae opacity protein (Opa), Neisseria meningitidis Opa, Haemophilus influenza OMP P1, Haemophilus aegyptius OMP P1, Moraxella sp. OlpA, a Fusobacterium sp. trimeric autotransporter adhesin CbpF, a Salmonella sp. adhesin, or a Streptococcus agalactiae IgI3-like β protein adhesin.

Provided herein is a method of reducing colonization of a subject's epithelia with Candida albicans in a subject in need thereof, the method comprising administering to the subject an antibody or antigen-binding fragment thereof disclosed herein or a pharmaceutical composition disclosed herein.

Provided herein is a method of reducing invasion of a subject's lymphatic system with a filarial worm in a subject in need thereof, the method comprising administering to the subject an antibody or antigen-binding fragment thereof disclosed herein. In one embodiment, the filarial worm is Wucheria bancrofti.

Provided herein is a method of reducing the invasion of a subject's lymphatic system with cancer cells in a subject in need thereof, the method comprising administering to the subject an antibody or antigen-binding fragment thereof disclosed herein or a pharmaceutical composition disclosed herein. In embodiments, the method further comprising administering a checkpoint inhibitor. In embodiments, the checkpoint inhibitor is a CTLA-4, a PD-1, a PD-L1, and a PD-L2 inhibitor.

In embodiments, a method disclosed herein further comprises administering one or more of an inhibitor of LAG3, TIGIT, LAP, Podoplanin, Protein C receptor, ICOS, GITR, CD226 and/or CD160. In embodiments, a method disclosed herein further comprises administering a TIM-3 inhibitor. In embodiments, the additional inhibitor is administered concurrently or consecutively with the antibody or antigen-binding fragment or the pharmaceutical composition. In embodiments, the additional inhibitor is administered separately or as a mixture with an antibody or antigen-binding fragment thereof disclosed herein.

Provided is a method of treating a subject in need thereof, the method comprising administering to the subject an antibody or antigen-binding fragment thereof disclosed herein or a pharmaceutical composition disclosed herein, wherein the subject has acquired resistance to therapy with a checkpoint inhibitor therapy. In embodiments, the subject has acquired resistance to therapy with one or more of a PD-1 inhibitor, a PD-L1 inhibitor, or a CTLA-4 inhibitor.

### BRIEF DESCRIPTION OF THE DRAWINGS

FIG. 1 illustrates the affinity maturation library design. CDRs (as defined by Kabat) are underlined. X=Positions targeted for mutagenesis. Individual positions may contain all 20 amino acids or a subset thereof. VH CDR3 block for mutagenesis CTRGLYYGPAWFAYW (SEQ ID NO:25). VL CDR 3 block for mutagenesis CQQHYSTPWTF (SEQ ID NO:26).

FIG. 2 illustrates exemplary data from the periprep binding primary screen. The parent humanized scFv, an irrelevant scFv, and the media only wells are indicated. Clones with at least 50% inhibition are indicated with an arrow.

FIGS. 3A, 3B, 3C, 3D, and 3E show results from cross reactivity ELISA of the parent antibody, twelve lead variants (FIGS. 3A, 3B, 3C, and 3D), and pan CEACAM antibody D14HDT1 (FIG. 3E) binding to CEACAM-1/-3/-5 and -6 (from left to right) at three concentrations (1.67 μg/ml, 0.062 μg/ml and 0.0069 μg/ml).

FIGS. 4A and 4B illustrate the specificity of antibody He14/L1 as determined by single cycle surface plasmon resonance. Binding sensorgrams are shown for increasing concentrations (70 to 280 nM) of antibody He14/L1 (FIG. 4A) or control antibody CH14D11 (FIG. 4B) binding to hCEACAM1, hCEACAM3, hCEACAM5, and hCEACAM6. RU, Resonance units.

FIGS. 5A, 5B, 5C, 5D, and 5E show the structure of the CEACAM1:He14/L1 Fab complex. FIG. 5A. Structure of the complex (L, H, and C chains) with the Fab heavy and light chains drawn as a Ca traces and the antigen drawn as a ribbon. FIG. 5B. The molecule of CEACAM1 in the complex is superimposed upon a molecule of CEACAM1 in the dimer. Strands are labeled A to G in bold. FIG. 5C. A view of complexed CEACAM1 showing the epitope. The molecular surface drawn is drawn in semi-transparent light gray, the protein backbone is drawn as a ribbon, and relevant side chains are drawn as sticks and labeled. Areas of the surface interacting with the Fab are indicated. FIG. 5D. Overall Binding He14/L1 with CEACAM1 GFCC′ face. FIG. 5E. He14L1 specifically docks on both faces of CEACAM1 and may disrupt higher order CEACAM1 structures GFCC′ face is primary interaction face in addition to C′C″DE face potentially associated with higher order structures.

FIG. 6 illustrates that the indicated CEACAM1 antibodies VH2/Vκ4 (parental/parental), VH2/L1 (parental/L1), He3/L20, He17/L1, and He14/L1 block human CEACAM: human TIM-3 interactions. IgG4=control antibody.

FIG. 7 shows the results of an in vitro assay of T cell function (proliferation assay). Ki67 expression indicates T cell proliferation in response to CEACAM1 antibodies He3/L20, He14/L1, H17/L1, and VH2/L1 (parental/L1) in a subject resistant to pembrolizumab treatment.

FIGS. 8A and 8B show that treatment with antibody He14/L1 (FIG. 8A) resulted in the expansion of B cells (from 164 to 347 cells) and T cells (from 394 to 913 cells) within 42 hours as compared to the control antibody (FIG. 8B), indicating that He14/L1 causes broad induction of B and T cells, responses that are important to anti-tumor activity.

FIGS. 9A, 9B, and 9C illustrate that CEACAM1 expression of the epitope recognized by antibodies derived from the parental antibody including He14/L1 is associated with treatment resistance. FIG. 9A. CEACAM1 expression for metaclusters representing pre-plasmablastic B cells identified by Citrus analysis (metaclusters identified by Citrus C36175, C36194) shows that treatment-resistance is associated with increased CEACAM1 expression for resistant (R) compared to naive (N) tumor samples. Median levels of CEACAM1 expression determined by ANOVA. *, p<0.05. FIG. 9B. CEACAM1 expression for metaclusters representing tumor-associated monocytic cells shows that treatment-resistance is associated with increased CEACAM1 expression for resistant (R) compared to naive (N) tumor samples in metaclusters identified by Citrus (C58750, C58749, C58832, C58843, C58885, C58888). Median levels of CEACAM1 expression determined by ANOVA. *, p<0.05 and **, p<0.01 significance. FIG. 9C. CEACAM1 expression for metaclusters representing dysfunctional CD8⁺ central memory T cells shows that treatment-resistance is associated with increased CEACAM1 expression for resistant (R) compared to naive (N) tumor samples in metaclusters identified by Citrus (C58736, C58786, C58789). Median levels of CEACAM1 expression determined by ANOVA. *, p<0.05 significance.

### DETAILED DESCRIPTION OF THE INVENTION

### Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies), antibody fragments, and antigen-binding portions thereof (e.g., paratopes, CDRs), so long as they exhibit the desired biological activity and specificity.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. VL refers to the variable domain of the light chain. The amino acid positions assigned to CDRs and FRs may be defined according to Kabat or according to Chothia. The term “framework regions” (FR) refers to those variable domain residues other than the CDR residues.

As used herein, the term “Complementarity Determining Regions” (CDRs) refers to portions of an antibody variable domain that are (typically) involved in antigen binding. Each variable domain typically has three CDR regions identified as CDR1, CDR2 and CDR3. Each CDR can comprise amino acid residues from a CDR as defined by e.g., Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1987, 1991)). Each CDR can also comprise amino acid residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain (Chothia & Lesk 196 J. Mol. Biol. 901 (1987)). In some instances, a CDR can include amino acids from both a CDR region defined according to Kabat and a hypervariable loop. The Kabat residue designations do not always correspond directly with the linear numbering of the amino acid residues (primary amino acid sequence). The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues may be determined for a given antibody or antigen-binding fragment thereof by alignment of residues of homology in the sequence of the antibody or antigen-binding fragment thereof with a “standard” Kabat numbered sequence. Alternatively, a CDR can be defined according to the ImMunoGeneTics (IMGT) system (Lefranc, M.-P. et al., Dev. Comp. Immunol., 27, 55-77 (2003)).

In one embodiment, the CEACAM1 antibody or antigen-binding fragment thereof provided herein comprises a heavy variable chain comprising three CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2);
(iii) the sequence of CDR3H comprises sequence GLYYGPSWVAY (SEQ ID NO:3), ARTYGPAWFAY (SEQ ID NO:4), or ALTYGPAWLAY (SEQ ID NO:5).

In one embodiment, the CEACAM1 antibody or antigen-binding fragment thereof provided herein comprises a heavy variable chain comprising three CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2); and
(iii) the sequence of CDR3H comprises sequence GLYYGPSWVAY (SEQ ID NO:3).

In one embodiment, the CEACAM1 antibody or antigen-binding fragment thereof provided herein comprises a heavy variable chain comprising three CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2); and
(iii) the sequence of CDR3H comprises sequence ARTYGPAWFAY (SEQ ID NO:4).

In one embodiment, the CEACAM1 antibody or antigen-binding fragment thereof provided herein comprises a heavy variable chain comprising three CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2); and
(iii) the sequence of CDR3H comprises sequence ALTYGPAWLAY (SEQ ID NO:5).

In one embodiment, the CEACAM1 antibody or antigen-binding fragment thereof provided herein comprises a light variable chain comprising three CDRs, wherein:
(i) the sequence of CDR1L comprises sequence KSSQSLLNSANQKNYLA (SEQ ID NO:6);
(ii) the sequence of CDR2L comprises sequence FASTRES (SEQ ID NO:7); and
(iii) the sequence of CDR3L comprises sequence QSHYPFYYT (SEQ ID NO:8) or QSHFPYPLT (SEQ ID NO:9).

In one embodiment, the CEACAM1 antibody or antigen-binding fragment thereof provided herein comprises a light variable chain comprising three CDRs, wherein:
(i) the sequence of CDR1L comprises sequence KSSQSLLNSANQKNYLA (SEQ ID NO:6);
(ii) the sequence of CDR2L comprises sequence FASTRES (SEQ ID NO:7); and
(iii) the sequence of CDR3L comprises sequence QSHYPFYYT (SEQ ID NO:8).

In one embodiment, the CEACAM1 antibody or antigen-binding fragment thereof provided herein comprises a light variable chain comprising three CDRs, wherein:
(i) the sequence of CDR1L comprises sequence KSSQSLLNSANQKNYLA (SEQ ID NO:6);
(ii) the sequence of CDR2L comprises sequence FASTRES (SEQ ID NO:7); and
(iii) the sequence of CDR3L comprises sequence QSHFPYPLT (SEQ ID NO:9).

In one embodiment, the CEACAM1 antibody or antigen-binding fragment thereof provided herein comprises six CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2);
(iii) the sequence of CDR3H comprises sequence GLYYGPSWVAY (SEQ ID NO:3), ARTYGPAWFAY (SEQ ID NO:4), or ALTYGPAWLAY (SEQ ID NO:5);
(iv) the sequence of CDR1L comprises sequence KSSQSLLNSANQKNYLA (SEQ ID NO:6);
(v) the sequence of CDR2L comprises sequence FASTRES (SEQ ID NO:7); and
(vi) the sequence of CDR3L comprises sequence QSHYPFYYT (SEQ ID NO:8) or QSHFPYPLT (SEQ ID NO:9).

In one embodiment, the CEACAM1 antibody or antigen-binding fragment thereof provided herein comprises six CDRs, wherein:
(i) the sequence of CDR1H comprises sequence DYYLY (SEQ ID NO:1);
(ii) the sequence of CDR2H comprises sequence TISVGGGQTSYADSVKG (SEQ ID NO:2);
(iii) the sequence of CDR3H comprises sequence GLYYGPSWVAY (SEQ ID NO:3);
(iv) the sequence of CDR1L comprises sequence KSSQSLLNSANQKNYLA (SEQ ID NO:6);
(v) the sequence of CDR2L comprises sequence FASTRES (SEQ ID NO:7); and
(vi) the sequence of CDR3L comprises sequence QSHYPFYYT (SEQ ID NO:8).

According to certain embodiments, the contemplated antibodies and antigen-binding fragments thereof also feature humanized frameworks for reduced immunogenicity. In certain embodiments, the CDRs of the contemplated antibody or antigen-binding fragment thereof are located in frameworks obtained from a human antibody or antigen-binding fragment thereof. In other embodiments, surface-exposed framework residues of the contemplated antibody or antigen-binding fragment thereof are replaced with framework residues of a human antibody or antigen-binding fragment thereof. The CDRs may also be located in murine or humanized frameworks linked to human constant regions (i.e., chimeric antibodies). In a preferred embodiment, the CDRs of a contemplated antibody or antigen-binding fragment thereof are located in frameworks that are a composite of two or more human antibodies. In such embodiments, the contemplated antibodies or antigen-binding fragments thereof comprise two or more sequence segments (“composites”) derived from V-regions of unrelated human antibodies that are selected to maintain monoclonal antibody sequences important for antigen binding of the starting precursor anti-human CEACAM1 monoclonal antibody, and which have all been filtered for the presence of potential T cell epitopes using “in silico tools” (Holgate & Baker, IDrugs. 2009 April; 12(4):233-7). The close fit of human sequence segments with all sections of the starting antibody V regions and the elimination of CD4⁺ T cell epitopes prior to synthesis of the antibody or antigen-binding fragment thereof allow this technology to circumvent immunogenicity while maintaining optimal affinity and specificity through the prior analysis of sequences necessary for antigen-specificity (Holgate & Baker, 2009).

Also provided herein variable heavy chain and variable light chain sequences as well as pairing thereof that comprises sequences that are similar, but not identical to the variable heavy chain and variable light chains disclosed in SEQ ID NOs:10-22 and pairings thereof.

In one embodiment, the CEACAM1 antibody or antigen-binding fragment thereof comprises a heavy chain variable domain comprising a sequence that is at least 80%, at least 85%, at least 90%, at least 95%, at least 96%, at least 97%, at least 98%, or at least 99% identical to a sequence selected from the group consisting of SEQ ID NOs:10-17. In one embodiment, the CEACAM1 antibody or antigen-binding fragment thereof comprises a heavy chain variable domain comprising a sequence that is at least 80%, at least 85%, at least 90%, at least 95%, at least 96%, at least 97%, at least 98%, or at least 99% identical to a sequence selected from the group consisting of SEQ ID NOs:15-17.

In some embodiments, the CEACAM1 antibody or antigen-binding fragment thereof comprises a variable heavy chain amino acid sequence selected from the group consisting of SEQ ID NOs:10-17. In some embodiments, the CEACAM1 antibody or antigen-binding fragment thereof comprises a variable heavy chain amino acid sequence selected from the group consisting of SEQ ID NOs:15-17.

In one embodiment, the CEACAM1 antibody or antigen-binding fragment thereof comprises a light chain variable domain comprising a sequence that is at least 80%, at least 85%, at least 90%, at least 95%, at least 96%, at least 97%, at least 98%, or at least 99% identical to a sequence selected from the group consisting of SEQ ID NOs:18-22.

In one embodiment, the CEACAM1 antibody or antigen-binding fragment thereof comprises a light chain variable domain comprising a sequence that is at least 80%, at least 85%, at least 90%, at least 95%, at least 96%, at least 97%, at least 98%, or at least 99% identical SEQ ID NO:21 or SEQ ID NO:22.

In some embodiments, the antibody or antigen-binding fragment thereof comprises a variable light chain amino acid sequence selected from the group consisting of SEQ ID NOs:18-22. In some embodiments, the antibody or antigen-binding fragment thereof comprises a variable light chain amino acid sequence comprising SEQ ID NO:21 or SEQ ID NO:22.

In one embodiment, the CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a sequence selected from the group consisting of SEQ ID NOs:10-17; and
(ii) a light chain variable domain comprising a sequence that is at least 80%, at least 85%, at least 90%, at least 95%, at least 96%, at least 97%, at least 98%, or at least 99% identical to a sequence selected from the group consisting of SEQ ID NOs:18-22.

In one embodiment, the CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a sequence selected from the group consisting of SEQ ID NOs:15-17; and
(ii) a light chain variable domain comprising a sequence that is at least 80%, at least 85%, at least 90%, at least 95%, at least 96%, at least 97%, at least 98%, or at least 99% identical to SEQ ID NO:21 or SEQ ID NO:22.

In one embodiment, the CEACAM1 antibody or antigen-binding fragment thereof comprises
(i) a heavy chain variable domain comprising a sequence selected from the group consisting of SEQ ID NOs:10-17; and/or
(ii) a light chain variable domain comprising a sequence selected from the group consisting of SEQ ID NOs:18-22.

In one embodiment, the CEACAM1 antibody or antigen-binding fragment thereof comprises
(i) a heavy chain variable domain comprising a sequence selected from the group consisting of SEQ ID NOs:15-17; and/or
(ii) a light chain variable domain comprising SEQ ID NO:21 or SEQ ID NO:22.

In one embodiment, the CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to SEQ ID NO:15; and
(ii) a light chain variable domain comprising a sequence that is at least 80%, at least 85%, at least 90%, at least 95%, at least 96%, at least 97%, at least 98%, or at least 99% identical to SEQ ID NO:21.

In one embodiment, the CEACAM1 antibody or antigen-binding fragment thereof comprises
(i) a heavy chain variable domain comprising SEQ ID NO:15; and
(ii) a light chain variable domain comprising SEQ ID NO:21.

In one embodiment, the CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to SEQ ID NO:16; and
(ii) a light chain variable domain comprising a sequence that is at least 80%, at least 85%, at least 90%, at least 95%, at least 96%, at least 97%, at least 98%, or at least 99% identical to SEQ ID NO:21.

In one embodiment, the CEACAM1 antibody or antigen-binding fragment thereof comprises
(i) a heavy chain variable domain comprising SEQ ID NO:16; and
(ii) a light chain variable domain comprising SEQ ID NO:21.

In one embodiment, the CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to SEQ ID NO:17; and
(ii) a light chain variable domain comprising a sequence that is at least 80%, at least 85%, at least 90%, at least 95%, at least 96%, at least 97%, at least 98%, or at least 99% identical to SEQ ID NO:22.

In one embodiment, the CEACAM1 antibody or antigen-binding fragment thereof comprises
(i) a heavy chain variable domain comprising SEQ ID NO:17; and
(ii) a light chain variable domain comprising SEQ ID NO:22.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In one embodiment, provided is an CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:15;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:21; and
(iii) six CDRs, wherein:
   a. the sequence of CDR1 of the heavy chain variable region comprises SEQ ID NO: 1;
   b. the sequence of CDR2 of the heavy chain variable region comprises SEQ ID NO:2;
   c. the sequence of CDR3 of the heavy chain variable region comprises SEQ ID NO: 3;
   d. the sequence of CDR1 of the light chain variable region comprises SEQ ID NO:6;
   e. the sequence of CDR2 of the light chain variable region comprises SEQ ID NO:7; and
   f. the sequence of CDR3 of the light chain variable region comprises SEQ ID NO:8.

In one embodiment, provided is an CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:16;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:21; and
(iii) six CDRs, wherein:
   a. the sequence of CDR1 of the heavy chain variable region comprises SEQ ID NO: 1;
   b. the sequence of CDR2 of the heavy chain variable region comprises SEQ ID NO:2;
   c. the sequence of CDR3 of the heavy chain variable region comprises SEQ ID NO:4;
   d. the sequence of CDR1 of the light chain variable region comprises SEQ ID NO:6;
   e. the sequence of CDR2 of the light chain variable region comprises SEQ ID NO:7; and
   f. the sequence of CDR3 of the light chain variable region comprises SEQ ID NO:8.

In one embodiment, provided is an CEACAM1 antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:17;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:22; and
(iii) six CDRs, wherein:
   a. the sequence of CDR1 of the heavy chain variable region comprises SEQ ID NO: 1;
   b. the sequence of CDR2 of the heavy chain variable region comprises SEQ ID NO:2;
   c. the sequence of CDR3 of the heavy chain variable region comprises SEQ ID NO: 5;
   d. the sequence of CDR1 of the light chain variable region comprises SEQ ID NO:6;
   e. the sequence of CDR2 of the light chain variable region comprises SEQ ID NO: 7; and
   f. the sequence of CDR3 of the light chain variable region comprises SEQ ID NO:9.

It will be evident that any of the frameworks described herein can be utilized in combination with any of the CDRs and CDR motifs described herein. In some embodiments, the CEACAM1 antibody or antigen-binding fragment thereof utilizes a framework described in Table 4.

In some embodiments of the aspects described herein, amino acid sequence modification(s) of the antibodies or antigen-binding fragments thereof that bind to CEACAM1 described herein are contemplated. Amino acid sequence variants of the antibody or antigen-binding fragment thereof are prepared by introducing appropriate nucleotide changes into the nucleic acid encoding the antibody or antigen-binding fragment thereof, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, provided that the final construct possesses the desired characteristics, e.g., binding specificity, inhibition of biological activity.

One type of variant is a conservative amino acid substitution variant. These variants have at least one amino acid residue in the antibody or antigen-binding fragment thereof replaced by a different residue that has similar side chain properties. Amino acids can be grouped according to similarities in the properties of their side chains (see Lehninger, BIOCHEMISTRY (2nd ed., Worth Publishers, New York, 1975): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser (S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).
   As such, a non-limiting example for a conservative amino acid substitution is one that replaces a hydrophobic amino acid with another hydrophobic amino acid.

Further contemplated are amino acid sequence insertions, which can include amino- and/or carboxyl-terminal fusions ranging in length from one residue to polypeptides containing a hundred or more residues, as well as intrasequence insertions of single or multiple amino acid residues. Examples of terminal insertions include an antibody or antigen-binding fragment thereof with an N-terminal methionyl residue or the antibody or antigen-binding fragment thereof fused to a cytotoxic polypeptide. Other insertional variants of the antibody or antigen-binding fragment thereof include the fusion to the N- or C-terminus of the antibody or antigen-binding fragment thereof to an enzyme or a polypeptide which increases the serum half-life of the antibody or antigen-binding fragment thereof, such as, for example, biotin.

Any cysteine residue not involved in maintaining the proper conformation of the antibodies or antigen-binding fragments thereof that bind to CEACAM1 also can be substituted, for example with a serine or an alanine, to improve the oxidative stability of the molecule and prevent aberrant crosslinking.

Conversely, cysteine bond(s) can be added to the antibody or antigen-binding fragment thereof to improve its stability (particularly where the antibody or antigen-binding fragment thereof is an antibody fragment such as an Fv fragment).

In some embodiments, the antibodies or antigen-binding fragments thereof describes have amino acid alterations that alter the original glycosylation pattern of the antibody or antigen-binding fragment thereof. By “altering the original glycosylation pattern” is meant deleting one or more carbohydrate moieties found in the antibody or antigen-binding fragment thereof, and/or adding one or more glycosylation sites that are not present in the antibody or antigen-binding fragment thereof. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. Addition of glycosylation sites to the antibodies or antigen-binding fragments thereof that bind to CEACAM1 is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody or antigen-binding fragment thereof (for O-linked glycosylation sites).

In some embodiments, the CEACAM1 antibodies or antigen-binding fragments thereof provided herein are deglycosylated or aglycosylated. In some embodiments, the contemplated CEACAM1 antibody or antigen-binding fragment thereof lacks a C-terminal lysine in the heavy chain and/or contains a S241P substitution in the constant region of the heavy chain. In some embodiments, the CEACAM1 antibody or antigen-binding fragment thereof lacks a glycosylation site in the CDR1 of the variable light chain. In some embodiments, the CEACAM1 antibody or antigen-binding fragment thereof lacks an N-X-S/T consensus sequence in the CDR1 of the variable light chain. In some embodiments, the CEACAM1 antibody or antigen-binding fragment thereof has a mutation in CDR residues 26 and/or 29 (Kabat numbering) of the CDR1 of the variable light chain. Where the antibody or antigen-binding fragment thereof comprises an Fc region, the carbohydrate(s) attached thereto can be altered. For example, antibodies with a mature carbohydrate structure that lacks fucose attached to an Fc region of the antibody or antigen-binding fragment thereof are described. See, e.g., U.S. Patent Pubs. No. 2003/0157108; No. 2004/0093621. Antibodies with a bisecting N-acetylglucosamine (GlcNAc) in the carbohydrate attached to an Fc region of the antibody or antigen-binding fragment thereof are referenced in WO 03/011878; U.S. Pat. No. 6,602,684. Antibodies with at least one galactose residue in the oligosaccharide attached to an Fc region of the antibody or antigen-binding fragment thereof are reported in WO 97/30087. See also WO 98/58964; WO 99/22764 concerning antibodies with altered carbohydrate attached to the Fe region thereof.

In some embodiments, it can be desirable to modify the antibodies or antigen-binding fragment thereof that bind to CEACAM1 described herein with respect to effector function, e.g., so as to enhance antigen-dependent cell-mediated cytotoxicity (ADCC) and/or complement dependent cytotoxicity (CDC) of the antibody or antigen-binding fragment thereof. This can be achieved by introducing one or more amino acid substitutions in an Fc region of the antibody or antigen-binding fragment thereof. Alternatively, or additionally, one or more cysteine residues can be introduced in the Fc region, thereby allowing interchain disulfide bond formation in this region. The homodimeric antibody or antigen-binding fragment thereof thus generated can have improved internalization capability and/or increased complement-mediated cell killing and antibody-dependent cellular cytotoxicity (ADCC). See Caron et al., 176 J. Exp. Med. 1191 (1992); Shopes, 148 J. Immunol. 2918 (1992). Homodimeric antibodies with enhanced anti-tumor activity can also be prepared using heterobifunctional cross-linkers as described in Wolff et al., 53 Cancer Res. 2560 (1993). Alternatively, an antibody or antigen-binding fragment thereof can be engineered which has dual Fc regions and can thereby have enhanced complement lysis and ADCC capabilities. See Stevenson et al., 3 Anti-Cancer Drug Design 219 (1989).

In some embodiments, the antibodies or antigen-binding fragments thereof disclosed herein are modified to exhibit effector function reduction or elimination. This can, for example, be accomplished by: (i) reduction or elimination of wild-type mammalian glycosylation of the antibody, (for example, by producing the antibody in an environment where such glycosylation cannot occur, by mutating one or more carbohydrate attachment points such that the antibody cannot be glycosylated, or by chemically or enzymatically removing one or more carbohydrates from the antibody after it has been glycosylated); (ii) by reduction or elimination of the Fc receptor-binding capability of the antibody (for example, by mutation of the Fc region, by deletion within the Fc region or elimination of the Fc region); or (iii) by utilization of an antibody isotype known to have minimal or no effector function (i.e., including but not limited to IgG4). In some embodiments, the heavy chain constant region has one or more of the following mutations: S228P; N297Q; and T299A (numbering according to Kabat). In some embodiments, the heavy chain constant region has one or more of the following mutations: L234A, L235A, and P329G (Kabat EU index numbering).

For example, WO 00/42072 describes antibodies with improved ADCC function in the presence of human effector cells, where the antibodies comprise amino acid substitutions in the Fc region thereof. Preferably, the antibody or antigen-binding fragment thereof with improved ADCC comprises substitutions at positions 298, 333, and/or 334 of the Fc region (Eu numbering of residues). Typically, the altered Fc region is a human IgG1 Fc region comprising or consisting of substitutions at one, two or three of these positions. Such substitutions are optionally combined with substitution(s) which increase Clq binding and/or CDC. Substitutions include an Asn297Ala mutation in IgG1 Fc.

Antibodies with altered Clq binding and/or complement dependent cytotoxicity (CDC) are described in WO 99/51642, U.S. Pat. Nos. 6,194,551, 6,242,195, and 6,538,124. The antibodies comprise an amino acid substitution at one or more of amino acid positions 270, 322, 326, 327, 329, 313, 333 and/or 334 of the Fc region thereof (Eu numbering of residues).

Antibodies with improved binding to the neonatal Fc receptor (FcRn), and increased half-lives, are described in WO 00/42072 and U.S. Patent Pub. No. 2005/0014934. These antibodies comprise an Fc region with one or more substitutions therein which improve binding of the Fc region to CEACAM1. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising an antibody variant with improved CEACAM1 binding comprises amino acid substitutions at one, two or three of positions 307, 380 and 434 of the Fc region thereof (Eu numbering of residues). In one embodiment, the antibody or antigen-binding fragment thereof has 307/434 mutations. Engineered antibodies that bind to CEACAM1 with three or more (e.g., four) functional antigen binding sites are also contemplated. See, e.g., U.S. Patent Pub. No. US 2002/0004587.

### Antibody Fragments and Types

In some embodiments of the aspects described herein, the CEACAM1 antibody fragment is a Fab fragment, which comprises or consist essentially a variable (V_{L}) and constant (C_{L}) domain of the light chain and a variable domain (V_{H}) and the first constant domain (C_{H}1) of the heavy chain.

In some embodiments of the aspects described herein, the CEACAM1 antibody fragment is a Fab′ fragment, which refers to a Fab fragment having one or more cysteine residues at the C-terminus of the CHI domain.

In some embodiments of the aspects described herein, the CEACAM1 antibody fragment is an Fd fragment comprising or consisting essentially of V_{H} and C_{H}1 domains.

In some embodiments of the aspects described herein, the CEACAM1 antibody portion is an Fd′ fragment comprising V_{H} and C_{H}1 domains and one or more cysteine residues at the C-terminus of the C_{H}1 domain.

Single-chain Fv or scFv antibody fragments comprise or consist essentially of the V_{H} and V_{L} domains of antibody, such that these domains are present in a single polypeptide chain. Generally, an Fv polypeptide further comprises a polypeptide linker between the V_{H} and V_{L} domains, which allows the scFv to form the desired structure for antigen binding. See, for example, Pluckthun, 113 Pharmacology Monoclonal Antibodies 269 (Rosenburg & Moore, eds., Springer-Verlag, New York, 1994). Accordingly, in some embodiments of the aspects described herein, the CEACAM1 antibody fragment is a Fv fragment comprising or consisting essentially of the V_{L} and V_{H} domains of a single arm of an antibody.

In some embodiments of the aspects described herein, the CEACAM1 antibody portion is a diabody comprising two antigen binding sites, comprising a heavy chain variable domain (V_{H}) connected to a light chain variable domain (V_{L}) in the same polypeptide chain.

In some embodiments of the aspects described herein, the CEACAM1 antibody portion is a dAb fragment comprising or consisting essentially of a V_{H} domain.

In some embodiments of the aspects described herein, the CEACAM1 antibody portion is a F(ab′)2 fragment, which comprises a bivalent fragment comprising two Fab′ fragments linked by a disulfide bridge at the hinge region.

Linear antibodies refer to the antibodies as described in Zapata et al., Protein Engin., 8(10):1057-1062 (1995). Briefly, these antibodies comprise a pair of tandem Fd segments (V_{H}-C_{H}1-V_{H}-C_{H}1), which, together with complementary light chain polypeptides, form a pair of antigen binding regions. Linear antibodies can be bispecific or monospecific. In some embodiments of the aspects described herein, the CEACAM1 antibody fragment is a linear antibody comprising a pair of tandem Fd segments (V_{H}-C_{H}1-V_{H}-C_{H}1) which, together with complementary light chain polypeptides, form a pair of antigen binding regions.

Various techniques have been developed and are available for the production of antibody fragments. Traditionally, these fragments were derived via proteolytic digestion of intact antibodies. See, e.g., Morimoto et al., 24 J. Biochem. Biophys. Meths. 107 (1992); Brennan et al., 229 Science 81 (1985). However, these fragments can now be produced directly by recombinant host cells. For example, antibody fragments can be isolated from the antibody phage libraries discussed herein. Alternatively, Fab′-SH fragments can be directly recovered from E. coli and chemically coupled to form F(ab′)2 fragments (Carter et al., 1992). According to another approach, F(ab′)2 fragments can be isolated directly from recombinant host cell culture. Other techniques for the production of antibody fragments will be apparent to the skilled practitioner. In other embodiments, the antibody fragment of choice is a single chain Fv fragment (scFv). See, for example, WO 93/16185.

In one embodiment, the antibody is a bispecific antibody comprising a complementary region that binds to CEACAM1 and a complementary region that binds to PD-1. In one embodiment, the antibody is a bispecific antibody comprising a complementary region that binds to CEACAM1 and a complementary region that binds to PD-L1. In one embodiment, the antibody is a bispecific antibody comprising a complementary region that binds to CEACAM1 and a complementary region that binds to TIM-3. In one embodiment, the antibody is a bispecific antibody comprising a complementary region that binds to CEACAM1 and a complementary region that binds to epidermal growth factor receptor (EGFR). In one embodiment, the antibody is a bispecific antibody comprising a complementary region that binds to CEACAM1 and a complementary region that binds to Fc gamma receptor.

Contemplated antibodies or antigen-binding fragments may have all types of constant regions, including IgM, IgG, IgD, and IgE, and any isotype, including IgG1, IgG2, IgG3, and IgG4. In one embodiment, the human isotype IgG1 is used. In another embodiment, the human isotype IgG4 is used. Light chain constant regions can be λ, or κ. The antibody or antigen-binding fragment thereof may comprise sequences from more than one class or isotype.

Also disclosed herein are chimeric antigen receptor T-cells (CAR T-cells) that bind to CEACAM1. In one embodiment, one or more of the CDRs of an anti-CEACAM antibody disclosed herein are grafted onto a chimeric antigen receptor (CAR) on a T-cell. Such a genetically modified T-cell utilizes the CAR, also known as a chimeric T cell receptor, to target antigens expressed on tumor cells in a human leukocyte antigen-independent manner.

### Antibody Binding

The human CEACAM1 gene produces 11 isoforms by alternative splicing. Each isoform has one variable (V)-like Ig domain at the amino (N) end of the protein. With the exception of CEACAM1-1L and CEACAM1-1S isoforms, the various isoforms also have 2 or 3 constant C2-like Ig domains. Eight CEACAM1 isoforms are anchored to the cellular membrane via a transmembrane domain and three CEACAM1 isoforms (CEACAM1-4C1, -3 and -3C2) lack the transmembrane domain and are secreted. Two isoforms (CEACAM1-3AL and -3AS) have an Alu family repeat sequence (A) between the constant C2-like Ig domains and the transmembrane domain. The transmembrane CEACAM1 isoforms also possess a long (L) or short (S) cytoplasmic domain determined by inclusion or exclusion of CEACAM1 exon 7 in the message. The CEACAM1 L cytoplasmic domain has two ITIM motifs, which are unique to CEACAM1 among the CEACAM family members. In one aspect, provided are CEACAM1 antibodies or antigen-binding fragments thereof, including the antibodies described herein by their structural characteristics, that bind to the extracellular, variable (V)-like Ig domain at the amino (N) end of the protein (N-domain) of CEACAM1, a domain that is common to all isoforms of CEACAM1, including CEACAM1 isoforms 1L, 1S, 3L, 3S, 4L, 4S, 3A1, 3AS, 3, 4C1, and 4C2. In some embodiments, the provided antibodies and antigen-binding fragments thereof bind to human CEACAM1. In some embodiments, the provided antibodies and antigen-binding fragments thereof bind to mammalian CEACAM1. The sequence of the full-length form of CEACAM1 (NCBI Reference Sequence NP 001703.2; UNIPROT ID P13688) is provided as SEQ ID NO:23 (signal sequence: residues 1-34 of SEQ ID NO:23; Ig-V N domain: residues 35-142 of SEQ ID NO:23. The mature form of CEACAM1 (without signal sequence) is provided as SEQ ID NO:24.

As used herein, “binding” of an antibody or antigen binding fragment thereof to CEACAM1, an epitope on CEACAM1, or, in certain embodiments described below, particular residues on CEACAM1, includes the selective interaction of the antibody or antigen binding fragment thereof with CEACAM1. Binding therefore includes, e.g., primary and secondary interactions including hydrogen bonds, ionic interactions, salt bridges, as well as hydrophilic and hydrophobic interactions.

In certain embodiments, the CEACAM1 antibodies or antigen-binding fragments thereof described herein bind to CEACAM1 with a K_{D} of 10⁻⁵ to 10⁻¹² mol/l, 10⁻⁶ to 10¹² mol/l, 10⁻⁶ to 10⁻⁹ mol/l, 10⁻⁷ to 10⁻¹² mol/l, 10⁻¹² to 10⁻¹² mol/l, 10⁻⁹ to 10⁻¹² mol/l, 10⁻¹⁰ to 10⁻¹ mol/l, or 10⁻¹¹ to 10⁻¹² mol/l. In other embodiments, the CEACAM1 antibodies or antigen-binding fragments thereof described herein bind to CEACAM1 with a K_{D} of 10⁻⁵ to 10⁻¹¹ mol/l, 10⁻⁶ to 10⁻¹¹ mol/l, 10⁻⁷ to 10⁻¹¹ mol/l, 10⁻⁸ to 10⁻¹¹ mol/l, 10⁻¹¹ to 10⁻¹¹ mol/l, or 10⁻¹⁰ to 10⁻¹¹ mol/l. In other embodiments, the CEACAM1 antibodies or antigen-binding fragments thereof described herein bind to CEACAM1 with a K_{D} of 10⁵ to 10⁻¹⁰ mol/l, 10⁻⁶ to 10⁻¹⁰ mol/l, 10⁻⁷ to 10⁻¹⁰ mol/l, 10⁻⁸ to 10⁻¹⁰ mol/l, or 10⁻⁹ to 10⁻¹⁰ mol/l. In other embodiments, the CEACAM1 antibodies or antigen-binding fragments thereof described herein bind to CEACAM1 with a K_{D} of 10⁻⁵ to 10⁻⁸ mol/l, 10⁻⁶ to 10⁻⁸ mol/l, or 10⁻⁷ to 10⁻⁹ mol/l.

The term “specificity” herein refers to the ability of an antibody or antigen-binding fragment thereof, such as an anti-CEACAM1 antibody or antigen-binding fragment thereof, to recognize an epitope within CEACAM1, while only having little or no detectable reactivity with other portions of CEACAM1. Specificity can be relatively determined by competition assays or by epitope identification/characterization techniques described herein or their equivalents known in the art.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a particular spatial conformation. An “epitope” includes the unit of structure conventionally bound by an immunoglobulin V_{H}/V_{L} pair. Epitopes define the minimum binding site for an antibody or antigen-binding fragment thereof, and thus represent the target of specificity of an antibody or antigen-binding fragment thereof. In the case of a single domain antibody, an epitope represents the unit of structure bound by a variable domain in isolation.

In a particular embodiment, the contemplated antibody or antigen-binding fragment specifically binds to the same epitope as antibody He14/L1.

In one aspect, the invention provides antibodies and antigen-binding fragments thereof, including the antibodies described herein by their structural characteristics, wherein the antibodies and antigen-binding fragments thereof specifically bind to at least part of the homophilic binding domain on CEACAM1 (i.e., in the portion of the CEACAM1 protein that is involved in formation of a CEACAM1:CEACAM1 homodimer), thereby blocking CEACAM1 homophilic interactions. CEACAM1 residues involved in homodimerization are shown in Table 1.

TABLE 1
Interactions observed in the crystal structure of the human
CEACAM1 wildtype dimer (PDB ID 4QXW). The side chains of
residues S32, Y34, Q44, Q89, N97, and E99 form a hydrogen
bonding network at the GFCC' interface that includes
additional side-chain to main-chain backbone interactions
between S32 to L95, Q44 to N97, and E99 to G41 and hydrophobic
interactions by residues F29, V39, and I91.

| Interaction | hCEACAM1 (A) | hCEACAM1 (B) |
|---|---|---|
| number | residues | residues |
| 1 | A: L95 | B: S32 |
| 2 | A: E99 | B: G41 |
| 3 | A: L95 | B: Q44 |
| 4 | A: Y34 | B: Q89 |
| 5 | A: Q89 | B: Q89 |
| 6 | A: Q44 | B: N97 |
| 7 | A: S32 | B: N97 |
| 8 | A: Y34 | B: N97 |
| 9 | A: Q89 | B: N97 |
| 10 | A: S32 | B: L95 |
| 11 | A: G41 | B: E99 |
| 12 | A: Q44 | B: L95 |
| 13 | A: Q89 | B: Q89 |
| 14 | A: Q89 | B: Y34 |
| 15 | A: N97 | B: Q44 |
| 16 | A: N97 | B: S32 |
| 17 | A: N97 | B: Y34 |

In some embodiments, the CEACAM antibody or antigen-binding fragment thereof binds to a residue in CEACAM1's GFCC' face such as F29, Y31, Q44, Y48, T56, Q89, D94, L95, and/or N97 of SEQ ID NO:24.

In some embodiments, the CEACAM antibody or antigen-binding fragment thereof binds to a residue in CEACAM1's ABED face such M7, E16, Q27, Y48, Q53, Y68, or N70 of SEQ ID NO:24.

As used herein, a “blocking” antibody or an antibody “antagonist” is one that inhibits or reduces biological activity of the antigen to which it binds. For example, in some embodiments, a CEACAM1 antagonist antibody or antigen-binding fragment thereof binds CEACAM1 and inhibits activity of CEACAM1 and/or binding of CEACAM1 to heterologous binding partners such as other CEACAM proteins or TIM-3. Inhibition of activity and inhibition of binding includes partial inhibition. Methods for the identification of CEACAM1 antibodies that block CEACAM1 homophilic and heterophilic interactions are described herein and are known to the ones skilled in the art. For instance, competing. cross-blocking, and cross-blocked antibodies can be identified using any suitable method known in the art, including competition ELISAs or BIACORE® assays where binding of the competing or cross-blocking antibody to human CEACAM1 prevents the binding of an antibody disclosed herein or vice versa.

In certain embodiments, not all CDRs are directly involved in binding to the antigen. In one embodiment, four out of six CDRs of the CEACAM1 antibody or antigen-binding fragment thereof make contact with the antigen. In one embodiment, five out of six CDRs of the CEACAM1 antibody or antigen-binding fragment thereof make contact with the antigen. In one embodiment, six out of six CDRs of the CEACAM1 antibody or antigen-binding fragment thereof make contact with the antigen.

CEACAM family members are expressed widely expressed on a variety of cell types (especially leukocytes), affecting a magnitude of cellular functions. For instance, CEACAM1 is expressed on epithelial cells, endothelial cells, lymphocytes, and myeloid cells, CEACAM3 is expressed on granulocytes and neutrophils, CEACAM5 expressed on epithelial cells, and CEACAM6 is expressed on epithelial cells and granulocytes. However, the N-domain of CEACAM1 is about 90% similar to the N-domains of CEACAM family members 3, 5, and 6, making it difficult to target CEACAM1 selectively.

Despite the high similarity of N-domains among CEACAM family members, in some embodiments, antibodies or antigen-binding fragments thereof provided herein, including the antibodies described herein by their structural characteristics, are selective for CEACAM1. By selectively targeting CEACAM1, embodiments of the invention may avoid undesired interfering e.g., with the broad activating function of CEACAM3.

The terms “selective” and “selectivity” herein refer to the preferential binding of an antibody or antigen-binding fragment thereof (i.e., a CEACAM1 antibody or antigen-binding fragment thereof), for a particular region, target, or peptide; typically, a region or epitope in CEACAM1, as opposed to one or more other biological molecules, including other CEACAM family members.

In some embodiments, the contemplated CEACAM1 antibody or antigen-binding fragment thereof does not exhibit significant binding to CEACAM3, CEACAM5, CEACAM6 and/or CEACAM8. In some embodiments, the contemplated CEACAM1 antibody or antigen-binding fragment thereof does not exhibit detectable binding to CEACAM3, CEACAM5, CEACAM6 and/or CEACAM8. In some embodiments, the contemplated CEACAM1 antibody or antigen-binding fragment thereof binds CEACAM1 with an affinity that is at least 10 times, such as at least 100 times, and at least 1000 times, and up to 10,000 times or more stronger than the affinity with which the contemplated CEACAM1 antibody or antigen-binding fragment thereof binds to another target or polypeptide.

As used herein, “affinity”, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof. The smaller the value of the K_{D}, the stronger the binding strength between an antigenic determinant and the antigen-binding molecule. Alternatively, the affinity can also be expressed as the affinity constant (K_{D}), which is 1/K_{D}). As will be clear to the skilled person, affinity can be determined in a manner known per se, depending on the specific antigen of interest.

In one aspect, the invention provides antibodies and antigen-binding fragments thereof, including the antibodies described herein by their structural characteristics, wherein the antibodies and antigen-binding fragments thereof specifically bind to at least part of the binding site on CEACAM1 for one or more other members of the CEACAM family, thereby blocking CEACAM1 interactions with the one or more other members of the CEACAM family. These CEACAM family members include, but are not limited to, CEACAM5 and CEACAM8 (Ramani et al, Anal. Biochem. Jan. 15, 2012; 420(2); 127-38; Scheffrahn et al, J. Immunol. May 15, 2002; 168(10); 5139-46).

In one aspect, the invention provides antibodies and antigen-binding fragments thereof, including the antibodies described herein by their structural characteristics, wherein the antibodies and antigen-binding fragments thereof specifically bind to at least part of the binding site on CEACAM1 for a member of the TIM family, thereby blocking CEACAM1 interaction with the TIM family member. In some embodiments, this TIM family member is TIM-1, TIM-3, or TIM-4. In some embodiments, the CEACAM1 antibody or antigen-binding fragment thereof specifically binds to one or more of CEACAM1 residues Y34, G41, N42, Q44, Q89, S93, D94, V96, and/or N97 of SEQ ID NO:17, residues which have been indicated to be involved in CEACAM1 binding to TIM-3 (Huang et al., Nature. 2015 Jan. 15; 517(7534):386-90).

In one aspect, the invention provides antibodies and antigen-binding fragments thereof, including the antibodies described herein by their structural characteristics, wherein the antibodies and antigen-binding fragments thereof specifically bind to at least part of the binding site on CEACAM1 for a pathogen, thereby blocking the interaction between CEACAM1 and the pathogen.

In one aspect, the invention provides antibodies and antigen-binding fragments thereof, including the antibodies described herein by their structural characteristics, wherein the antibodies and antigen-binding fragments thereof specifically bind to at least part of the binding site on CEACAM1 for a bacterial adhesive surface protein (adhesin), thereby blocking the interaction between CEACAM1 and the adhesin. In certain embodiments, the adhesin is expressed on the surface of a CEACAM1-binding pathogenic bacterium including, but not limited to, Escherichia coli, particularly Diffusively Adhering Escherichia coli (DAEC), Neisseria gonorrhoeae, N. meningitidis, commensal Neisseria, Moraxella catarrhalis, Haemophilus influenza, Haemophilus aegyptius, Helicobacter pylori, Fusobacterium sp., Salmonella sp, and/or Streptococcus agalactiae. In one embodiment, the adhesin is the β protein from S. agalactiae.

In one embodiment, the CEACAM1 antibody or antigen-binding fragment thereof disrupts the interaction between CEACAM1 and Streptococcus agalactiae (Group B Streptococcus). In one embodiment, the CEACAM1 antibody or antigen-binding fragment thereof disrupts the interaction between CEACAM1 and the β protein from S. agalactiae. Interactions observed in the crystal structure of the BIgI domain of the β protein from S. agalactiae and human CEACAM1 are shown in Table 2. In one embodiment, the CEACAM1 antibody or antigen-binding fragment thereof disrupts one of more of the interactions shown in Table 2.

TABLE 2
Interactions observed in the crystal structure
of the BIgI domain of the β protein from
S. agalactiae and human CEACAM1 (PDB ID 6V3P).

| Interaction | Adhesin (C) Residues | hCEACAM1 (B) Residues |
|---|---|---|
| 1 | C: PHE 41 | B: SER 93 |
| 2 | C: ASP 54 | B: TYR 34 |
| 3 | C: THR 58 | B: LEU 95 |
| 4 | C: TYR 60 | B: ASP 94 |
| 5 | C: VAL 52 | B: GLN 44 |
| 6 | C: ASP 54 | B: GLY 41 |

In one embodiment, the CEACAM1 antibody or antigen-binding fragment thereof disrupts the interaction between CEACAM1 and HopQ expressed on the surface of Helicobacter pylori. In one embodiment, the CEACAM1 antibody or antigen-binding fragment specifically binds to one or more of CEACAM1 residues F29, Y34, N42, Q89, and N97, which have been predicted to be involved in CEACAM1 binding to HopQ. Interactions observed in the crystal structure of HopQ and hCEACAM1 are shown in Table 3. In one embodiment, the CEACAM1 antibody or antigen-binding fragment thereof disrupts one of more of the interactions shown in Table 3.

TABLE 3
Interactions observed in the crystal structure
of HopQ and hCEACAM1 (PDB ID 6AW2).

| Interactions | HopQ (B) Residues | hCEACAM1 (A) Residues |
|---|---|---|
| 1 | B: SER 135 | A: LEU 95 |
| 2 | B: THR 136 | A: LEU 95 |
| 3 | B: THR 136 | A: LEU 95 |
| 4 | B: ASN 137 | A: ASN 97 |
| 5 | B: SER 138 | A: ASN 97 |
| 6 | B: SER 138 | A: ASN 97 |
| 7 | B: ASN 145 | A: VAL 39 |
| 8 | B: THR 149 | A: GLN 89 |
| 9 | B: ALA 243 | A: SER 93 |
| 10 | B: THR 370 | A: ASP 94 |
| 11 | B: THR 149 | A: GLN 44 |
| 12 | B: ASN 145 | A: GLN 89 |
| 13 | B: SER 135 | A: ASN 97 |
| 14 | B: THR 136 | A: ASN 97 |
| 15 | B: SER 135 | A: ASN 97 |

In another embodiment, the CEACAM1 antibody or antigen-binding fragment thereof disrupts the interaction between CEACAM1 and an opacity-associated (Opa) adhesin protein expressed on the surface of Neisseria sp, including, but not limited to, Opa₅₂, Opa₆₅, Opa₆₈, Opa₇₀, Opa₇₂, Opa₇₃, Opa₇₄, and Opa₇₅. In one embodiment, the CEACAM1 antibody or antigen-binding fragment specifically binds to one or more of CEACAM1 residues Q44 and A49, which have been predicted to be involved in CEACAM1 binding to neisserial Opa proteins.

In another embodiment, the CEACAM1 antibody or antigen-binding fragment thereof disrupts the interaction between CEACAM1 and Opa-like protein OlpA expressed on the surface of Moraxella sp.

In one embodiment, the CEACAM1 antibody or antigen-binding fragment thereof disrupts the interaction between CEACAM1 and Haemophilus influenza OMP P1. In one embodiment, the CEACAM1 antibody or antigen-binding fragment specifically binds to one or more of CEACAM1 residues Q44 and A49, which have been predicted to be involved in CEACAM1 binding to Haemophilus influenza OMP P1.

In another embodiment, the CEACAM1 antibody or antigen-binding fragment thereof disrupts the interaction between CEACAM1 and Haemophilus aegyptius OMP P1. In one embodiment, the CEACAM1 antibody or antigen-binding fragment specifically binds to CEACAM1 residue F29, which has been predicted to be involved in CEACAM1 binding to Haemophilus aegyptius OMP P1.

In another embodiment, the CEACAM1 antibody or antigen-binding fragment thereof disrupts the interaction between CEACAM1 and C. albicans.

In another embodiment, the invention provides methods of using the CEACAM1 antibodies or antigen-binding fragments thereof described herein for inhibiting binding of CEACAM1 to a filial nematode, the method comprising contacting CEACAM1 with a CEACAM1 antibody or antigen-binding fragment thereof described herein. In one embodiment, the filial nematode is Wucheria bancrofti.

### Antibody Conjugates

In some embodiments of the aspects described herein, the antibody or antigen-binding fragment thereof that bind to CEACAM1 are conjugated to a functional moiety. Examples of useful functional moieties include, but are not limited to, a blocking moiety, a detectable moiety, a diagnostic moiety, a targeting, and a therapeutic moiety.

Exemplary blocking moieties include moieties of sufficient steric bulk and/or charge such that reduced glycosylation occurs, for example, by blocking the ability of a glycosidase to glycosylate the antibody or antigen-binding fragment thereof. The blocking moiety may additionally or alternatively, reduce effector function, for example, by inhibiting the ability of the Fc region to bind a receptor or complement protein. Preferred blocking moieties include cysteine adducts and PEG moieties.

In a preferred embodiment, the blocking moiety is a cysteine, preferably a cysteine that has associated with a free cysteine, e.g., during or subsequent to the translation of the Fc containing polypeptide, e.g., in cell culture. Other blocking cysteine adducts include cystine, mixed disulfide adducts, or disulfide linkages.

In another preferred embodiment, the blocking moiety is a polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight, but these characteristics can affect the biological properties. Polymer average molecular weights particularly useful for decreasing clearance rates in pharmaceutical applications are in the range of 2,000 to 35,000 Daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically-active molecule.

Examples of detectable moieties which are useful in the methods and antibodies and antigen-binding fragments thereof contemplated by the invention include fluorescent moieties or labels, imaging agents, radioisotopic moieties, radiopaque moieties, and the like, e.g., detectable labels such as biotin, fluorophores, chromophores, spin resonance probes, or radiolabels. Exemplary fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties which are useful in the methods and antibodies and antigen-binding fragments thereof contemplated by the invention include detectable moieties suitable for revealing the presence of a disease or disorder. Typically, a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties which are useful in the methods and antibodies and antigen-binding fragments thereof contemplated by the invention include, for example, anti-inflammatory agents, anti-cancer agents, anti-neurodegenerative agents, or anti-infective agents. The functional moiety may also have one or more of the above-mentioned functions.

Exemplary therapeutic moieties include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high-energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary therapeutic moieties also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary therapeutic moieties also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary therapeutic moieties also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Exemplary therapeutic moieties also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g., paclitaxel, docetaxel, taxane), nocodazole, rhizoxin, dolastatins (e.g. Dolastatin-10, -11, or -15), colchicine and colchicinoids (e.g. ZD6126), combretastatins (e.g. Combretastatin A-4, AVE-6032), and vinca alkaloids (e.g. vinblastine, vincristine, vindesine, and vinorelbine (navelbine)); anti-angiogenesis compounds such as Angiostatin KI-3, DL-α-difluoromethyl-ornithine, endostatin, fumagillin, genistein, minocycline, staurosporine, and (±)-thalidomide.

Exemplary therapeutic moieties also include hormones and hormone antagonists, such as corticosteroids (e.g. prednisone), progestins (e.g. hydroxyprogesterone or medroprogesterone), estrogens, (e.g. diethylstilbestrol), antiestrogens (e.g. tamoxifen), androgens (e.g. testosterone), aromatase inhibitors (e.g. aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary therapeutic moieties also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary therapeutic moieties also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary therapeutic moieties also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other cytotoxins that are compatible with the teachings herein include auristatins (e.g., auristatin E and monomethylauristan E), calicheamicin, gramicidin D, maytansanoids (e.g. maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

To increase the half-life of the antibodies or polypeptide containing the amino acid sequences described herein, one can attach a salvage receptor binding epitope to the antibody or antigen-binding fragment thereof (especially an antibody fragment), as described, e.g., in U.S. Pat. No. 5,739,277. The term “salvage receptor binding epitope” may refer to an epitope of the Fc region of an IgG molecule (e.g., IgG1, IgG2, IgG3, or IgG4) that is responsible for increasing the in vivo serum half-life of the IgG molecule (e.g., Ghetie et al., 18 Ann. Rev. Immunol. 739 (2000). Antibodies with substitutions in an Fc region thereof and increased serum half-lives are also described in WO 00/42072, WO 02/060919; Shields et al., 276 J. Biol. Chem. 6591 (2001); Hinton, 279 J. Biol. Chem. 6213-6216 (2004). For example, a nucleic acid molecule encoding the salvage receptor binding epitope can be linked in frame to a nucleic acid encoding a polypeptide sequence described herein so that the fusion protein expressed by the engineered nucleic acid molecule comprises the salvage receptor binding epitope and a polypeptide sequence described herein. In another embodiment, the serum half-life can also be increased, for example, by attaching other polypeptide sequences. For example, antibodies or antigen-binding fragments thereof useful in the methods of the invention can be attached to serum albumin or a portion of serum albumin that binds to the CEACAM1 receptor or a serum albumin binding peptide so that serum albumin binds to the antibody or antigen-binding fragment thereof, e.g., such polypeptide sequences are disclosed in WO 01/45746. In one embodiment, the half-life of a Fab is increased by these methods. See also, Dennis et al., 277 J. Biol. Chem. 35035 (2002), for additional serum albumin binding peptide sequences.

Other types of functional moieties are known in the art and can be readily used in the methods and compositions of the present invention based on the teachings contained herein.

### Nucleic Acids

Also provided herein are nucleic acids encoding CEACAM1 antibodies and antigen-binding fragments thereof, as well as vectors, host cells, and expression systems. The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The nucleic acids encoding CEACAM1 antibodies and antigen-binding fragments thereof may be, e.g., DNA, cDNA, RNA, synthetically produced DNA or RNA, or a recombinantly produced chimeric nucleic acid molecule comprising any of those polynucleotides either alone or in combination. For example, provided is an expression vector comprising a polynucleotide sequence encoding a CEACAM1 antibody or antigen-binding fragment thereof described herein operably linked to expression control sequences suitable for expression in a eukaryotic and/or prokaryotic host cell.

The term “vector” refers to a nucleic acid molecule capable of transporting another nucleic acid to which it has been linked. A “vector” includes, but is not limited to, a viral vector, a plasmid, an RNA vector or a linear or circular DNA or RNA molecule which may consists of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus.

A variety of expression vectors have been developed for the efficient synthesis of antibodies and antigen-binding fragments thereof in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of the contemplated CEACAM1 antibodies or antigen-binding fragments thereof.

### Antibody Preparation and Expression Systems

The antibodies or antigen-binding fragments thereof of the invention are typically produced by recombinant expression. Nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

The expression of the antibodies and antigen-binding fragments contemplated by the invention can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human. primate, hamster, rabbit, rodent, cow, pig, sheep, horse, goat, dog or cat origin, but any other mammalian cell may be used.

E. col is one prokaryotic host particularly useful for cloning the polynucleotides (e.g., DNA sequences) of the present invention. Other microbial hosts suitable for use include bacilli, such as Bacillus subtillis, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a CEACAM1 antibody or antigen-binding fragment thereof of the present invention with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989).

Any of a series of yeast gene expression systems incorporating promoter and termination elements from the actively expressed genes coding for glycolytic enzymes produced in large quantities when yeast is grown in mediums rich in glucose can be utilized to obtain recombinant CEACAM1 antibodies or peptides of the present invention. Known glycolytic genes can also provide very efficient transcriptional control signals. For example, the promoter and terminator signals of the phosphoglycerate kinase gene can be utilized.

Production of CEACAM1 antibodies or antigen-binding fragments thereof in insects can be achieved. For example, by infecting the insect host with a baculovirus engineered to express a transmembrane polypeptide by methods known to those of skill. See Ausubel et al., 1987, 1993.

In addition to microorganisms, mammalian tissue culture may also be used to express and produce the antibodies or antigen-binding fragments thereof of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, nucleotide sequences encoding antibodies or antigen-binding fragments thereof can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Additionally, plants have emerged as a convenient, safe and economical alternative main-stream expression systems for recombinant antibody production, which are based on large scale culture of microbes or animal cells. Antibodies or antigen-binding fragments thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560 and 6,512,162; and WO 0129242. Several plant-derived antibodies have reached advanced stages of development, including clinical trials (see, e.g., Biolex, NC).

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The antibodies and antigen-binding fragments thereof of the invention can be expressed using a single vector or two vectors. When the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

### Methods for Modulating CEACAM1 Activity

In one aspect, the invention provides methods of using the antibodies and antigen-binding fragments thereof described herein for decreasing the interaction between CEACAM1 and another member of the CEACAM family, including, but not limited to, CEACAM1, CEACAM5, and CEACAM8. In some embodiments, the antibody or antigen-binding fragment thereof disrupts the homophilic interaction between CEACAM1 monomers.

In another aspect, the invention provides methods of using the antibodies and antigen-binding fragments thereof of the invention for decreasing the interaction between CEACAM1 and a member of the TIM family, including but not limited to TIM-1, TIM-3, and TIM-4. In some embodiments, the antibody or antigen-binding fragment thereof disrupts the heterophilic interaction between CEACAM1 and TIM-3. Disruption of the interaction between CEACAM1 and TIM-3 by using the antibodies and antigen-binding fragments thereof contemplated by the invention may reverse CEACAM1 inhibitory functions while maintain TIM-3 activating functions.

In another aspect, the invention provides methods of using the antibodies and antigen-binding fragments thereof of the invention for decreasing the interaction between CEACAM1 and PD-1.

The embodiments of the invention are useful for reducing immunosuppression, e.g., T cell tolerance. By “reducing” is meant the ability to cause an overall decrease of about 20% or greater, 30% or greater, 40% or greater, 45% or greater, 50% or greater, of 55% or greater, of 60% or greater, of 65% or greater, of 70% or greater, or 75%, 80%, 85%, 90%, 95%, or greater, as compared to a control that is not treated. Immunosuppression can be mediated by immune inhibitory receptors expressed on the surface of an immune cell, and their interactions with their ligands. For example, cytotoxic CD8 T cells can enter a state of “functional exhaustion,” or “unresponsiveness” whereby they express inhibitory receptors that prevent antigen-specific responses, such as proliferation and cytokine production. Accordingly, by inhibiting the activity and/or expression of such inhibitory receptors, an immune response to a cancer or tumor that is suppressed, inhibited, or unresponsive. can be enhanced or uninhibited. Such enhancements or reversal of inhibition of the immune response can lead to greater T cell activity, responsiveness, and/or ability or receptiveness with regards to activation.

Methods of measuring T cell activity are known in the art. By way of non-limiting example, T cell tolerance can be induced by contacting T cells with recall antigen, anti-CD3 in the absence of costimulation, and/or ionomycin. Levels of, e.g., IL-27. LDH-A, RAB10, and/or ZAP70 (both intracellular or secreted) can be monitored, for example, to determine the extent of T cell tolerogenesis (with levels of IL-2, interferon-γ and TNF correlating with increased T cell tolerance). The response of cells pre-treated with, e.g., ionomycin, to an antigen can also be measured in order to determine the extent of T cell tolerance in a cell or population of cells, e.g., by monitoring the level of secreted and/or intracellular IL-2 and/or TNF-α (see, e.g., Macian et al. Cell 2002 109:719-731). Other characteristics of T cells having undergone adaptive tolerance include increased levels of Fyn and ZAP-70/Syk, Cbl-b, GRAIL, Ikaros, CREM (cAMP response element modulator), B lymphocyte-induced maturation protein-1 (Blimp-1), PD-1, CD5, and SHP2; increased phosphorylation of ZAP-70/Syk, LAT, PLCyl/2, ERK, PKC-Θ/IKBA; increased activation of intracellular calcium levels; decreased histone acetylation or hypoacetylation and/or increased CpG methylation at the IL-2 locus. Thus, in some embodiments, one or more of any of these parameters can be assayed to determine whether the antibodies or antigen-binding fragments thereof disclosed herein that inhibit CEACAM1 decrease immune tolerance. Reduction of T cell tolerance can also be assessed by examination of tumor infiltrating lymphocytes or T lymphocytes within lymph nodes that drain from an established tumor. Such T cells exhibit features of “exhaustion” through expression of cell surface molecules such as PD-1, TIM-3 or LAG-3. for example, and decreased secretion of cytokines such as interferon-γ. Accordingly, evidence that T cell tolerance has been reduced in the presence of a CEACAM1 antibodies or antigen-binding fragments thereof includes, e.g., increased quantities of T cells with (a) antigen specificity for tumor associated antigens (e.g., as determined by major histocompatibility complex class I or class II tetramers which contain tumor associated peptides) and (b) the capability of secreting high levels of interferon-γ and cytolytic effector molecules such as granzyme-B, relative to that observed in the absence of the inhibitory agent.

The CEACAM1 antibodies and antigen-binding fragments thereof are further useful for enhancing T cell expansion, activation, and proliferation.

In one aspect, the invention provides methods of using the antibodies and antigen-binding fragments thereof of the invention for decreasing the interaction between CEACAM1 and bacterial adhesins. In some embodiments, the antibodies and antigen-binding fragments thereof of the invention are effective in reducing and/or preventing the colonization of mammalian epithelia. In some embodiments, the adhesins are expressed by Escherichia coli, particularly Diffusively Adhering Escherichia coli (DAEC), Neisseria gonorrhoeae, N. meningitidis, commensal Neisseria, Moraxella catarrhalis, Haemophilus influenza, Haemophilus aegyptius, Helicobacter pylori, Fusobacterium sp., Salmonella sp, and/or Streptococcus. agalactiae. In one embodiment, the CEACAM1 antibody or antigen-binding fragment thereof disrupts the interaction between CEACAM1 and HopQ expressed on the surface of Helicobacter pylori. In one embodiment, the CEACAM1 antibody or antigen-binding fragment thereof disrupts the interaction between CEACAM1 and opacity-associated (Opa) adhesin proteins expressed on the surface of Neisseria sp. In In one embodiment, the CEACAM1 antibody or antigen-binding fragment thereof disrupts the interaction between CEACAM1 and embodiment, the CEACAM1 antibody or antigen-binding fragment thereof disrupts the interaction between CEACAM1 and OMP adhesin proteins expressed on the surface of Haemophilus sp. In In one embodiment, the CEACAM1 antibody or antigen-binding fragment thereof disrupts the interaction between CEACAM1 and embodiment, the CEACAM1 antibody or antigen-binding fragment thereof disrupts the interaction between CEACAM1 and an adhesin expressed on the surface of Streptococcus agalactiae. In one embodiment, the CEACAM1 antibody or antigen-binding fragment thereof disrupts the interaction between CEACAM1 and Streptococcus agalactiae Ig13-like β protein adhesin.

In one embodiment, the CEACAM1 antibody or antigen-binding fragment thereof disrupts the interaction between CEACAM1 and C. albicans. In one embodiment, the invention provides methods of using the CEACAM1 antibodies or antigen-binding fragments thereof described herein for inhibiting binding of CEACAM1 to a filial nematode. In one embodiment, the filial nematode is Wucheria bancrofti.

### Methods of Treatment

In one aspect, the invention provides for CEACAM1 antibodies and antigen-binding fragments thereof that are also useful for the treatment of subjects in need thereof.

In the methods described herein, a therapeutically effective amount of an antibody or antigen-binding portions thereof set forth herein is administered to a mammal in need thereof. Although antibodies or antigen-binding portions thereof set forth herein are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody or antigen-binding portions thereof set forth herein that, when administered to a mammal, is effective in producing the desired therapeutic effect.

In some aspects, the antibody or antigen-binding fragment thereof binds to CEACAM1 expressed by an exhausted T cell or natural killer (NK) cells, thereby recovering T cell and NK cell activity and leading to increased anti-tumor responses. In other aspects, the antibody or antigen-binding fragment thereof binds to CEACAM1 expressed by a tumor cell, thereby inhibiting tumor cell metastasis and the formation of a cancer stem cell niche. In yet another aspects, the antibody or antigen-binding fragment thereof binds to CEACAM1 expressed by macrophage associated with fibrosis in the tumor environment thereby inhibiting fibrosis. In another aspects, the antibody or antigen-binding fragment thereof binds to CEACAM1 expressed by other stromal cells in the tumor microenvironment such as vascular endothelium cells, thereby inhibiting angiogenesis.

As such, also provided herein are methods of treating a subject having a cancer or tumor and/or reducing tumor growth, comprising administering an effective amount of a CEACAM1-antibody or antigen-binding fragment thereof provided herein. “Reducing” includes inhibiting and/or reversing and can refer to, for example, the symptoms of the disorder being treated, the presence or size of metastases or micrometastases, the size of the primary tumor, the presence or the size of the dormant tumor.

The term “cancer” refers to or describes the physiological condition in mammals that is typically characterized by unregulated cell growth. Included in this definition are benign and malignant cancers, as well as dormant tumors or micrometastases. Accordingly, the term “cancer” as used herein refers to an uncontrolled growth of cells, which interferes with the normal functioning of the bodily organs and systems, including cancer stem cells and tumor vascular niches. A subject that has a cancer is a subject having objectively measurable cancer cells present in the subject's body. Included in this definition are benign and malignant cancers, as well as dormant tumors or micrometastases. Cancers that migrate from their original location and seed vital organs can eventually lead to the death of the subject through the functional deterioration of the affected organs. Hematopoietic cancers, such as leukemia, are able to out-compete the normal hematopoietic compartments in a subject, thereby leading to hematopoietic failure (in the form of anemia, thrombocytopenia and neutropenia) ultimately causing death.

By “subject” is meant a mammal, including, but not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

The embodiments of the invention may be used for treating metastasis, which relates to the spreading of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Also contemplated are methods of reducing cancer stemness comprising the administration of the CEACAM1 antibodies or antigen-binding fragments thereof disclosed herein. Cancer stemness may refer to the ability of a cell to self-renew and to generate an additional, phenotypically distinct cell type. Cancer stem cells (CSCs) are cancer cells that exhibit stem-cell like properties. CSCs often exhibit at least one hallmark of cancer, and is capable of generating at least one additional, phenotypically distinct cell type. Furthermore, cancer stem cells are capable of both asymmetric and symmetric replication. It is appreciated that a cancer stem cell may result from differentiated cancer cells that acquire stemness traits and/or stem cells that acquire phenotypes associated with cancer cells. Alternatively, cancer stem cells can reconstitute non-stromal cell types within a tumor.

CEACAM1 is expressed by many tumor types and CEACAM1 may regulate the growth and metastatic behavior of the tumor. In another embodiment, CEACAM1 inhibition will decrease tumor growth and metastasis.

CEACAM1 expression on subsets of macrophages is associated with fibrosis during carcinogenesis. In a further embodiment, CEACAM1 inhibition will decrease tumor-associated fibrosis.

Cancers that may be treated by the compositions and methods contemplated by the invention include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated include, but are not limited to benign and malignant tumors, and malignancies e.g., sarcomas, carcinomas, and melanomas. Adult tumors/cancers and pediatric tumors/cancers are also included. Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; vulvar sarcomas; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. A patient can have more than one type of cancer.

The efficacy of the treatment methods for cancer comprising therapeutic formulations of the compositions comprising the antibodies and antigen-binding fragments thereof described herein can be measured by various endpoints commonly used in evaluating cancer treatments, including but not limited to, tumor regression, tumor weight or size shrinkage, time to progression, duration of survival, progression free survival, overall response rate, duration of response, and quality of life. In the case of cancers, the therapeutically effective amount of the recombinant CEACAM1-antibody or antigen-binding fragment thereof can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. In cases where a patient has more than one type of cancer, the therapeutically effective amount of the recombinant CEACAM1-antibody or antigen-binding fragment thereof is an amount effective in treating at least one of the cancers. To the extent the recombinant CEACAM1-antibody or antigen binding-fragment thereof acts to prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

Checkpoint proteins interact with specific ligands that send a signal into the T cell and switch off or inhibit T cell function. By expressing high levels of checkpoint proteins on their surface, cancer cells can control the function of T cells that enter the tumor microenvironment, thus suppressing the anticancer immune response. The immune checkpoint protein Programmed Death-1 (PD-1) is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1 (Freeman et al., 2000; Latchman et al., 2001). Inhibition of the PD-1/PD-L1 interaction can promote potent antitumor activity. Examples of PD-1 inhibitors include, but are not limited to, Pembrolizumab (MK-3475), Nivolumab (MDX-1106), Cemiplimab-rwlc (REGN2810), Pidilizumab (CT-011), Spartalizumab (PDR001), tislelizumab (BGB-A317), PF-06801591, AK105, BCD-100, BI 754091, JS001, LZM009, MEDI0680, MGA012, Sym021, TSR-042. Examples of PD-L1 inhibitors include, but are not limited to, Atezolizumab (MPDL3280A), Durvalumab (MEDI4736), Avelumab (MSB0010718C), BGB-A333, CK-301, CS1001, FAZ053, KN035, MDX-1105, MSB2311, SHR-1316.

However, there is a significant population of cancer patients receiving checkpoint inhibitor therapy that (1) fail to respond to this type of therapy (innate or primary resistance) or that (2) initially respond but eventually develop disease progression (secondary or acquired resistance). Resistant cancer may also be referred to as refractory cancer. As shown in the Examples below, tumor associated cells isolated from patients with acquired resistance to PD-1/PD-L1 inhibitors upregulate CEACAM1 expression relative tumor associated cells isolated from naïve patients, that had not been exposed to PD-1 inhibitors. When CEACAM1 is expressed in the setting of acquired resistance, the CEACAM1 bearing cells are more like likely to be effector memory rather than central memory cells, consistent with a reduction of an anti-cancer response in the resistant patients.

As such, also provided herein are methods of using CEACAM1 antibodies and antigen-binding fragments thereof, including, but not limited to the specific CEACAM1 antibodies and antigen-binding fragments thereof provided herein, for the treatment of patients with resistance to checkpoint inhibitors such as inhibitors of PD-1, PD-L1, and/or CTLA-4. In some embodiments, the CEACAM1 antibody used in the treatment of patients with resistance to inhibitors of PD-1, PD-L1, and/or CTLA-4 is He14/L1. In some embodiments, the resistance is innate or primary resistance. In some embodiments, the resistance is secondary or acquired resistance. In some embodiments, the administered CEACAM1 antibodies, including, but not limited to the CEACAM1 antibodies and antigen-binding fragments thereof provided herein, reverse T cell exhaustion in patients resistant to checkpoint inhibitor therapy. Any cancer exhibiting PD-1, PDL-1 and/or CTLA-4 resistance is suitable for treatment with the methods of the invention. In some embodiments, the CEACAM1 antibody or antigen-binding fragment is administered to a patient that has not previously receive checkpoint inhibitor therapy.

In another aspect, the invention provides for the use of the CEACAM1 antibodies and antigen-binding fragments provided herein in the treatment of patients with resistance to therapy with other checkpoint inhibitors, including but not limited to, inhibitors of PD-L2, B7-H3, B7-H4, BTLA, HVEM, GAL9, LAG3, TIM-3, VISTA, KIR, 2B4 (belongs to the CD2 family of molecules and is expressed on all NK, γδ, and memory CD8⁺ (αβ) T cells), CD160 (also referred to as BY55), CGEN-15049, CHK1 and CHK2 kinases, A2aR and various B-7 family ligands (including, but are not limited to, B7-1, B7-2, B7-DC, B7-H1, B7-H2, B7-H3, B7-H4, B7-H5, B7-H6 and B7-H7).

In another aspect, the invention provides methods of using the CEACAM1 antibodies and antigen-binding fragments thereof disclosed herein for the treatment of a subject in need of reducing and/or preventing the colonization of mammalian epithelia with Candida albicans and/or bacteria expressing bacterial adhesins (including, but not limited to, Escherichia coli, particularly Diffusively Adhering Escherichia coli (DAEC), Neisseria gonorrhoeae. N. meningitidis, commensal Neisseria, Moraxella catarrhalis, Haemophilus influenza, Haemophilus aegyptius, Helicobacter pylori, Fusobacterium sp., Salmonella sp., and/or Streptococcus. agalactiae.

In another aspect, the invention provides methods of using the CEACAM1 antibodies and antigen-binding fragments thereof disclosed herein for the treatment of a subject in need of reducing and/or preventing the infection with a filial nematode such as Wucheria bancrofti. In another aspect, the invention provides methods of using the CEACAM1 antibodies and antigen-binding fragments thereof disclosed herein for the treatment of a subject in need of reducing and/or preventing the development of lymphedema and/or hydrocele associated with an infection with a filial nematode such as Wucheria bancrofi. In one embodiment, the invention provides methods of using the CEACAM1 antibodies or antigen-binding fragments thereof described herein for reducing invasion of a subject's lymphatic system with a filarial worm in a subject in need thereof. In one embodiment, the filial nematode is Wucheria bancrofti. A subject may be infected with more than one of a bacterium expressing a bacterial adhesin, Candida albicans, an influenza virus and/or a filial nematode.

In another embodiment, the invention provides methods of using the CEACAM1 antibodies or antigen-binding fragments thereof described herein for reducing the invasion of a subject's lymphatic system with cancer cells in a subject in need thereof.

### Screening Methods

Provided herein are also methods of identifying patent populations who are likely to respond to treatment with the CEACAM1 antibodies and antibody-fragments provided herein, including but not limited to, He14/L1.

In some embodiments, a cancer patient is screened for CEACAM1 expression on certain cell types, including T cells, NK cells, B cells, tumor cells, fibroblastic cells or other cells in the tumor microenvironment such as macrophages. In some embodiments, cancer patients that show an increased expression of CEACAM1 on certain cell types as compared to a control are selected for treatment with the CEACAM1 antibodies and antibody-fragments provided herein. A “control” level of CEACAM1 expression can refer to the level of CEACAM1 expression in one or more individuals to do not have cancer. The level may be measured on an individual-by-individual basis, or on an aggregate basis such as an average. In some embodiments, the control level of CEACAM1 expression from the same individual whose condition is being monitored but is obtained at a different time. In certain embodiments, a “control” level can refer to a level obtained from the same patient at an earlier time, e.g., weeks, months, or years earlier. In some embodiment, the control level is obtained from a patient before the patient received any cancer therapy. In some embodiment, the control level is obtained from a patient before the patient received treatment with a checkpoint inhibitor.

In some embodiments, CEACAM1 expression is determined for patients resistant to checkpoint inhibitor therapy, including, but not limited to therapy with PD-1/PD-L1/CTLA-4 inhibitors. In some embodiments, patients that are resistant to checkpoint inhibitor therapy and that show an increased expression of CEACAM1 on certain cell types as compared to a control are selected for treatment with the CEACAM1 antibodies and antibody-fragments provided herein, including but not limited to, He14/L1.

In some embodiments, a patient is assayed for an allelic variant of human CEACAM1. Based on which allelic variant of human CEACAM1 the patient expresses, more or less anti-CEACAM1 antibody may be administered to the patient as compared to a patient expressing the wildtype variant of CEACAM1. In some embodiments, the patient is assayed for the presence of a Y34C, a Q44L, and/or a Q89H allelic variant of CEACAM1. In some embodiments, a patient that is found to express a Y34C, a Q44L, an A49V and/or a Q89H allelic variant of CEACAM1 is administered a higher and/or a more frequent dose of an anti-CEACAM1 antibody as compared to a patient expressing the wildtype variant of CEACAM1.

### Pharmaceutical Compositions

In another aspect, the present invention provides pharmaceutically acceptable compositions that comprise a therapeutically effective amount of a CEACAM1 antibody or antigen-binding fragment thereof is described herein formulated together with one or more pharmaceutically acceptable excipients.

The dosage of active agent(s) may vary, depending on the reason for use, the individual subject, and the mode of administration. The dosage may be adjusted based on the subject's weight, the age and health of the subject, and tolerance for the compound(s) or composition. For example, depending on the disease, for an antibody or antigen-binding fragment thereof, this may require 0.1, 1.0, 3.0, 6.0, or 10.0 mg/Kg. For an IgG having a molecular mass of 150,000 g/mole (two binding sites), these doses correspond to approximately 18 nM, 180 nM, 540 nM, 1.08 μM, and 1.8 μM of binding sites for a 5 L blood volume.

The active agent and excipient(s) may be formulated into compositions and dosage forms according to methods known in the art. The pharmaceutical compositions of the present invention may be specially formulated in solid or liquid form, including those adapted for parenteral administration, for example, by subcutaneous, intratumoral, intramuscular or intravenous injection as, for example, a sterile solution or suspension.

Therapeutic compositions comprising antibodies or antigen-binding fragments thereof that bind to CEACAM1 may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as com starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

The compositions comprising an antibody or antigen-binding fragment thereof and a pharmaceutically acceptable carrier may comprise the CEACAM1 antibodies or antigen-binding portions thereof set forth herein at various concentrations. For example, the compositions may comprise an antibody or antigen-binding fragment thereof at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions also may comprise an antibody or antigen-binding fragment thereof at about 10 mg/ml, 20 mg/ml, 30 mg/ml, 40 mg/ml, 50 mg/ml, 60 mg/ml, 70 mg/nil, 80 mg/ml, 90 mg/ml, 100 mg/ml, 110 mg/ml, 120 mg/ml, 130 mg/ml, 140 mg/ml, or 150 mg/ml.

In some embodiments, the compositions comprising the antibody or antigen-binding fragment thereof and the pharmaceutically acceptable carrier are lyophilized and provided in a composition for reconstitution prior to administration.

### Methods of Administration

Therapeutic compositions comprising the contemplated antibody or antigen-binding fragment thereof may be administered in any convenient manner, including by injection, transfusion, implantation or transplantation. The compositions described herein may be administered to a patient subcutaneously, intradermally, intratumorally, intranodally, intramedullary, intramuscularly, intracranially, by intravenous or intralymphatic injection, or intraperitoneally. In one embodiment, the cell compositions of the present invention are preferably administered by intravenous injection.

In certain embodiments, the antibody or antigen-binding fragment thereof is administered to the mammal by intravenous infusion, i.e., introduction of the antibody or antigen-binding fragment thereof into the vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, a dose of a compound or a composition is administered to a subject every day, every other day, every couple of days, every third day, once a week, twice a week, three times a week, once every two weeks, or once a month. In other embodiments, two, three or four doses of a compound or a composition is administered to a subject every day, every couple of days, every third day, once a week, once every two weeks or once a month. In some embodiments, a dose(s) of a compound or a composition is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more.

### Combination Therapies

In one aspect, the invention provides CEACAM1 antibodies or antigen-binding fragments thereof that are administered with an additional therapeutic agent. Such additional agents include, but are not limited to, cytotoxic agents, chemotherapeutic agents, growth inhibitory agents, anti-inflammatory agents, anti-cancer agents, anti-neurodegenerative agents, and anti-infective agents. Agents that are used in such combination therapies may fall into one or more of the preceding categories. The administration of the antibody or antigen-binding fragment thereof and the additional therapeutic agent may be concurrently or consecutively. The administration of the antibody or antigen-binding fragment thereof and the additional therapeutic agent may be separately or as a mixture. Further, the methods of treatment contemplated by the invention can relate to a treatment in combination with one or more cancer therapies selected from the group of antibody therapy, chemotherapy, cytokine therapy, dendritic cell therapy, gene therapy, hormone therapy, laser light therapy, and radiation therapy.

Exemplary additional therapeutic agents also include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary additional therapeutic agents also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary additional therapeutic agents also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary additional therapeutic agents also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g., quinolones, genistein, bactacyclin), busulfan, ifosfamide, mechlorethamine, triaziquone, diaziquone, carbazilquinone, indoloquinone E09, diaziridinyl-benzoquinone methyl DZQ, triethylenephosphoramide, and nitrosourea compounds (e.g., carmustine, lomustine, semustine).

Exemplary additional therapeutic agents also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g. paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)); anti-angiogenesis compounds such as Angiostatin K1-3, DL-α-difluoromethyl-ornithine, endostatin, fumagillin, genistein, minocycline, staurosporine, and (+)-thalidomide.

Exemplary additional therapeutic agents also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary additional therapeutic agents also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary additional therapeutic agents also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary additional therapeutic agents also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other additional therapeutic agents that are compatible with the teachings herein include auristatins (e.g., auristatin E and monomethylauristan E), calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

In one embodiment, the CEACAM antibody or antigen-binding fragment thereof is administered in combination with an agent that is a checkpoint inhibitor. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PD-L1, PD-L2, PD-1, B7-H3, B7-H4, BTLA, HVEM, GAL9, LAG3, TIM-3, VISTA, KIR, 2B4 (belongs to the CD2 family of molecules and is expressed on all NK, γδ, and memory CD8⁺ (αβ) T cells), CD160 (also referred to as BY55), CGEN-15049, CHK1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PD-L1, PD-L2, PD-1, BTLA, HVEM, TIM-3, GAL9, LAG3, VISTA, KIR, 2B4, CD160 and CGEN-15049. Illustrative immune checkpoint inhibitors include Tremelimumab (CTLA-4 blocking antibody), anti-OX40, and Yervoy/ipilimumab (anti-CTLA-4 checkpoint inhibitor), as well as the PD-1 and PD-L1 inhibitors described above. Checkpoint protein ligands include, but are not limited to PD-L1, PD-L2, B7-H3, B7-H4, CD28, CD86 and TIM-3.

In some embodiments, the CEACAM1 antibodies and antigen-binding fragments thereof described herein are administered with a TIGIT, LAP, Podoplanin, Protein C receptor, ICOS, GITR, CD226 or a CD160 inhibiting agent.

In some embodiments, the CEACAM1 antibodies and antigen-binding fragments thereof described herein are administered with a CTLA-4, a PD-1, a PD-L1, or a PD-L2 inhibiting agent. In some embodiments, the CEACAM1 antibodies and antigen-binding fragments thereof described herein are administered with a TIM-3 inhibiting agent.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Generation of Fully Humanized CEACAM1 Antibodies

### 1. Construction of the Chimeric Antibody

A murine anti-CEACAM1 antibody served as the starting point for humanization. The sequencing of the hybridoma expressing the parent antibody identified a single heavy chain sequence and two kappa light chain sequences. In order to determine which light chain sequence encodes the CEACAM1 binding motif, the VH and both Vκ sequences were synthesized with flanking restriction enzyme sites for cloning into the pANT expression vector system for IgG4 (S24IP) heavy and kappa light chain. The VH regions were cloned using MluI and HindIII restriction sites, and the Vκ regions were cloned using BssHII and BamHI restriction sites.

### 2. Expression and Purification of Chimeric Antibody

Combinations of IgG4 (S241P) VH together with the two possible Vκ chains encoding the chimeric parental antibody were transiently transfected into HEK EBNA adherent cells using PEI and incubated for 7 days post-transfection. The combination of VH and one of the Vκ chains failed to express whereas the VH combined with the second Vκ sequence produced significant levels of antibody. The expressed antibody was purified from cell culture supernatant on a Protein A sepharose column, buffer exchanged into PBS pH 7.4 and quantified by OD 280 nm using an extinction coefficient based on the predicted amino acid sequence (Ec_{0.1%}=1.53).

### 3. Competition ELISA Analysis of Chimeric Antibody Binding to Human CEACAM1

The binding of the purified chimeric antibody to human CEACAM1 was assessed in a competition ELISA assay. Nunc Immuno MaxiSorp 96 well flat bottom microtitre plates were pre-coated with 1 μg/ml human GST-CEACAM1 in 1×PBS, overnight at 4° C. The following day the plates were blocked for 1 hour at room temperature with 2% BSA/PBS before washing 2× with PBS/0.05% Tween 20 pH 7.4.

4-fold dilution series of chimeric or irrelevant control IgG4 antibodies starting from 50 μg/ml to 0.068 μg/ml were premixed with a constant concentration of the murine parent antibody (0.15 μg/ml final concentration), added to the plate and incubated for 1 hour at room temperature. Following 3×PBST washes the binding of the murine antibody was detected with anti-mouse-HRP and TMB substrate. The reaction was stopped with 3 M HCl, absorbance read at 450 nm on a Dynex Technologies MRX TC II plate reader.

The expressed chimeric antibody was able to compete with the murine parent antibody for binding to GST-CEACAM1 confirming that the correct light chain had been identified. As expected, the irrelevant human IgG4 did not compete. The heavy (VH₀) and the light (Vκ₀) variable chains of the chimeric antibody are provided as SEQ ID NOs: 10 and 18, respectively.

### 4. Design of Composite Human Antibody Variable Region Sequences

Structural models of the murine parent antibody V regions were produced using Swiss PDB and analyzed to identify important “constraining” amino acids in the V regions that were likely to be essential for the binding properties of the antibody. Most residues contained within the CDRs (using both Kabat and Chothia definitions) together with several framework residues were considered to be important. The VH and Vκ sequences of the murine parent antibody contain typical framework residues and the CDR 1, 2 and 3 motifs are comparable to many murine antibodies. Sequence analysis also revealed a potential N-linked glycosylation motif in both the original mouse hybridoma light chain CDR1 at position 27D (Kabat numbering) and the heavy chain CDR2 at position 56.

Composite human sequences of the parent antibody were created from several human antibodies that were combined to create CDRs similar or identical to those in the murine sequences. For regions outside of, and flanking the CDRs, a wide selection of human sequence segments was identified as possible components of the novel humanized V regions.

### 5. CD4⁺ T Cell Epitope Avoidance

Based upon the structural analysis, a large preliminary set of sequence segments that could be used to create humanized variants were selected and analyzed using iTope™ technology for in silico analysis of peptide binding to human MHC class II alleles (Perry et al 2008) and using the TCED™ of known antibody sequence-related T cell epitopes (Bryson et al 2010). Sequence segments that were identified as significant non-human germline binders to human MHC class II or that scored significant hits against the TCED™ were discarded.

This resulted in a reduced set of segments, and combinations of these were again analyzed, as above, to ensure that the junctions between segments did not contain potential T cell epitopes. Selected sequence segments were assembled into complete V region sequences that were devoid of significant T cell epitopes. Five heavy chains (VH1 to VH5) and four preferred light chain (Vκ1 to Vκ4) sequences were then chosen for gene synthesis, expression in mammalian cells and testing for activity.

### 6. Construction of Humanized Variants

Five humanized VH (VH1 to VH5) and four humanized Vκ sequences (Vκ1 to Vκ4) were synthesized as described for the chimeric antibody as described above and cloned into the pANT expression vector system for IgG4 (S241P) heavy and kappa light chain. See Table 4.

TABLE 4
Heavy and light chains chosen for gene synthesis.

| | |
|---|---|
| Heavy chain | Light chain |

| Variant | SEQ ID No: | Variant | SEQ ID No: |
|---|---|---|---|
| V_{H}0 | 10 | Vκ0 | 18 |
| V_{H}1 | 11 | Vκ1 | |
| V_{H}2 | 12 | Vκ2 | |
| V_{H}3 | | Vκ3 | |
| V_{H}4 | | Vκ4 | 19 |
| V_{H5} | | | |

### 7. Expression and Purification of Humanized Variants

All combinations of composite IgG4 (S241P) VH and Vκ chains (i.e., a total of 20 pairings) plus 2 control antibodies (chimeric VH (VH0) with variant Vκ1 and variant VH1 with chimeric Vκ (Vκ0)), were transiently transfected into HEK EBNA adherent cells using a PEI transfection method and incubated for 5-7 days post-transfection. Antibodies were purified from cell culture supernatants on Protein A Sepharose columns as described above. Antibodies were quantified by OD_{280 nm} using an extinction coefficient based on the predicted amino acid sequence. 1.5 μg of each antibody was analyzed by SDS-PAGE and bands corresponding to the profile of atypical antibody were observed.

### 8. Competition ELISA Analysis of Humanized Variants Binding to CEACAM1

The binding of the purified antibodies to human CEACAM1 were assessed in a competition ELISA assay as described above. The ability of each variant to compete against the murine parental antibody for binding to the GST-CEACAM1 was compared to that of the chimeric antibody (VH0/Vκ0), which was included on each plate.

The competition ELISA data is summarized in Table 5. The binding of variants VH1Vκ1, VH1Vκ2, VH1Vκ3, VH2/Vκ1, VH2Vκ2 and VH2/Vκ3 to CEACAM1 was within 2-fold of the chimeric antibody, therefore these variants were taken forward for Biacore multicycle kinetic analysis.

TABLE 5
Summary of Composite Human Antibody variants and control antibody
titers and binding data. Antibody expression titers (μg/ml)
are from static HEK EBNA transient transfections. IC50 values
obtained in competition assays were normalized to the chimeric
parental antibody tested on the same plate.

| | Average expression | Average relative |
|---|---|---|
| Variant | titer (μg/ml) | IC50 values |
| Chimera | 9.5 | 1 |
| VH0/Vκ1 | 27.1 | 2.5 |
| VH1/Vκ0 | 14.7 | 1.0 |
| VH1/Vκ1 | 24.4 | 1.5 |
| VH1/Vκ2 | 26.5 | 1.3 |
| VH1/Vκ3 | 29.4 | 1.2 |
| VH1/Vκ4 | 26.1 | 4.4 |
| VH2/Vκ1 | 30.6 | 1.7 |
| VH2/Vκ2 | 31.9 | 1.6 |
| VH2/Vκ3 | 33.4 | 1.7 |
| VH2/Vκ4 | 20.3 | 7.2 |
| VH3/Vκ1 | 25.6 | Weak/no binding |
| VH3/Vκ2 | 31.9 | Weak/no binding |
| VH3/Vκ3 | 35.3 | Weak/no binding |
| VH3/Vκ4 | 24.3 | Weak/no binding |
| VH4/Vκ1 | 29.8 | Weak/no binding |
| VH4/Vκ2 | 34.8 | Weak/no binding |
| VH4/Vκ3 | 34.3 | Weak/no binding |
| VH4/Vκ4 | 35.2 | Weak/no binding |
| VH5/Vκ1 | 26.0 | Weak/no binding |
| VH5/Vκ2 | 29.2 | Weak/no binding |
| VH5/Vκ3 | 24.8 | Weak/no binding |
| VH5/Vκ4 | 27.0 | Weak/no binding |

### 9. Kinetic Analysis of Humanized Variants Binding to CEACAM1

As an alternative approach to assess the binding of the antibody combinations and the control antibodies to CEACAM1, a kinetic analysis was performed on a Biacore T200 running Biacore T200 Evaluation Software V2.0.1. All experiments were run at 25° C. with HBS-P+ running buffer (pH 7.4).

All kinetic experiments were performed using CEACAM1-HIS (Sino Biologics Inc.) as the analyte. For all experiments, antibodies were immobilized onto a Series S Protein A sensor chip surface. For kinetic experiments, the amount of immobilized/captured ligand needs to be limited to avoid mass transfer effects at the surface of the chip and the surface should ideally have an analyte binding level (Rₘₐₓ) of 50-150 RUs. Using a MW of 45 kDa for the CEACAM1 analyte, 150 kDa for the antibody ligand (estimated value for IgG), 50 RU for Rmax and the stoichiometry (Sm) as 2 due to the ability of each antibody to bind 2 target molecules, a target response level of 100 RUs was set for capture of all the sample antibodies.

### Single Cycle Analysis

In order to assess the binding of all Composite Human Antibody™ variants, single cycle kinetic analysis was performed on the supernatants of all the transiently transfected HEK EBNA cells. Purified chimeric antibody and purified chimeric antibody spiked into HEK EBNA media were used as positive controls. Supernatants were diluted in HBS-P+ to a concentration of 1 μg/mL (as determined by an IgG quantitation ELISA). At the start of each cycle, antibodies were loaded onto Fc2, Fc3 and Fc4 of a Protein A chip and captured at a flow rate of 8 μL/min to give an RU of ˜100. The surface was then allowed to stabilize. Single cycle kinetic data was obtained at a flow rate of 38 pd/min to minimize any potential mass transfer effects. Multiple repeats of the chimeric antibody were performed to check the stability of the surface and analyte over the kinetic cycles. The signal from the reference channel Fc1 (no antibody) was subtracted from that of Fc2, Fc3 and Fc4 to correct for differences in non-specific binding to a reference surface. A 5 point 2-fold dilution range from 3.125 to 50 nM CEACAM1 without regeneration between each concentration was used. The association phase for the 5 injections of increasing concentrations of CEACAM1 was monitored for 150 seconds and a single dissociation phase was measured for 150 seconds following the last injection of CEACAM1. Regeneration of the Protein A was conducted using 2 injections of 10 mM glycine-HCL pH 1.5 followed by a stabilization period of 500 seconds.

In the raw sensorgrams for the single cycle kinetics, the signal from each antibody blank run (no CEACAM1) was subtracted to correct for differences in surface stability. Single cycle kinetics (Table 6) demonstrated significant kinetic profile differences between the chimeric antibody and humanized variants containing VH3, VH4 and VH5, both in the on and off rates. This change in kinetics correlates well with the inability of these variants to compete effectively with the chimeric antibody in the competition ELISA. Antibody variants containing Vκ4 were also unable to bind CEACAM1, again confirming the results of the competition ELISA.

TABLE 6
Single cycle kinetic data for non-purified Composite Human
Antibody ™ variants and control antibodies binding
to CEACAM1-HIS as determined using the Biacore T200. The
fold difference in KD compared to chimeric parental antibody
was calculated by dividing the KD of the test antibody
variant by that of the spiked chimeric parental antibody.
NB indicates variants that are non-binding.

| | | Relative K_{D} to chimeric |
|---|---|---|
| Variant | K_{D} (nM) | parental antibody |
| VH0/Vκ0 purified | 47.6 | 1.1 |
| VH0/Vκ0 spiked | 43.2 | 1.0 |
| VH0/Vκ1 | 56.1 | 1.3 |
| VH1/Vκ0 | 76.6 | 1.8 |
| VH1/Vκ1 | 93.4 | 2.2 |
| VH1/Vκ2 | 148.0 | 3.4 |
| VH1/Vκ3 | 119.0 | 2.8 |
| VH1/Vκ4 | 143.0 | 3.3 |
| VH2/Vκ1 | 180.0 | 4.2 |
| VH2/Vκ2 | 138.0 | 3.2 |
| VH2/Vκ3 | 72.6 | 1.7 |
| VH2/Vκ4 | 77.8 | 1.8 |
| VH3/Vκ1 | 1500.0 | 34.7 |
| VH3/Vκ2 | 265.0 | 6.1 |
| VH3/Vκ3 | 13400.0 | 310.2 |
| VH3/Vκ4 | NB | NB |
| VH4/Vκ1 | 243.0 | 5.6 |
| VH4/Vκ2 | 3950.0 | 91.4 |
| VH4/Vκ3 | 7060.0 | 163.4 |
| VH4/Vκ4 | NB | NB |
| VH5/Vκ1 | 12100.0 | 280.1 |
| VH5/Vκ2 | 12200.0 | 282.4 |
| VH5/Vκ3 | 185.0 | 4.3 |
| VH5/Vκ4 | NB | NB |

### Multi-Cycle Analysis

The 6 lead variants from the competition ELISA (VH1Nκ1, VH1/Vκ2, VH1/Nκ3, VH2/Vκ1, VH2/Vκ2, and VH2/Vκ₃) were tested by multi-cycle kinetics analysis.

For multi-cycle kinetic analysis purified antibody was immobilized at a protein concentration of 1 μg/mL in HBS-P+. At the start of each cycle, antibody was captured on Protein A to give an RU of ˜100 and the surface allowed to stabilize. Kinetic data was obtained at a flow rate of 35 μl/min to minimize any potential mass transfer effects. Multiple repeats of the blank (no CEACAM1) and a repeat of a single concentration of the analyte were programmed into the kinetic run in order to check the stability of both the surface and analyte over the kinetic cycles. For kinetic analysis, a 2-fold dilution series was selected from 100 to 1.5625 nM CEACAM1. The association phase of CEACAM1 was monitored initially for 180 seconds. In the subsequent experiment a longer association time (275 seconds) was used in order to better reach steady state. The dissociation phase was measured for 250 seconds. Regeneration of the Protein A surface was conducted using 2 injections of 10 mM glycine-HCL pH 1.5 at the end of each cycle.

The signal from the reference channel Fc1 was subtracted from that of Fc2, Fc3 and Fc4 to correct for differences in non-specific binding to a reference surface, a global Rmax parameter was used in the 1-to-1 binding model. The relative K_{D} compared to the chimeric parental antibody was calculated by dividing the K_{D} of the Composite Human Antibody™ variants by that of the chimeric parental antibody on the same chip. Data are summarized in Table 7.

The Composite Human Antibody variants VH1/Vκ1, VH1/Vκ2, VH1/Vκ3, VH2/Vκ1, VH2/Vκ2, and VH2/Vκ3 demonstrated affinity within 2-fold of the chimeric parental antibody. See Table 8.

TABLE 7
The multi cycle kinetic data (n = 1) for six Composite
Human Antibody variants binding to CEACAM1-HIS as determined using
the Biacore T200. The fold difference in K_{D} compared to the chimeric
parental antibody was calculated by dividing the K_{D} of
the test antibody variant by that of the chimeric parental
antibody tested on each chip.

| | | | | | | Relative |
|---|---|---|---|---|---|---|
| | | | | | | K_{D} to |
| | | | | | | chimeric |
| | Kₐ | K_{d} | K_{D} | Rₘₐₓ | Chi² | parental |
| Variant | (M⁻¹s⁻¹) | (s⁻¹) | (nM) | (RU) | (RU²) | antibody |
| VH0/Vκ0 | 1.95 × 10⁵ | 5.30 × 10⁻³ | 27.2 | 50.3 | 0.182 | 1.00 |
| VH1/Vκ1 | 1.01 × 10⁵ | 3.84 × 10⁻³ | 38.1 | 35.1 | 0.041 | 1.40 |
| VH2/Vκ1 | 1.56 × 10⁵ | 5.20 × 10⁻³ | 33.3 | 34.6 | 0.045 | 1.22 |
| VH0/Vκ0 | 2.56 × 10⁵ | 5.71 × 10⁻³ | 22.3 | 45.6 | 0.268 | 1.00 |
| VH1/Vκ2 | 1.95 × 10⁵ | 5.51 × 10⁻³ | 28.3 | 26.3 | 0.0641 | 1.27 |
| VH2/Vκ2 | 1.79 × 10⁵ | 4.96 × 10⁻³ | 27.8 | 30.1 | 0.0835 | 1.25 |
| VH0/Vκ0 | 2.72 × 10⁵ | 5.31 × 10⁻³ | 19.5 | 43.9 | 0.501 | 1.00 |
| VH1/Vκ3 | 2.21 × 10⁵ | 5.94 × 10⁻³ | 26.8 | 28.0 | 0.172 | 1.37 |
| VH2/Vκ3 | 2.11 × 10⁵ | 5.88 × 10⁻³ | 27.8 | 28.0 | 0.170 | 1.43 |

TABLE 8
Summary of relative KD values for 6 tested Composite Human Antibody
variants, obtained from multi cycle kinetics as determined using the
Biacore T200. The fold difference in K_{D} compared to the chimeric parental
antibody was calculated by dividing the K_{D} of the test antibody variant
by that of the chimeric parental antibody tested on each chip. The
average relative K_{D} was determined from two independent experiments.

| | Relative K_{D} to | Relative K_{D} to | Average relative K_{D} to |
|---|---|---|---|
| | chimeric parental | chimeric parental | chimeric parental |
| Variant | antibody expt 1 | antibody expt 2 | antibody |
| VH1/Vκ1 | 1.50 | 1.40 | 1.45 |
| VH1/Vκ2 | 1.66 | 1.27 | 1.46 |
| VH1/Vκ3 | 1.92 | 1.37 | 1.65 |
| VH2/Vκ1 | 1.39 | 1.22 | 1.31 |
| VH2/Vκ2 | 1.29 | 1.25 | 1.27 |
| VH2/Vκ3 | 1.59 | 1.43 | 1.51 |

### Example 2: Removal of N-Linked Glycosylation Sites

Sequence analysis revealed a potential N-linked glycosylation motif in the original mouse hybridoma heavy chain position N56 and within the light chain at position S27 (numbering based upon the Kabat system of numbering). These sites were mutated to remove the N-linked glycosylation sites (i.e., N56Q and S27(f)A). Deglycosylated residues are highlighted in bold in Table 19.

### Example 3: Specificity of Isolated Composite Human Antibody Variants to CEACAM1

Binding selectivity of the selected antibody variants was assessed by flow cytometry using HeLa cells transfected with vectors expressing CEACAM1, CEACAM5, and CEACAM6, respectively. The resulting cells were used for flow cytometry experiments and were cultured at 37° C., 5.0% CO₂ in Dulbecco's modified Eagle's medium supplemented with 10% fetal bovine serum, penicillin (100 U/ml) and dihydrostreptomycin (100 g/ml). Cell lines were stained with the indicated antibodies followed by fluorochrome-conjugated monoclonal antibody specific for the indicated antibody isotypes such as human IgG4 or mouse IgG1 together with viable dye (DAPI). Data were acquired with a Cytoflex flow cytometer (Invitrogen) and analyzed with FlowJo software (TreeStar, V7.6.5 for Windows).

Binding specificities for selected CEACAM1 antibodies are shown in Table 9.

TABLE 9
Binding selectivity of the selected antibody variants was assessed
by flow cytometry using HeLa cells transfected with vectors expressing
CEACAM1, CEACAM5, and CEACAM6, respectively. Indicated is the
relative fluorescence intensity for cells expressing the respective
antigen that the indicated antibody bound to.

| Variant | CEACAM1 | CEACAM5 | CEACAM6 |
|---|---|---|---|
| VH0/VK0 | 84.2 | 0.42 | 2.72 |
| VH1 N56Q/VK4 S27(f)A | 87.1 | 0.0622 | 2.26 |
| VH2 N56Q/VK1 S27(f)A | 88.7 | 3.91 | 13.5 |
| VH2 N56Q/VK2 S27(f)A | 89.4 | 3.04 | 9.5 |
| VH2 N56Q/VK3 S27(f)A | 79.1 | 2.19 | 8.27 |
| VH2 N56Q/VK4 S27(f)A | 89.3 | 0 | 1.31 |
| VH1/VK4 | 81.1 | 4.47 | 1.19 |
| hIgG4 (negative control) | 0 | 0 | 0.0979 |

### Example 4: Expansion of Human Hematopoietic Cells Induced by Antibody Treatment

To assess the efficacy of selected antibodies to stimulate the expansion of CD45⁺ human cells, a HuNSG model was used (Fisher et al. Targeting of 4-1BB by monoclonal antibody PF-05082566 enhances T-cell function and promotes anti-tumor activity. Cancer Immunol Immunother. 2012 October; 61(10):1721-33).

For this, human whole blood was collected from healthy volunteer donors and peripheral blood mononuclear cells (PBMC) isolated. Ten million freshly isolated human PBMCs were adoptively transferred via intraperitoneal injection into NSG (NOD.Cg-Prkdc^{scid} Il2rg^{tm1WjI}/SzJ) host mice. Fifteen days post PBMC injections, animals were administered a single dose of the indicated humanized antibody (2 mg/kg via intraperitoneal injection). After 5 days, PBMCs were stained with antibodies to human CD45 to identify hematopoietic cells and assessed via fluorescence activated cell sorting (FACS). Results are summarized in Table 10. Lower values indicate a higher dilution of the dye inside the cells, and an augmented expansion of the human CD45⁺ cells.

TABLE 10
In vivo treatment of human CD45⁺ cells by selected CEACAM1
antibodies induces their proliferation. Lower values indicate
a higher dilution of the dye inside the cells that is indicative
of human hematopoieitic CD45⁺ cell expansion.

| Antibody | Fluorescence intensity |
|---|---|
| hIgG4 control antibody | 126,218 |
| VH0/Vκ0 | 100,419 |
| VH2/Vκ4 | 62,015 |
| VH1/Vκ4 | 38,087 |

### Example 5: Affinity Maturation of VH2 N56Q/Vκ4 S27(f)A scFv

1. Phage Vector Construction and Testing of Binding of Parent VH2 N56Q/Vκ4 S27(f)A scFv

The template for affinity maturation was the VH2 N56Q/Vκ4 S27(f)A aglycosylated humanized antibody. Genes encoding the starting antibody VH and Vκ were converted to a scFv format using overlap PCR where the heavy chain was linked to the light chain via a 15 amino acid (G4S)₃ linker (SEQ ID NO:47). The scFv sequence was then cloned into the phagemid vector pANT65 using the restriction enzymes NcoI and NotI, allowing for display of scFv on the phage surface as a gene III fusion protein. This plasmid also allows expression of a C-terminal Flag tag on the expressed scFv. The cloned scFv was transformed into E. coli (TG1) and all constructs were confirmed by sequencing.

M13 helper phage (monovalent) and hyper phage (multivalent) displaying either the parent VH2 N56Q/Vκ4 S27(f)A scFv or an irrelevant scFv were prepared and tested for binding to CEACAM1 (Sino Biological). Phage derived from the parent VH2 N56Q/Vκ4 S27(f)A sequence bound specifically to the antigen with no binding observed with the irrelevant phage. As expected, parent scFv hyper phage bound at lower titer than parent M13 helper phage as a result of presentation of an increased number of scFv on the phage surface.

### 2. Library Design

The major determinants of antigen binding within an antibody are the CDRs (Wu and Kabat, 1970). Targeting mutations to VH CDR3 and Vκ CDR3 has been shown to be one of the most effective means of improving affinity by phage display (e.g., Shier et al, 1996).

For the construction of an affinity maturation library, specific amino acids within the VH CDR3 and Vκ CDR3 of the humanized antibody were targeted for “hotspot” mutagenesis using semi-randomized codons. Sequence positions were analyzed for likely contact residues (http://www.bioinf.org.uk/abs/MeanContacts.html) and ranked in order within each block. This information was used together with the amino acid preferences at any given position within the heavy chain and light chain CDR3 (http://www.abysis.org/). Where possible, priority was given to the highest ranked contact residue within each block.

The VH CDR3 was identified as being 11 amino acids in length (Kabat definition) and was split into two libraries overlapping at G99: Block 1 (G95 to G99) and Block 2 (G99 to A101), with each containing a subset of amino acids in all positions. Position T93 was included in Block 1 to allow more diversity in the germline residue anchoring the CDR. Vx CDR3 (Q90 to W96) was covered in a single library with a subset of amino acids included at each position. Kabat numbering is used for all protein sequence coordinates.

### 3. Library Construction

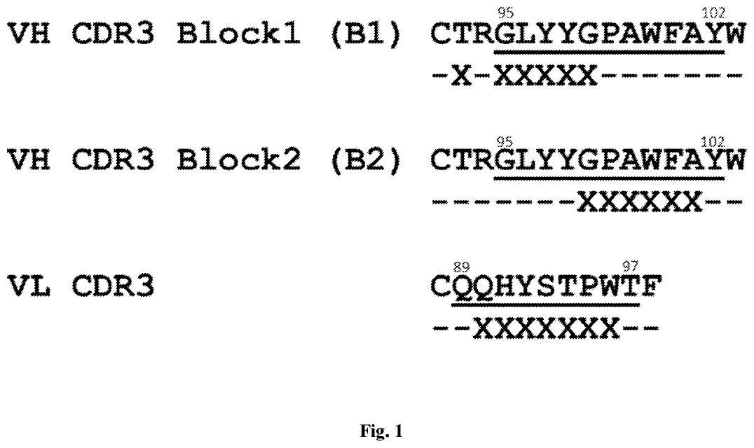

All oligos were designed as described previously and synthesized (IDT, Integrated DNA Technologies). Degenerate primers were hand mixed to reduce nucleotide biases during synthesis. An overview of library construction is shown in FIG. 1.

Non-expressing plasmids containing VH2 N56Q/Vκ4 S27(f)A parental sequences configured as a scFv were designed to contain stop codons to prevent the parent scFv from dominating selections (as is often observed for affinity maturation programs). Stop codons were placed in the VH2 N56Q/Vκ4 S27(f)A scFv in pANT65 using Quikchange mutagenesis (Agilent) in each of the areas of sequence to be randomized, such that only mutated antibody variants where the stop codons had been replaced by library encoded amino acids were expressed. By using this approach, non-mutated parent sequences would be truncated and therefore would not be displayed.

For the two VH CDR3 libraries (Block 1 and Block 2), randomization was carried out by performing two PCRs using a stop codon template. Initially, a portion of the VH together with the Vκ was amplified with the randomized 5′ library primer and a 3′ primer specific for the Vκ light chain FW4. In a separate PCR, the remainder of the VH was amplified with a 5′ primer based in the heavy chain FW1 region plus a 3′ primer that was complementary to a portion of the VH CDR randomized primer. The full length VH CDR randomized scFv libraries were then constructed by annealing of the two amplified fragments and re-amplification of the scFv by PCR with primers that appended two restriction sites (either Nco I or Not I) for subcloning of the fragment.

For the Vκ CDR3 library, randomization of the Vκ CDR3 was carried out by performing two PCRs. In the first PCR, a randomized 3′ primer and a V_{H} FW1 specific 5′ primer containing a Nco I restriction site were used to amplify the majority of the scFv gene and introduce mutations into the Vκ CDR3. The second PCR added the remainder of the scFv and appended a restriction site (Not I) for subcloning of the fragment.

Purified, amplified DNA for all three libraries was digested using Nco I and Not I and ligated into the similarly cut phagemid vector (pANT65). Ligated DNA was precipitated, resuspended in nuclease-free water and transformed by electroporation into freshly prepared electrocompetent TG1 cells. The following day, colonies were counted, plates scraped, and glycerol stocks prepared. Libraries were electroporated multiple times in order to sufficiently cover the theoretical library diversity. The observed library size of each of the three libraries is shown in Table 11. In all cases, a coverage of 4.95-fold or greater was obtained. Individual colonies from each of the three libraries were sequenced to confirm that the appropriate CDR block had been mutated.

TABLE 11
Theoretical and observed phage library sizes
for the three CDR libraries prepared.

| | VH CDR3 | VH CDR3 | |
|---|---|---|---|
| | Block 1 | Block 2 | Vκ CDR3 |
| | Library | Library | Library |
| Theoretical Diversity (based on | 2.52 × 10⁷ | 2.52 × 10⁷ | 2.83 × 10⁷ |
| library DNA sequence) | | | |
| Observed Colony Count | 1.25 × 10⁸ | 1.79 × 10⁸ | 2.02 × 10⁸ |
| Library Coverage | 4.95 | 7.11 | 7.12 |

### 4. Library Phage Rescue

Bacteria from each library were inoculated into 150 ml 2TYCG (2%) cultures using inocula at least 100× the observed library diversity. The cultures were grown to mid-log phase (OD_{600 nm}≈0.4) and the total number of cells estimated (based on an OD_{600 nm} of 1≈5×10⁸ cells/ml). Hyper phage was added and incubated for 15 min (static) followed by 45 min at 250 rpm, then centrifuged, resuspended in 2TYCK media and grown overnight at 30° C. The following day, phage were harvested by recovering the culture supernatant by centrifugation followed by precipitation using 3/10th volume of chilled 20% PEG/2.5 M NaCl. After 1 hour incubation on ice, precipitated phage were recovered by centrifugation and the pellet resuspended in 1×PBS pH 7.4. The supernatant was re-centrifuged to remove any cellular debris, following which the supernatant was re-precipitated as described above. The precipitated phage were resuspended in 1×PBS pH 7.4. To increase the chances of obtaining scFvs with increased affinity, multivalent hyper phage (M13 K07ΔpIII helper phage) were used at a multiplicity of infection of 20 for library rescue due to the relatively low affinity of the starting antibody. Following the first round of selection, monovalent M13K07 helper phage (New England BioLabs) at a multiplicity of infection of 10 were used as a result of an expected enrichment of antigen binders.

### 5. Affinity Improved Phage Selection

CEACAM1 was used throughout for positive selections. Soluble deselections using the closely related family members CEACAM3. CEACAM5 and CEACAM6 (Sino Biological) were performed at a final concentration of 50 nM of each protein to reduce the possibility of selecting affinity matured scFvs with CEACAM3, CEACAM5 and CEACAM6 cross reactivity. This was performed twice during the campaign, by deselecting prior to round two and three. Throughout the campaign the three libraries were kept separate at all stages.

For the selections, each of the libraries were pre-blocked with 3% BSA/PBS following which phage were incubated with decreasing concentrations of biotinylated soluble CEACAM1 antigen for up to two hours. Following incubation, streptavidin paramagnetic beads (pre-blocked as above) were added to each selection and rotated turning end-over-end for 10 minutes. Streptavidin-antigen-phage complexes were washed using increasing numbers of washes with PBST at each successive round of selection followed by a PBS wash, capturing with a magnet between each step. For the VH B2 library, in round two, a higher concentration of CEACAM1 was used to enrich the number of phage binders selected due to the low round one output titer. For the light chain library only, a fourth round of selections was performed to further enrich the positive binding phage as the round three output titers were relatively large. Phage were eluted from the beads by the addition of 50 mM HCl following which the solution was neutralized by the addition of 1 M Tris-HCl pH 9.0.

6. Expression of Soluble scFv (Periprep)

Soluble scFv were initially expressed and tested as crude periplasmic extracts. Individual colonies from selection outputs were picked into 1 ml 2TYCG (0.1%) media and grown by shaking at 37° C. for 5 hours. Cultures were induced by adding IPTG to a final concentration of 1 mM and then grown overnight, with shaking, at 30° C. The following day, cultures were centrifuged, and the supernatant discarded. Bacterial pellets were resuspended in TES buffer pH 7.4 and incubated on ice for 30 minutes. The plate was then centrifuged and the scFv-containing supernatant transferred to a fresh plate for assay.

### 7. High Throughput Competition Screening of Peripreps

Peripreps of colonies from different rounds of selection were screened in a single point competition assay for their ability to compete with parental RSB02 IgG for binding to CEACAM1. Parental RSB02 scFv and an irrelevant scFv were included on each assay plate for comparison.

Nunc Immuno MaxiSorp 96 well flat bottom microtitre plates were coated with CEACAM1 at 0.0625 μg/ml overnight. Plates were subsequently washed and blocked for 1 hour at room temperature with 3% BSA/PBS. Periplasmic extracts were diluted 1:1 with VH2 N56Q/Vk4 S27(f)A IgG (parental RSB02 IgG) at 0.246 μg/mL in 6% BSA/PBS. The diluted periplasmic extract was then added to the blocked plate (100 μL per well) and the samples incubated on the plate for 1 hour at room temperature. Plates were subsequently washed and the binding of VH2 N56Q/Vk4 S27(f)A IgG was detected with an anti-human-HRP antibody (Sigma, Gillingham, UK) and TMB substrate (Invitrogen). The reaction was stopped with 1 M HCl, absorbance read at 450 nm on a Dynex Technologies MRX TC II plate reader and the binding data plotted.

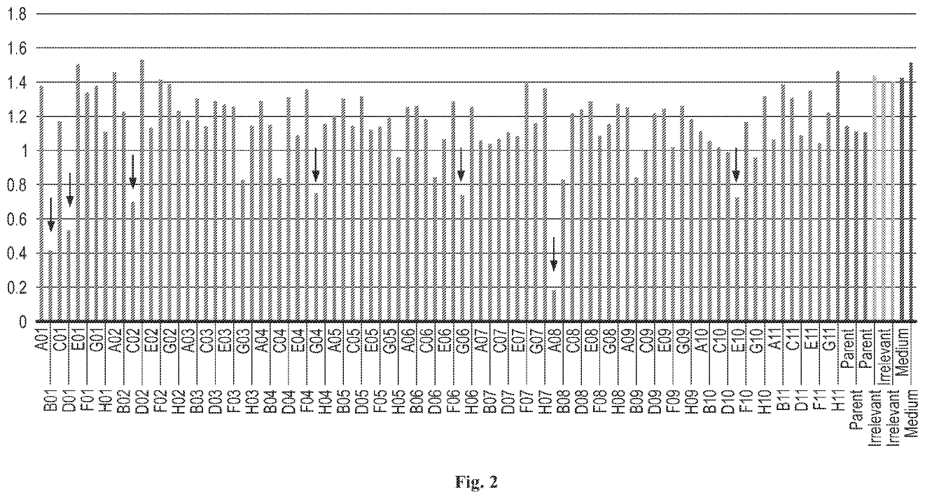

Improved clones were identified on the basis of activity in the competition ELISA relative to the parental RSB02 scFv assayed on the same plate. Greater than 7300 peripreps were analyzed in total from all three libraries. Positive hits were picked into ‘cherry’ plates, re-tested within the competition ELISA to confirm results and sequenced. Dominant single sequence clones were identified in both the VH B1 (˜41% of clones sequenced after round 3 were a single dominant clone) and VL libraries (˜20% of clones were single dominant clones from those sequenced from round 3 and 4). The VL library appeared to contain scFvs with a greater ability to inhibit, resulting in a higher (at least 50% inhibition) cut off point for a positive hit. See FIG. 2.

To select the lead VH and VLs for combining to produce IgGs, positive hits (from primary and secondary screens) were ranked to determine those with the highest average percentage inhibition compared to parent. Five VH B1 clones, three VH B2 clones and 11 VL clones were selected. During sequence analysis, L4 was identified as a mixed VL clone consisting of two different sequences. These were cloned separately to generate the clones L4 #1 and L4 #2 resulting in a total of twelve VL clones being taken forward. Following reformatting, light chain L4 #2 was subsequently shown to have the same sequence as L17. He3 which originally came from the VH B1 library in addition to containing changes within B1 also contained a single mutation in B2. A summary of the twenty lead clones with their CDR sequences is shown in Table 12.

TABLE 12
Summary of the 20 scFv variant leads identified using the CEACAM1 competition ELISA.
Parent CDR shown at the top of the table. The VH B1 and B2 and Vκ regions targeted in
the parent sequence are shown in bold. The mutations differing from the parent sequence
within VH CDR3 B1, VH CDR3 B2 and Vκ CDR3 for each clone are underlined. Note: L4#2
and L17 were shown to be the same sequence.

| | Library | | | | | |
|---|---|---|---|---|---|---|
| | VH B1 | VH B1 | VH B2 | VH B2 | VL | VL |
| Clone | Sequence | SEQ ID NO | Sequence | SEQ ID NO | Sequence | SEQ ID NO |
| Parent | CTRGLYYGPAWFAY | 27 | CTRGLYYGPAWFAY | 27 | QQHYSTPWT | 36 |
| He3 | CTRALTYGPAWLAY | 28 | | | | |
| He4 | CTRALYYGPAWFAY | 29 | | | | |
| He6 | CARARTYGPAWFAY | 30 | | | | |
| He9 | CARAMYYGPAWFAY | 31 | | | | |
| He17 | CTRARTYGPAWFAY | 32 | | | | |
| He1 | | | CTRGLYYGPSWFAY | 33 | | |
| He13 | | | CTRGLYYGPSWLAY | 34 | | |
| He14 | | | CTRGLYYGPSWVAY | 35 | | |
| L1 | | | | | QSHYPFYYT | 8 |
| L2 | | | | | QSHYPFPYT | 37 |
| L3 | | | | | QSHFPFPFT | 38 |
| L4 #1 | | | | | QSHFPYYYT | 39 |
| L4 #2 | | | | | QSHYPYPYT | 40 |
| L5 | | | | | QSHYPFAFT | 41 |
| L6 | | | | | QSHYPFSFT | 42 |
| L16 | | | | | QSHYPYPFT | 43 |
| L17 | | | | | QSHYPYPYT | 44 |
| L18 | | | | | QSHFPYPFT | 45 |
| L19 | | | | | QSHIYPNFT | 46 |
| L20 | | | | | QSHFPYPLT | 9 |

### Example 6: Construction and Testing of Affinity Matured Whole Antibodies

1. Reformatting of Lead scFvs into Antibodies

The twenty variants identified by scFv screening were PCR amplified using primers that introduced flanking restriction enzyme sites for cloning into the IgG4 S241P pANTVhG4 vector and kappa light chain pANTVK vector. Eight affinity matured VH variants were subcloned into the IgG4 S241P pANTVhG4 vector using MluI and HindIII restriction sites. Similarly, twelve affinity matured Vκ sequences were subcloned into the Kappa light chain pANTVK vector using BssHII and BamHI restriction sites.

2. Expression of the Lead 20 Reformatted scFvs as Whole IgGs

For expression, the eight lead humanized affinity matured IgG4 VH variants together with the parent humanized heavy chain (VH2 N56Q) were combined with the parent humanized light chain (Vκ4 S27(f)A) and the 12 lead humanized affinity matured kappa light chains to give a total of 117 combinations. These combinations were transiently transfected into HEK EBNA adherent cells (LGC Standards) in 12-well plates using a PEI transfection method. Seven days post-transfection. the supernatants were harvested, quantified by ELISA and filtered for Biacore single cycle kinetics analysis.

### Example 7: Single Cycle Kinetics Analysis of Humanized and Affinity Matured Lead IgGs Binding to CEACAM1

To assess the binding of the humanized affinity matured IgGs, single cycle kinetics analysis was performed on crude supernatants using a Biacore T200 running Biacore T200 Control Software V2.0.1 and Biacore T200 Evaluation Software V3.0 (Uppsala, Sweden). Antibodies were diluted in HBS-P+ (GE Healthcare) to a final concentration of 1.0 g/ml. At the start of each cycle, antibodies were loaded onto Fc2, Fc3 and Fc4 of the Protein A chip (GE Healthcare). IgGs were captured at a flow rate of 10 μl/min to give an immobilization level (RL) of ˜100 RU (a level calculated to obtain an Rmax of ˜50-150 RU once the analyte is bound). The surface was then allowed to stabilize. Single cycle kinetics data was obtained with CEACAM1 as the analyte at a flow rate of 35 l/min to minimize any potential mass transfer effects. Multiple repeats with the parent (VH2 N56Q/Vκ4 S27(f)A) antibody were performed to check the stability of the surface and analyte over the kinetic cycles. The signal from the reference channel Fc1 (no antibody) was subtracted from that of Fc2, Fc3 and Fc4 to correct for differences in non-specific binding to the reference surface. A three point, two-fold dilution range from 25 nM to 100 nM CEACAM 1 without regeneration between each concentration was used. The signal from each antibody blank run (no CEACAM1) was subtracted to correct for differences in surface stability. The association phase for the three injections of increasing concentrations of CEACAM1 was monitored for 200 seconds each time and a single dissociation phase was measured for 300 seconds following the last injection of CEACAM1. Regeneration of the Protein A surface was conducted using two injections of 10 mM glycine-HCL pH 1.5 followed by a stabilization period of 240 seconds.

The 117 combinations were tested in two experiments. Table 13. Single-cycle kinetic constants demonstrated that all affinity matured antibodies bound to CEACAM.

TABLE 13
Single-cycle kinetics analysis of humanized and
affinity matured lead IgGs binding to CEACAM1.

| Variant | K_{D} | Fold |
|---|---|---|
| He9/L4#2 | 57 | 9.71 |
| He9/L6 | 22 | 7.10 |
| He9/L3 | 27 | 5.85 |
| He9/L16 | 32 | 5.05 |
| He9/L17 | 33 | 4.88 |
| He9/L18 | 36 | 4.40 |
| He4/L4#2 | 127 | 4.34 |
| He3/L4#2 | 130 | 4.24 |
| Parent VH/L1 | 41 | 3.87 |
| He6/L4#2 | 146 | 3.78 |
| He9/L20 | 44 | 3.61 |
| He9/L5 | 44 | 3.61 |
| Parent VH/L4#2 | 163 | 3.38 |
| He4/L16 | 52 | 3.04 |
| He4/L18 | 55 | 2.89 |
| He4/L3 | 55 | 2.87 |
| Parent VH/L18 | 56 | 2.83 |
| Parent VH/L6 | 57 | 2.81 |
| Parent VH/L16 | 61 | 2.62 |
| He3/L1 | 62 | 2.57 |
| He4/L17 | 62 | 2.55 |
| Parent VH/L3 | 63 | 2.53 |
| He4/L1 | 64 | 2.49 |
| Parent VH/L5 | 66 | 2.42 |
| He6/L16 | 66 | 2.41 |
| He17/L16 | 67 | 2.36 |
| He4/L5 | 68 | 2.36 |
| He9/L1 | 69 | 2.32 |
| He14/L1 | 69 | 2.30 |
| He9/L2 | 70 | 2.27 |
| Parent VH/L2 | 70 | 2.26 |
| He4/L6 | 72 | 2.22 |
| He3/L16 | 72 | 2.22 |
| He3/L20 | 74 | 2.15 |
| He17/L4#2 | 258 | 2.14 |
| He1/L3 | 263 | 2.10 |
| He4/L2 | 76 | 2.09 |
| He1/L20 | 264 | 2.09 |
| Parent VH/L17 | 76 | 2.08 |
| Parent VH/L19 | 77 | 2.08 |
| He9/L4 #1 | 78 | 2.05 |
| He17/L1 | 78 | 2.03 |
| He3/Parent VL | 78 | 2.03 |
| He17/L6 | 79 | 2.01 |
| He17/L5 | 81 | 1.97 |
| He17/L2 | 82 | 1.95 |
| He3/L18 | 82 | 1.94 |
| He6/L18 | 83 | 1.92 |
| He3/L17 | 83 | 1.91 |
| He4/L4 #1 | 83 | 1.91 |
| He13/L4#2 | 289 | 1.91 |
| He6/L5 | 84 | 1.89 |
| He3/L2 | 86 | 1.85 |
| He1/L4#2 | 301 | 1.83 |
| He14/L19 | 87 | 1.83 |
| Parent VH/L20 | 87 | 1.83 |
| He14/Parent VL | 88 | 1.80 |
| He1/L5 | 307 | 1.80 |
| He6/L20 | 89 | 1.79 |
| He6/L6 | 89 | 1.78 |
| He4/L19 | 90 | 1.77 |
| He4/L20 | 90 | 1.77 |
| He6/L17 | 91 | 1.75 |
| He14/L18 | 91 | 1.75 |
| He13/Parent VL | 91 | 1.74 |
| He3/L6 | 93 | 1.72 |
| He14/L4 #1 | 93 | 1.70 |
| He14/L16 | 93 | 1.70 |
| He1/L18 | 326 | 1.69 |
| He6/L3 | 94 | 1.69 |
| He17/L3 | 95 | 1.68 |
| He3/L5 | 95 | 1.68 |
| He13/L5 | 95 | 1.68 |
| He14/L17 | 97 | 1.64 |
| He17/L18 | 98 | 1.62 |
| He6/L1 | 98 | 1.62 |
| He1/L16 | 341 | 1.62 |
| He13/L2 | 98 | 1.62 |
| He17/L20 | 99 | 1.61 |
| He3/L4 #1 | 99 | 1.61 |
| He14/L2 | 102 | 1.56 |
| He14/L5 | 102 | 1.56 |
| He13/ | 103 | 1.54 |
| L18 | | |
| Parent | 103 | 1.54 |
| VH/L4 | | |
| #1 | | |
| He17/L17 | 104 | 1.53 |
| He6/L2 | 104 | 1.53 |
| He13/L6 | 105 | 1.51 |
| He13/L16 | 105 | 1.51 |
| He14/L3 | 107 | 1.49 |
| He13/L17 | 372 | 1.48 |
| He1/L17 | 374 | 1.47 |
| He13/L3 | 108 | 1.47 |
| He13/ | 109 | 1.46 |
| L20 | | |
| He14/L6 | 111 | 1.43 |
| He14/ | 114 | 1.39 |
| L20 | | |
| He14/ | 396 | 1.39 |
| L4#2 | | |
| He13/L4 | 115 | 1.38 |
| #1 | | |
| He1/L6 | 402 | 1.37 |
| He1/L4 | 429 | 1.28 |
| #1 | | |
| He6/ | 125 | 1.27 |
| Parent VL | | |
| He13/L1 | 126 | 1.26 |
| He17/ | 128 | 1.24 |
| Parent VL | | |
| He13/ | 129 | 1.23 |
| L19 | | |
| He17/ | 135 | 1.18 |
| L19 | | |
| He3/L19 | 135 | 1.18 |
| He1/L2 | 487 | 1.13 |
| He4/ | 155 | 1.03 |
| Parent VL | | |
| He6/L4 | 155 | 1.03 |
| #1 | | |
| He1/ | 544 | 1.01 |
| Parent VL | | |
| He13/L17 | 159 | 1.00 |
| Parent | 159 | 1.00 |
| VH/ | | |
| Parent VL | | |
| Parent | 551 | 1.00 |
| VH/ | | |
| Parent VL | | |
| He3/L3 | 163 | 0.98 |
| He1/L1 | 568 | 0.97 |
| He17/L4 | 165 | 0.96 |
| #1 | | |
| He9/L19 | 193 | 0.82 |
| He6/L19 | 237 | 0.67 |
| He9/ | 262 | 0.61 |
| Parent VL | | |
| He1/L19 | 996 | 0.55 |

Single cycle kinetics data achieved for 117 humanized and affinity matured variants tested supernatants, ranked from best to worst.
Fold = Fold better than Parent.
K_{D} is in nM. Variants chosen for further analysis are shown in bold.

Following analysis of the single-cycle data, twelve variants were chosen based on improvements in affinity together with observed differences in the kinetic profile including an apparent faster association and dissociation rate observed with He3/L20, He17/L1 and He17/L16. The twelve variants included multiple heavy and light chains with varying fold improvements to mitigate the risk of cross-reactivity to other CEACAM family members (bold in Table 13).

### Example 8: Testing of Lead Antibodies

### 1. Expression, Purification, and Testing of Lead Antibodies

The 12 lead combined variants together with parent IgG (variants indicated in bold in Table 13) were transiently transfected into HEK EBNA adherent cells in triple flasks using the PEI method and incubated for 5-7 days post-transfection. Antibodies were purified from cell culture supernatants on Protein A sepharose columns (GE Healthcare), buffer exchanged into PBS pH 7.2 and quantified by OD_{280 nm} using an extinction coefficient based on the predicted amino acid sequence. 2 μg of each reduced antibody was analyzed by SDS-PAGE and bands corresponding to the profile of a typical antibody were observed.

### 2. Cross-Reactivity ELISA on 12 Lead Variants for Binding to CEACAM1/-3/-5/-6

Binding experiments using Parent IgG had previously demonstrated specific binding to CEACAM1 with no cross-reactivity to CEACAM3/5 and 6. The cross-reactivity to CEACAM3/5 and 6 of the 12 lead variants and Parent IgG was assessed using a binding ELISA assay. Binding to CEACAM1 within the same assay was used as a positive control. The murine pan-CEACAM antibody D14HDT 1 (Abcam) was used as a positive control to confirm the integrity of CEACAM1-/-3/-5 and -6.

Nunc Immuno MaxiSorp 96 well flat bottom microtitre plates (ThermoFisher) were pre-coated with 0.5 μg/ml CEACAM1 in 1×PBS, overnight at 4° C. The following day the plates were blocked for 1 hour at room temperature with 2% BSA/PBS before washing 2× with PBS/0.05% Tween 20 pH 7.4. In order to identify suitable antibody concentrations to assess cross reactivity a binding curve of the parent IgG and D14HD1 was generated. A three-fold dilution series was performed starting from 5.0 μg/ml to 0.0069 μg/ml; these were added to the plate and incubated for 1 hour at room temperature. Following 3×PBST washes, wells containing parent IgG were incubated with goat anti-human IgGκ chain-HRP antibody (Merck Millipore). Wells containing D14HD11 were incubated with anti-mouse-peroxidase IgG (Sigma Aldrich) Binding was detected using TMB substrate (Invitrogen) and the reaction was stopped with 3 M HCl. The absorbance was read at 450 nm on a Dynex Technologies MRX TC II plate reader and this was used to plot binding curves. A full sigmoidal curve was obtained for both the RSB02 parent antibody and D14HD11.

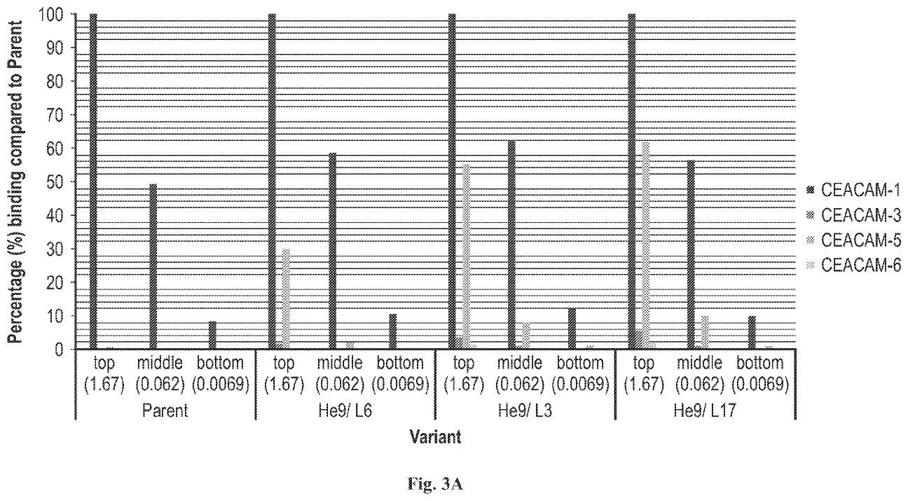

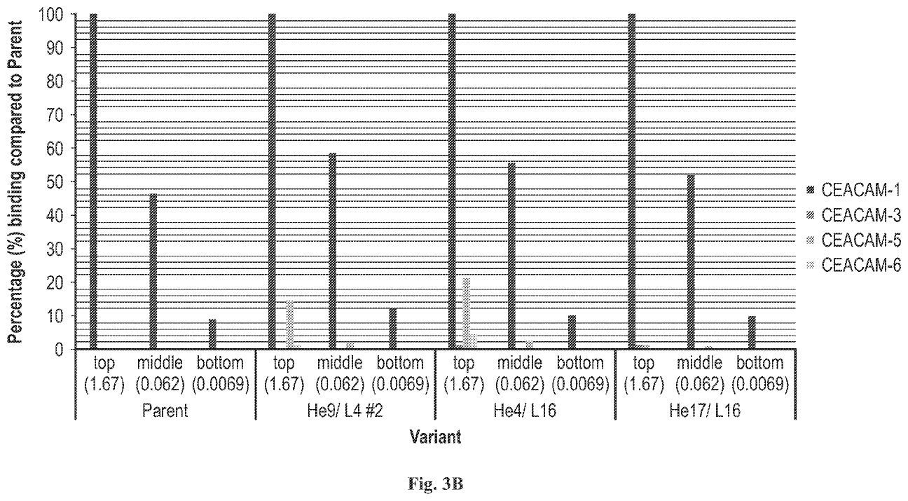

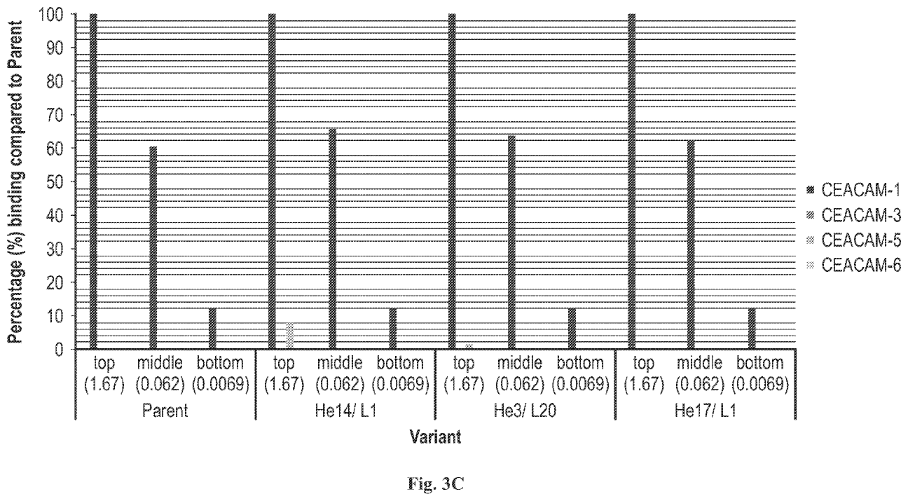

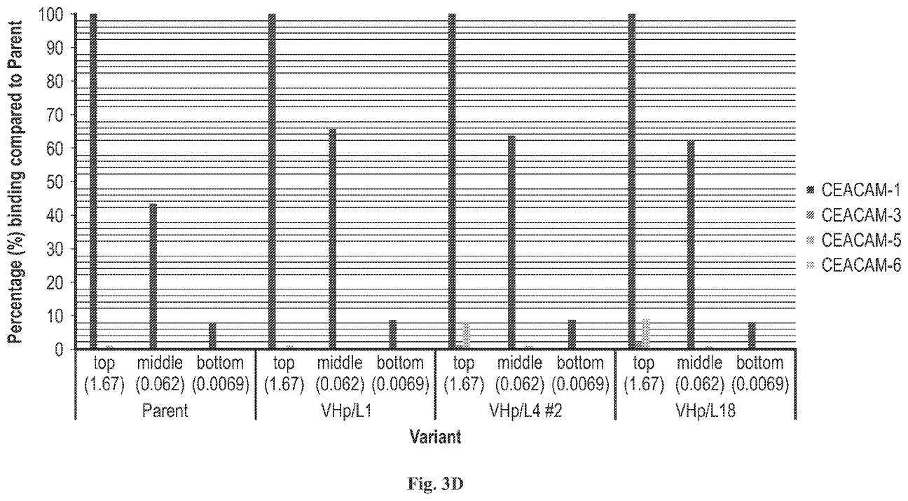

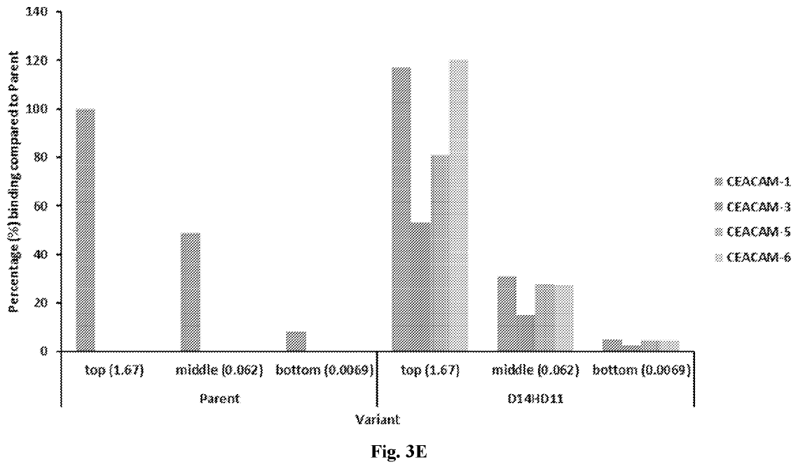

In order to test cross reactivity, three concentrations were chosen for testing corresponding to the top (1.67 μg/ml), middle (0.062 μg/ml) and a bottom (0.0069 μg/ml) points of the binding curve generated for parent IgG CEACAM1 binding. All 12 variants were tested at 1.67 μg/ml, 0.062 μg/ml and 0.0069 μg/ml for binding to CEACAM1/-3/-5 and -6, all coated at 0.5 μg/ml in 1×PBS, overnight at 4° C. The absorbance of the parent IgG binding to CEACAM1 at 1.67 μg/ml was set at 100% binding. The percentage of CEACAM1/-3/-5 and -6 binding for all variants was then calculated relative to the absorbance for CEACAM1 binding of parent IgG at 1.67 μg/ml on the same plate. The plotted binding data achieved for the 12 variants are shown in FIG. 3.

As expected, RSB02 parent IgG and all 12 variants tested bound to CEACAM1 at all concentrations tested (FIG. 3). As a positive control, D14HD11 was tested and shown to bind CEACAM1/-3/-5 and -6 (FIG. 3E).

This high degree of selectivity can be observed despite the fact that the N-domains of different CEACAM share high degrees of homology: The N-domains of CEACAM1 and CEACAM3 are 88% identical, the N-domains of CEACAM1 and CEACAM5 are 89% identical, and N-domains of CEACAM1 and CEACAM6 are 90% identical, as indicated by a percent identity matrix created using Clustal2.1. See FIG. 13 of PCT publication WO2020/118295.

### 3. Cross Reactivity Analysis of 12 Leads by Single-Cycle Kinetics

To assess the cross reactivity of the humanized, affinity matured lead IgGs further, single-cycle Biacore kinetics analysis was performed using CEACAM3, 5 and 6 together with CEACAM1 as a positive control.

Antibodies were diluted in HBS-P+ to a final concentration of 1.0 g/ml. At the start of each cycle, antibodies were loaded onto Fc2, Fc3 and Fc4 of the Protein A chip. IgGs were captured at a flow rate of 10 μl/min to give an Rmax of 50-150 following CEACAM binding. Multiple repeats with the Parent (VH2 N56Q/Vκ4 S27(f)A) antibody were performed to check the stability of the surface and analyte over the kinetic cycles. The signal from the reference channel Fc1 (no antibody) was subtracted from that of Fc2, Fc3 and Fc4 to correct for differences in non-specific binding to the reference surface. A four point, two-fold dilution range from 25 nM to 200 nM CEACAM1/-3/-5/-6 without regeneration between each concentration was used. The signal from each antibody blank run (no CEACAM) was subtracted to correct for differences in surface stability. The association phase for the four injections of increasing concentrations of each CEACAM was monitored for 275 seconds each time and a single dissociation phase was measured for 250 seconds following the last injection of each CEACAM. Regeneration of the Protein A surface was conducted using two injections of 10 mM glycine-HCL pH 1.5 followed by a stabilization period.

The murine Pan-CEACAM antibody D14HDT 1 was used as a positive control to determine functionality of CEACAM1/-3/-5/-6. Using a Protein A sensor chip lead to an unsatisfactory capture of D14HDT 1 therefore an anti-mouse capture kit (GE Healthcare) was used and an additional run performed. D14HD11 was diluted to 5 μg/ml in HBS P+ and loaded onto Fc2 of the anti-mouse sensor chip at a flow rate of 10 μl/min to give the immobilization levels required for each CEACAM. The signal from the reference channel Fc1 (no antibody) was subtracted from that of Fc2 to correct for differences in non-specific binding to the reference surface. A three point, two-fold dilution range from 70 nM to 280 nM CEACAM1/-3/-5/-6 without regeneration between each concentration was used. The signal from each antibody blank run (no CEACAM) was subtracted to correct for differences in surface stability. The association phase for the three injections of increasing concentrations of CEACAM was monitored for 275 seconds each time and a single dissociation phase was measured for 1800 seconds (30 minutes) following the last injection of CEACAM. Regeneration of the anti-mouse sensor surface was conducted using one injection of 10 mM glycine-HCL pH 1.7 followed by a stabilization period

While all the variants tested bound to CEACAM1, no binding to CEACAM3/5/6 was observed by Biacore. This is consistent with the binding observed with the parent IgG. D14HD11 showed binding to all CEACAMs tested.

### 4. Multi-Cycle Kinetics of 12 Lead Variants on CEACAM1

Multi-cycle kinetics analysis was performed on the twelve lead antibodies using a Biacore T200 instrument running Biacore T200 Evaluation Software V3.0.1 (Uppsala, Sweden) to establish an accurate affinity for CEACAM1. The purified antibodies were diluted to a concentration of 1 g/ml in HBS-P+. At the start of each cycle, each antibody was captured on the Protein A surface to give an RL of ˜100 RU. Following capture, the surface was allowed to stabilize. Kinetic data was obtained using a flow rate of 35 l/min to minimize any potential mass transfer effects. For the kinetics analysis, CEACAM1 was used. Multiple repeats of the blank (CEACAM1) and a repeat of a single concentration of the analyte were programmed into the kinetic run in order to check the stability of both the surface and analyte over the kinetic cycles. For kinetic analysis, a two-fold dilution range was selected from 200 to 3.125 nM CEACAM1. The association phase of CEACAM1 was monitored for 275 seconds and the dissociation phase was measured for 250 seconds. Regeneration of the Protein A surface was conducted using two injections of 10 mM glycine-HCL pH 1.5 at the end of each cycle.

The signal from the reference channel Fc1 was subtracted from that of Fc2, Fc3 and Fc4 to correct for differences in non-specific binding to a reference surface, and a global Rmax parameter was used in the 1-to-1 binding model. The relative K_{D} was calculated by dividing the K_{D} of the RSB02 Parent by that of the humanized and affinity matured variants on the same chip. The kinetic parameters measured for the interaction of CEACAM1 with RSB02 affinity matured variants are shown in Table 14. All the humanized, affinity matured variants demonstrated affinity improvements compared to Parent (VH2 N56Q/Vκ4 S27(f)A).

He3/L20, He17/L1, He17/L16 variants demonstrate significantly faster on and off-rates compared with parent IgG.

TABLE 14
Multi-cycle kinetic data for RSB01 Parent (VH2 N56Q/Vκ4 S27(f)A)
and the twelve humanized affinity matured leads binding to CEACAM1 as determined
using the Biacore T200. The relative K_{D} compared to Parent (VH2 N56Q/Vκ4 S27(f)A)
was calculated by dividing the K_{D} of the Parent by that of the affinity
matured variants assayed on the same chip.

| | | | | | Relative |
|---|---|---|---|---|---|
| | | | | | K_{D} to |
| Variant | kₐ (1/Ms) | k_{d} (1/s) | K_{D} (M) | Chi² | Parent |
| Parent (VH2 N56Q/ | 8.13 × 10⁴ | 6.92 × 10⁻³ | 8.52 × 10⁻⁸ | 0.488 | 1.00 |
| Vk4 S27(f)A) | | | | | |
| He3/L20* | 3.06 × 10⁵ | 7.80 × 10⁻³ | 2.55 × 10⁻⁸ | 4.51 | 3.34 |
| He17/L1* | 3.02 × 10⁵ | 4.09 × 10⁻³ | 1.35 × 10⁻⁸ | 3.99 | 6.31 |
| Parent (VH2 N56Q/ | 8.18 × 10⁴ | 6.98 × 10⁻³ | 8.53 × 10⁻⁸ | 0.467 | 1.00 |
| Vk4 S27(f)A) | | | | | |
| VHp/L1 | 3.31 × 10⁴ | 4.13 × 10⁻³ | 1.25 × 10⁻⁷ | 0.0692 | 0.68 |
| He17/L16* | 3.08 × 10⁵ | 7.95 × 10⁻³ | 2.58 × 10⁻⁸ | 7.17 | 3.31 |
| Parent (VH2 N56Q/ | 4.20 × 10⁴ | 5.15 × 10⁻³ | 1.23 × 10⁻⁷ | 0.248 | 1.00 |
| Vk4 S27(f)A) | | | | | |
| He4/L16 | 3.75 × 10⁴ | 1.46 × 10⁻³ | 3.89 × 10⁻⁸ | 0.474 | 3.16 |
| He14/L1* | 1.08 × 10⁵ | 7.24 × 10⁻³ | 6.73 × 10⁻⁸ | 4.65 | 1.83 |
| Parent (VH2 N56Q/ | 4.02 × 10⁴ | 5.25 × 10⁻³ | 1.31 × 10⁻⁷ | 0.327 | 1.00 |
| Vk4 S27(f)A) | | | | | |
| He9/L4 #2 | 3.86 × 10⁴ | 1.30 × 10⁻³ | 3.37 × 10⁻⁸ | 0.685 | 3.89 |
| He9/L3 | 4.40 × 10⁴ | 1.39 × 10⁻³ | 3.16 × 10⁻⁸ | 1.16 | 4.15 |
| Parent (VH2 N56Q/ | 3.63 × 10⁴ | 6.75 × 10⁻³ | 1.86 × 10⁻⁷ | 0.0496 | 1.00 |
| Vk4 S27(f)A) | | | | | |
| VHp/L18 | 3.57 × 10⁴ | 3.62 × 10⁻³ | 1.01 × 10⁻⁷ | 0.0871 | 1.84 |
| He9/L17 | 3.67 × 10⁴ | 1.50 × 10⁻³ | 4.09 × 10⁻⁸ | 0.318 | 4.55 |
| Parent (VH2 N56Q/ | 5.48 × 10⁴ | 1.04 × 10⁻² | 1.91 × 10⁻⁷ | 0.0563 | 1.00 |
| Vk4 S27(f)A | | | | | |
| VHp/L4 #2 | 2.98 × 10⁴ | 4.37 × 10⁻³ | 1.47 × 10⁻⁷ | 0.0501 | 1.30 |
| He9/L6 | 3.81 × 10⁴ | 1.51 × 10⁻³ | 3.96 × 10⁻⁸ | 0.347 | 4.82 |

*highlights variants which show a poor 1:1 fit, resulting in less accurate KD and relative KD determination.

### Example 9: Specificity of Affinity Matured CEACAM1 Antibodies as Determined by Flow Cytometry

Binding selectivity of the selected antibody variants was assessed by flow cytometry using HeLa cells transfected with vectors expressing CEACAM1, CEACAM 3, CEACAM5, CEACAM6, or CEACAM 8, respectively. The resulting cells were used for flow cytometry experiments and were cultured at 37° C., 5.0% CO₂ in Dulbecco's modified Eagle's medium supplemented with 10% fetal bovine serum, penicillin (100 U/ml) and dihydrostreptomycin (100 g/ml). Cell lines were stained with the indicated antibodies followed by fluorochrome-conjugated monoclonal antibody specific for the indicated antibody isotypes such as human IgG4 or mouse IgG1 together with viable dye (DAPI). Data were acquired with a Cytoflex flow cytometer (Invitrogen) and analyzed with FlowJo software (TreeStar, V7.6.5 for Windows).

Binding specificities for selected CEACAM1 antibodies are shown in Table 15.

TABLE 15
Binding selectivity of the selected antibody variants
was assessed by flow cytometry using He1a cells
transfected with vectors expressing no CEACAM
(Neo), CEACAM1 (C1), CEACAM5 (C5), CEACAM6 (C6),
or CEACAM 8 (C8), respectively.

| VH | VL | Neo | C1 | C3 | C5 | C6 | C8 |
|---|---|---|---|---|---|---|---|
| He3 | 20 | | +++ | | | | |
| He4 | 16 | | +++ | | +++ | +++ | |
| He9 | 3 | | +++ | | +++ | +++ | |
| He9 | 6 | | +++ | | +++ | + | |
| He9 | 17 | | +++ | | +++ | ++ | |
| He9 | L4#2 | | +++ | | +++ | +++ | |
| He14 | 1 | | +++ | | | | |
| VH2 N56Q* | Vk4 S27(f)A | | +++ | | | | |
| 17 | 1 | | +++ | | + | + | |
| 17 | 16 | | +++ | | + | | |
| VH2 N56Q | 1 | | +++ | | | | |
| VH2 N56Q | 4#2 | | +++ | | + | | |
| VH2 N56Q | 18 | | +++ | | +++ | | |
| Col-1 | | | + | +++ | +++ | | |
| 9A6 | | | + | | | +++ | + |
| T84.1 | | | +++ | +++ | +++ | +++ | |
| T84.66 | | | + | | +++ | + | + |
| 80H3 | | +++ | + | | | | +++ |

Indicated is the relative amount of cells expressing the respective antigen that the indicated antibody bound to.
*indicates the parent antibody used for affinity maturation. Col-1 (CEACAM3/5), 9A6 (CEACAM6), T84.1 (CEACAM1, 3, 5), T84.66 (CEACAM5), and 80H3 (CEACAM 8) are control antibodies.

### Example 10: Specificity of Affinity Matured CEACAM1 Antibody He14/L1 as Determined by Surface Plasmon Resonance (SPR)

The binding specificity of antibody He14/L1 to hCEACAM1, hCEACAM3, hCEACAM5 and hCEACAM6 was shown by single cycle surface plasmon resonance (SPR).

To assess the binding of He14/L1 to hCEACAM1, hCEACAM3, hCEACAM5 and hCEACAM6, single cycle kinetic analysis was conducted. Kinetic experiments were performed at 25° C. on a Biacore T200 running Biacore T200 Control software V2.0.1 and Evaluation software V3.0 (Cytiva, Uppsala, Sweden). HBS-P+ (Cytiva, Uppsala, Sweden) was used as running buffer as well as for ligand and analyte dilutions. At the start of each cycle, antibodies were loaded onto a Protein A sensor chip (Cytiva, Uppsala, Sweden). IgGs were captured at a flow rate of 10 μl/min to a pre-determined immobilization level, taking into account the varying analyte molecular weights (100 RU for CEACAM1, 375 RU for hCEACAM3, 71 RU for hCEACAM5, and 150 RU for hCEACAM6). The surface was then allowed to stabilize. A three-point, two-fold dilution range from 70-280 nM of antigen in running buffer was used without regeneration between each concentration. The association phases were monitored for 80 sec for each of the three injections of increasing concentrations of antigen and a single dissociation phase was measured for 150 seconds following the last injection of antigen. A similar method was used for mouse D14HD11, an anti-pan hCEACAM control antibody. However, in this instance, a CM5 chip immobilized with an anti-mouse capture reagent (Cytiva, Uppsala, Sweden) was used, association was monitored for 275 sec and dissociation was monitored for 1800 seconds. The data was double-referenced and subtracted.

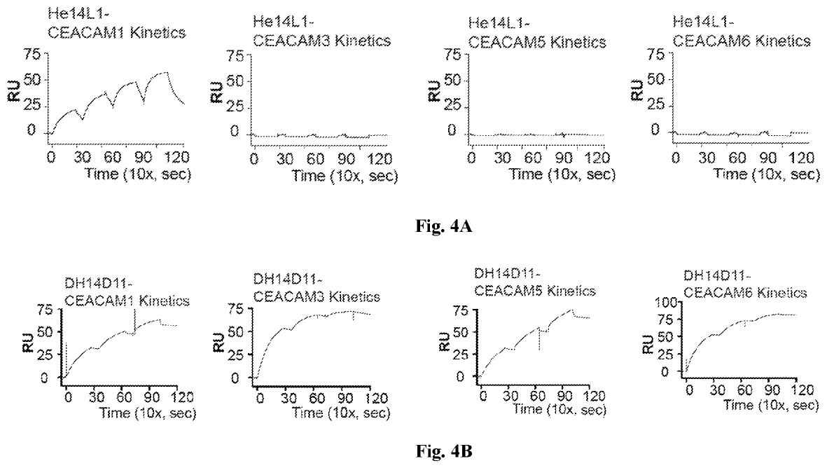

As shown in FIG. 4, antibody He14/L1 binds to hCEACAM1 with high specificity.

### Example 11: Crystal Structure of a CEACAM Antibody and CEACAM1

To precisely map the binding interface between CEACAM1 and humanized, affinity matured, and aglycosylated antibody He14/L1, the crystal structure of human CEACAM1 in complex with a He14/L1 Fab fragment was determined with atomic resolution (Table 16). The structure of the co-crystal has been deposited with the RCSB Protein Data Bank using PDB code 7N3W.

TABLE 16
Crystal information, data collection and refinement parameters
of the He14L1: human CEACAM1 structure (PDB ID 7N3W).

| | |
|---|---|
| Data collection | |
| | NYX (NSLS-II) |
| dₘᵢₙ (Å) | 1.84 |
| wavelength (Å) | 0.979 |
| No. of observations | 630632 |
| Multiplicity^{a} | 6.1 (6.3) |
| average (I)/(σ_{I})^{a} | 11.0 (1.2) |
| Completeness (%)a | 96.7 (91.8) |
| R_{merge} (%)^{a} | 0.1084 (1.364) |
| CC_{1/2}^{a} | 0.998 (0.494) |
| Structure refinement | |
| Bragg spacings (Å) | 45.36-1.85 |
| Space group | P2₁ |
| cell parameters: a, b, c | 55.14, 134.08, 86.00 and β = 92.83° |
| R/Rfree (%) | 16.2/20.8 |
| No. of unique reflections | 103,414 |
| No. of total atoms (non-H) | 9,348 |
| No. of protein atoms (non-H) | 8,456 |
| No. of waters | 16 |
| average B-factor (Å²) | 41.5 |
| rmsd bond length (Å) | 0.017 |
| rmsd bond angle (°) | 1.6 |
| Ramachandran favored/ | 97/2.8 |
| allowed (%) | |

^{a}Values in outermost shell are given in parentheses.

Tagless CEACAM1 was expressed in E. coli, refolded in an arginine-containing buffer, and purified. The He14/L1 Fab was prepared by digesting the provided antibody with immobilized papain resin and then purified with a 1 ml HiTrap MabSelect SuRe column (GE Healthcare) followed by gel filtration chromatography. Purified CEACAM1 and Fab were mixed in a 1:1 molar ratio prior to crystallization screening. Initial crystallization hits of the CEACAM1:He14/L1 Fab complex were identified and subsequently optimized. Diffraction quality crystals were grown at room temperature in a condition containing 20% PEG 8000 and 0.1 M HEPES pH 7.5. SDS-PAGE analysis and silver staining of a washed crystal reveal all expected components of the complex.

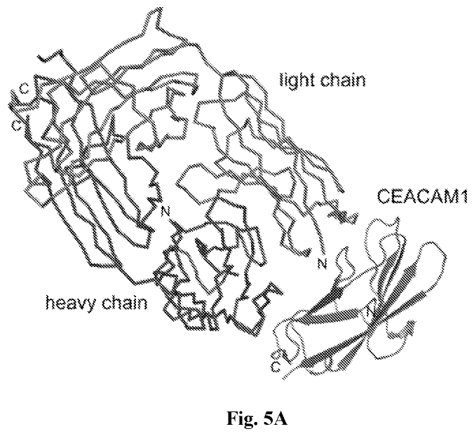

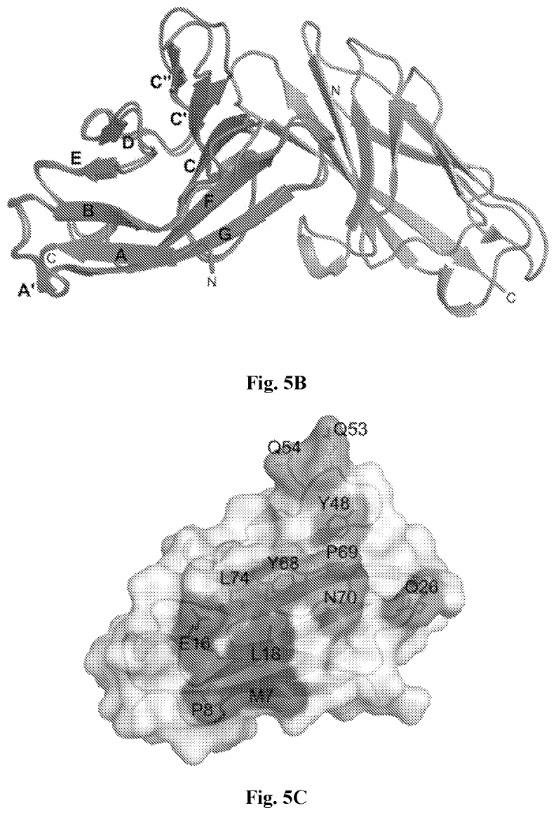

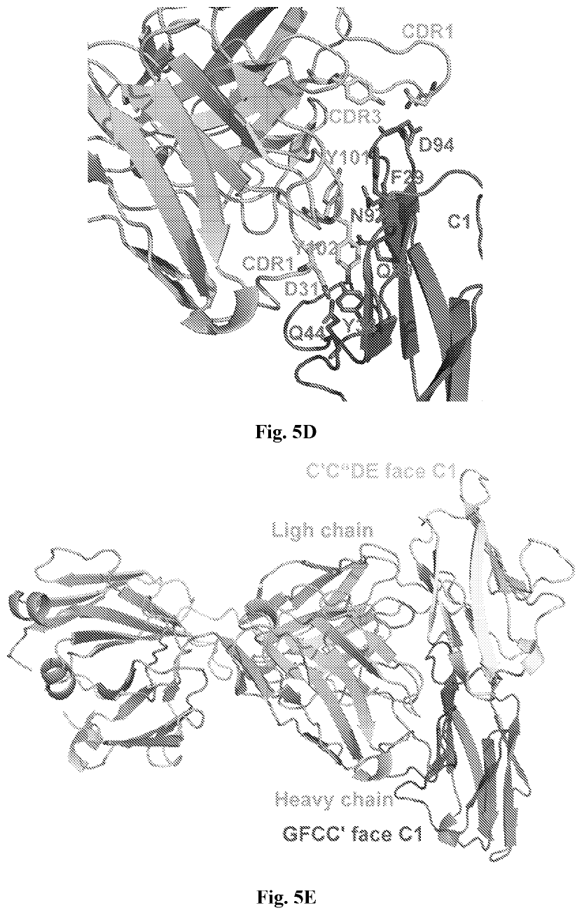

The structure of the CEACAM1:VH14/VL1 Fab complex was determined to 1.85 Å resolution. The complex crystallized in the primitive monoclinic spacegroup P21 with two copies of the complex seen in the asymmetric unit. In the structure coordinate file, chains A and L correspond to Fab light chains, chains B and H correspond to Fab heavy chains, and chains C and D correspond to molecules of CEACAM1. The structure of the complex (chains C, L, and H) is shown in FIG. 5A.

### GFCC′ Face Binding

The He14/L1Fab binds in a 1:1 stoichiometric ratio to CEACAM1 symmetry mate present in the crystal asymmetric unit through GFCC′ interface (FIG. 5D). In this interface, CEACAM1 GFCC′ residues specially F29, Q44, T56, Q89 and N97 contribute to tight binding with both heavy and light chain residues CDRs (Table 17) and CC′ and FG loops of CEACAM1 shows intense participation in complex formation. These GFCC′ face residues are also important for human CEACAM1 homodimer formation and its interactions with various ligands such as TIM-3, HopQ. Superimposition of CEACAM1 in the Fab complex with a protomer of CEACAM1 in the dimer yields a root-mean square deviation of 0.453 A over 81 aligned Ca positions. The GFCC′ binding interface has a higher shape complementarity score of 0.74 and this is a preferred face of binding.

### ABED Face Binding

The He14/L1Fab binds in a 1:1 stoichiometric ratio to CEACAM1 present in the crystal asymmetric unit (Table 18). Upon complex formation, the Fab appears to perturb the structure of CEACAM1. In particular, there are significant structural differences in β-strands C′ and C″, and the C′ to C″ loop, which are involved in self-association. In the complex, there are fewer intramolecular hydrogen bonding interactions within this perturbed region, leading to a noticeably shorter C″ strand with a shift in the orientation of C′ as residues seen to form the C″ strand in the dimer become part of an extended loop (FIG. 5B). Superimposition of CEACAM1 in the Fab complex with a protomer of CEACAM1 in the dimer yields a root-mean square deviation of 1.32 A over 81 aligned Ca positions. As shown, it appears that the perturbed loop interferes with dimerization and the associated surface is altered by Fab binding.

The epitope on CEACAM1 is shown in a molecular surface representation of CEACAM1 (FIG. 5C). In the He14/L1 Fab light chain, CDR1 interacts mainly with CEACAM1 residues in strand C′ and the C′ to C″ loop, while CDR3 interacts mainly with residues in strands D and E and the D to E loop. In the Fab heavy chain, CDR1 and CDR2 interact with strands A and B and the associated interconnecting loop. In addition, there may be a weak electrostatic interaction between side chains of the heavy chain Lys65 and CEACAM1 Glu (located 4.5 A apart, not drawn). The interacting surfaces have a shape complementarity of 0.58. The light chain CDR2 and the heavy chain CDR3 do not interact with CEACAM1.

The crystal structures of human CEACAM1 homodimer (PDB 4QXW) highlights importance of GFCC′ face in homodimer formation. Further, the ABED face crystal structure (PDB 2GK2) support the role of ABED face as a secondary minor interface in CEACAM1 oligomer formation. Both the GFCC′ and ABED face binding exist in the same crystal lattice of He14/LJ CEACAM1 complex crystal structure, indicating that He14/1L1 exhibits a unique dual binding mode to human CEACAM1 through the GFCC′ and ABED faces (FIG. 5E).

TABLE 17
He14/L1 Fab residues involved in binding
to CEACAM1's GFCC' face.

| | Antibody | Antibody | Hydrogen | Hydrophobic |
|---|---|---|---|---|
| CEACAM1 | Heavy chain | Light chain | bond | interaction |
| Residues | Residues | Residues | (Å) | (Å) |
| T56 (OG1) | D31 (OD2) | | 2.76* | |
| | (CDR1) | | | |
| Q44 (NE2) | Y102 (OH) | | 3.86* | |
| | (CDR3) | | | |
| Y48 (N) | Y102 (OH) | | 3.48* | |
| | (CDR3) | | | |
| L95 (O) | Y101 (OH) | | 2.73* | |
| | (CDR3) | | | |
| Y31 (O) | Y102 (OH) | | 3.89* | |
| | (CDR3) | | | |
| F29 | P104 (CDR3) | | | Aromatic- |
| | | | | Proline |
| F29 | Y33 (CDR1) | | 5.1 | |
| N97 | Y101 (CDR3) | | 4.5 | |
| Q89$ | Y101/Y102 | | | |
| | (CDR3) | | | |
| D94 (O) | | Y38 (OH) | 2.78* | |
| | | (CDR1) | | |
| D94 (OD2) | | N34 (ND2) | 2.91* | |
| | | (CDR1) | | |
| F29 | | Y98 (CDR3) | | Aromatic |

$The interaction site is located at the center of the CEACAM1/CEACAM1 binding interface.
The table describes the primary and secondary hydrogen-bonded and hydrophobic interactions and the residues involved.
*Primary hydrogen-bonded interactions.

TABLE 18
He14/L1 Fab residues involved in binding to ABED face. The primary and secondary
hydrogen (H) bonded interactions and residues involved are indicated.

| | Antibody | Antibody | H | |
|---|---|---|---|---|
| CEACAM1 | Heavy chain | Light chain | bond | |
| Residues | Residues | Residues | (Å) | Comment |
| M7 (O) (main | Q57 (NE2) | | 2.78* | CEACAM1 residue P8 is not |
| chain Oxygen) | (side chain) | | | directly involved in binding to the |
| | (CDR2) | | | antibody but provides the optimal |
| | | | | structural conformation for M7- |
| | | | | Q57 binding. |
| E16 (OE2) (side | Y33 (OH) | | 3.43* | |
| chain) | (hydroxyl) | | | |
| | (CDR1) | | | |
| Q26 (side chain) | | Q27 (side | 4.1 | CEACAM1 residue Q27 is not |
| | | chain) | | directly involved in binding to the |
| | | (CDR1) | | antibody but provides the optimal |
| | | | | structural conformation for Q26- |
| | | | | Q27 binding. |
| Y48 (hydroxyl) | | S32 (side | 4.5 | CEACAM1 residue A49 is not |
| | | chain) | | directly involved in binding to the |
| | | (CDR1) | | antibody but provides the optimal |
| | | | | structural conformation for Y48- |
| | | | | S32 binding. |
| Q53 (main chain | | A33 (main | 3.28 | CEACAM1 residue Q54 is not |
| oxygen) | | chain | | directly involved in binding to the |
| | | oxygen) | | antibody but provides the optimal |
| | | (CDR1) | | structural conformation for Q53- |
| | | | | A33 binding. |
| Y68 (OH) | | Y98 (OH) | 2.76* | CEACAM1 residue P69 is not |
| (hydroxyl) | | (hydroxyl) | | directly involved in binding to the |
| | | (CDR3) | | antibody but provides the optimal |
| | | | | structural conformation for Y68- |
| | | | | Y68 binding |
| N70 (ND2) (side | | P99 (O) | 2.8* | |
| chain) | | (main chain) | | |
| | | (CDR3) | | |

*Primary hydrogen-bonded interactions.

### Example 12: CEACAM1 Antibodies Block CEACAM1:TIM-3 Interactions

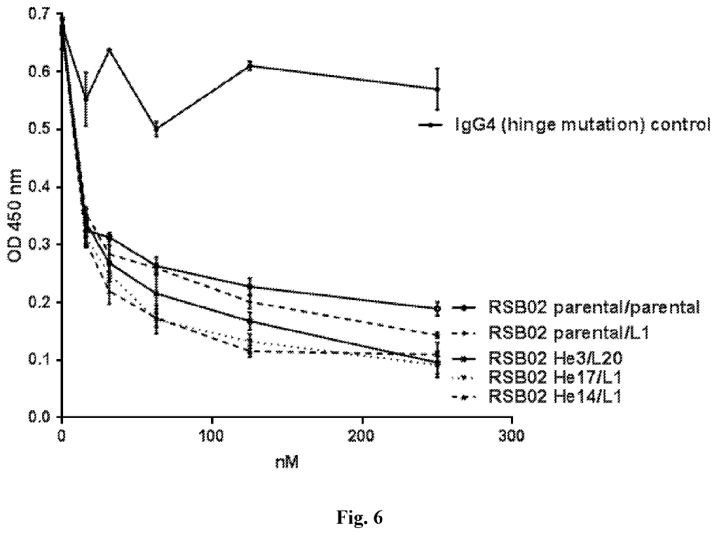

The ability of selected CEACAM1 antibodies to reduce the binding of CEACAM1 to TIM-3 was examined. CEACAM1/TIM-3 competition ELISA studies were done in triplicates to determine ability of selected antibodies (concentration range 0-250 nM) to inhibit human TIM-3 IgV domain tagless protein and human CEACAM1-GST protein binding. After incubation of 3 μg/ml hTIM-3 tagless protein overnight to coat the plate, the wells were washed and blocked with 2% BSA in Tris-buffered saline buffer containing 10 mM CaCl₂ (TBS-Ca²⁺ buffer). Blockade of hTIM-3 binding to hCEACAM1-GST protein was performed in the presence of antibodies at concentrations ranging from 0-250 nM using 4 μM hCEACAM1-GST tagged protein. Human IgG4 antibody was used as a control (0-250 nM). Goat polyclonal anti-GST-HSP antibody from Abcam (1:2000) was used for detection of human CEACAM1-GST protein and assays were developed by addition of TMB solution (Life technologies). OD values were read at 450 nm on a plate reader. Data was plotted in a Graphpad Prism and best-fit IC-50 values were determined. As shown in FIG. 6, the indicated CEACAM antibodies block CEACAM1:TIM-3 heterophilic interactions.

### Example 13: CEACAM1 Antibodies Reverse T Cell Functions in Tumor Dissociated Cells Derived from a Patient Resistant to Checkpoint Inhibitor Therapy

To assess the ability of CEACAM1 antibodies to reverse T cell exhaustion in patients that are resistant to treatment with checkpoint inhibitors such as PD-1/PD-L1 and CTLA-4 inhibitors, peripheral blood mononuclear cells (PBMCs) were isolated from a melanoma patient (CY123.1) with secondary resistance to Pembrolizumab (PD-1 inhibitor). The PBMCs were re-suspended and cultured under T cell stimulation conditions in 200 ml of complete medium (RPMI 1640, Lonza) supplemented with 10% fetal calf serum (FCS), 1% glutamine, 100 IU/ml penicillin, 100 μg/ml streptomycin (Life Technologies), 25 mM HEPES (Sigma-Aldrich) in triplicates using 96-well U bottom plates at a concentration of 0.5×10⁵ cells/ml with 100 LU/ml recombinant IL-2 (NIH) and soluble 1 mg/ml of CD3 (UCHT1, Abcam) in the presence of various concentrations (0.3-30 nM) of soluble chimeric (parental) or humanized and affinity-matured heavy (He3, He17, He14) and/or light ((L20, L1) chain variants or hIgG4 isotype control antibody. After culturing for 96 hr, cell proliferation was measured by assessing intracellular Ki67 expression in the CD3⁺CD8⁺ T cell fraction in triplicate.

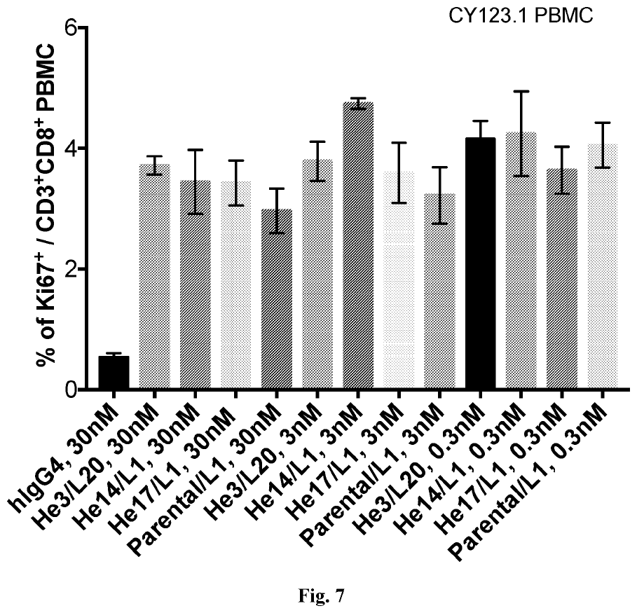

As shown in FIG. 7, CEACAM1 antibodies reverse T cell exhaustion in PD-1 resistant tumors.

### Example 14: CEACAM1 Antibody Induces Induction of B and T Cells in CD45-Positive Cells from Metastatic Melanomas

The effect of CEACAM1 antibody He14/L1 on CD45-positive cells from metastatic melanomas was examined.

Equal numbers of sorted CD45-positive cells from three metastatic melanomas (treatment naïve, n=1; treatment-resistant, n=2) were treated in vitro with He14/L1 (5 mg/ml) or human IgG4 isotype control (5 mg/ml) for 42 hours in medium at 37° C. and single-cell sequencing was performed. Briefly, the cells were processed using 10× Genomics Chromium Controller and the Chromium Single Cell 5′ Library & Gel Bead kit following the manufacturer's protocols. The samples were sequenced on the Illumina NovaSeq S2-100 apparatus. De-multiplexing, barcode processing, single-cell 5′ unique molecular identifier (UMI), transcript alignment and counting were performed using the Cell Ranger pipeline. The data were consecutively imported into R by Read10×( ) command function in Seurat for analyses. The number of cell counts in merged datasets and the number of UMI/transcripts per cell (nUMI) were plotted and evaluated and quality control metrics applied. The data sets were merged and the cells that are similar between groups established using canonical correlation analysis and the cells defined by applying a principal component analysis score with visualization of the metaclusters within a 2-dimensional UMAP. Cluster identification was based upon the feature genes. This identified the B cells within a metacluster (M2) and the T cells within four metaclusters (MO, M20, M23, M25) in the isotype (ISO) and He14/L1 samples.

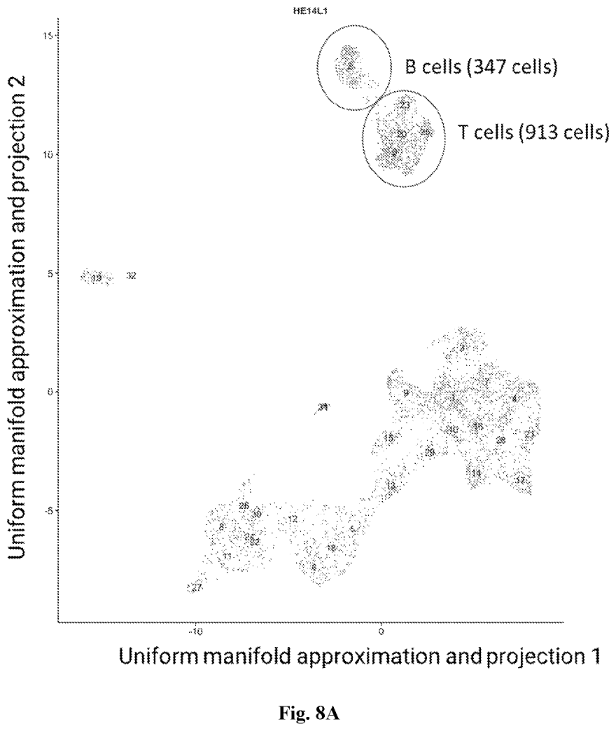

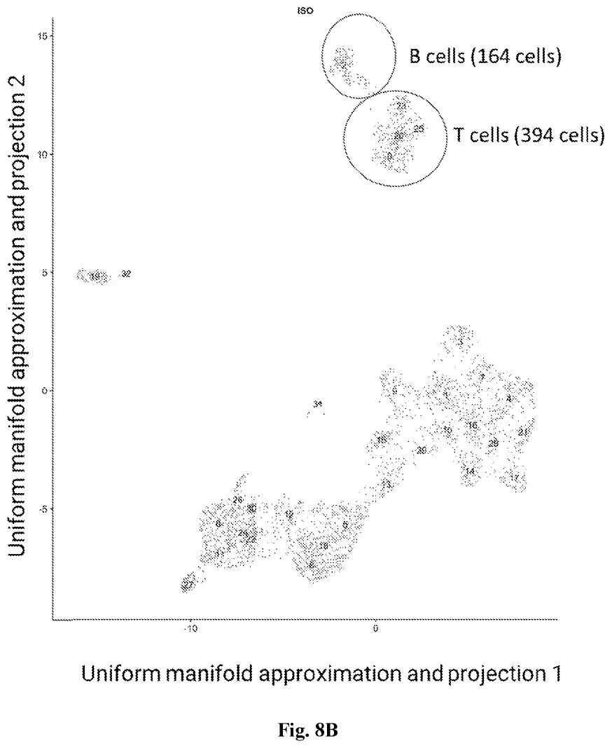

As shown in FIG. 8, treatment with antibody He14/L1 resulted in the expansion of B cells (from 164 to 347 cells) and T cells (from 394 to 913 cells) within 42 hours as compared to the control antibody, indicating that He14/L1 causes broad induction of B and T cells, responses that are important to anti-tumor activity.

### Example 15: CEACAM1 Expression on Cells from Patients Resistant to Treatment

CEACAM1 expression on tumor-associated B cells, tumor-associated monocytic cells, and T cells isolated from treatment-resistant and naïve patients was examined.

Peripheral blood mononuclear cells (PBMC) from healthy donors (HD, n=5), treatment naïve melanoma patients (PBMC-N, n=5) and treatment-resistant melanoma patients (PBMC-R, n=5), or mechanically dissociated metastatic tumors from treatment-naïve melanoma (Tumor-N, n=9) or treatment-resistant melanoma (Tumor-R, n=16) were stained with anti-human CEACAM1 monoclonal antibody (parental antibody for generation of He14/L1) labeled with Tb159 or a panel of heavy metal labeled antibodies for immune profiling (Maxpar® Direct™ Immune Profiling Assay™). The stained cells were analyzed on a CyTOF2 mass cytometer. Sample acquisition, file processing using R and mass cytometry data analysis using Cytobank to obtain comprehensive phenotyping by multiplexed single-cells was performed by standard methods. Cluster identification, characterization and regression (Citrus) analysis was performed to establish associations between markers and the clinical phenotypes. Based upon the markers as defined by manufacturer (Fludigm) cells consistent with B cell, monocytic cell, and CD8 T cell fractions were analyzed for the median levels of human CEACAM1 expression as detected by the Tb159 labeled monoclonal parental antibody in the treatment-naïve and -resistant samples in association with the metaclusters identified by Citrus analysis. Statistical analysis performed with the Kruskal-Wallis test followed by the Dunn's multiple comparison test. *, p, 0.05, **p<0.0

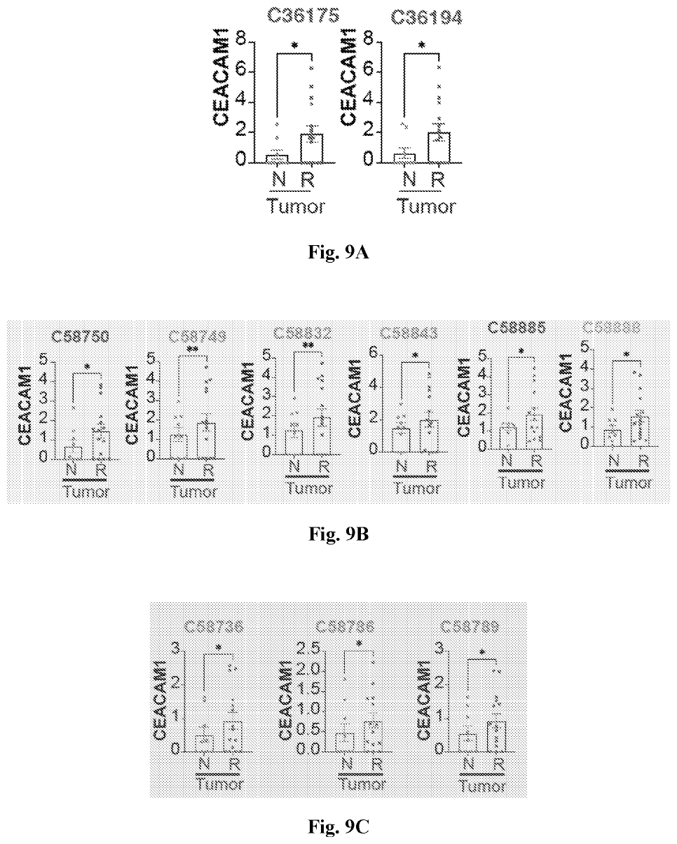

As shown in FIG. 9, CEACAM1 expression in metaclusters representing tumor-associated B cells, monocytic cells, and CD8⁺ T cells is increased in treatment-resistant tumor samples as compared to cells isolated from treatment-naïve tumor samples.

TABLE 19
Overview of sequences.

| | | |
|---|---|---|
| SEQ | Name for patent | |
| ID NO | application | Sequence |

Variable heavy chains

| | | |
|---|---|---|
| 10 | Chimeric | EVQLVESGGGFVKPGGSLKLSCAASGFSFSDYYLYWVRQTP |
| | parental variable | EKRLEWVATISVGGGNTSYPDSVKGRFTISRDNAKNNLYLQ |
| | heavy chain | MSSLKSEDTAMYYCTRGLYYGPAWFAYWGQGTLVTVSA |
| | (VH0) | |
| 11 | VH1 | EVQLLESGGGLVQPGGSLRLSCAASGFSFSDYYLYWVRQAP |
| | (humanized) | GKGLEWVATISVGGGNTSYPDSVKGRFTISRDNSKNTLYLQ |
| | | MNSLRAEDTAMYYCTRGLYYGPAWFAYWGQGTLVTVSS |
| 12 | VH2 | EVQLLESGGGLVQPGGSLRLSCAASGFSFSDYYLYWVRQAP |
| | (humanized) | GKGLEWVATISVGGGNTSYADSVKGRFTISRDNSKNTLYLQ |
| | | MNSLRAEDTAMYYCTRGLYYGPAWFAYWGQGTLVTVSS |
| 13 | VH1 | EVQLLESGGGLVQPGGSLRLSCAASGFSFSDYYLYWVRQAP |
| | (humanized, | GKGLEWVATISVGGGQTSYPDSVKGRFTISRDNSKNTLYLQ |
| | aglycosylated) | MNSLRAEDTAMYYCTRGLYYGPAWFAYWGQGTLVTVSS |
| 14 | VH2 | EVQLLESGGGLVQPGGSLRLSCAASGFSFSDYYLYWVRQAP |
| | (humanized, | GKGLEWVATISVGGGQTSYADSVKGRFTISRDNSKNTLYLQ |
| | aglycosylated) | MNSLRAEDTAMYYCTRGLYYGPAWFAYWGQGTLVTVSS |
| | Aglycosylation | |
| | mutation shown | |
| | in bold; CDRs | |
| | underlined | |
| 15 | He14 | EVQLLESGGGLVQPGGSLRLSCAASGFSFSDYYLYWVRQAP |
| | (humanized, | GKGLEWVATISVGGGQTSYADSVKGRFTISRDNSKNTLYLQ |
| | affinity matured, | MNSLRAEDTAMYYCTRGLYYGPSWVAYWGQGTLVTVSS |
| | aglycosylated) | |
| | Aglycosylation | |
| | mutation shown | |
| | in bold; CDRs | |
| | underlined | |
| 16 | He17 | EVQLLESGGGLVQPGGSLRLSCAASGFSFSDYYLYWVRQAP |
| | (humanized, | GKGLEWVATISVGGGQTSYADSVKGRFTISRDNSKNTLYLQ |
| | affinity matured, | MNSLRAEDTAMYYCTRARTYGPAWFAYWGQGTLVTVSS |
| | aglycosylated) | |
| | Aglycosylation | |
| | mutation shown | |
| | in bold; CDRs | |
| | underlined | |
| 17 | He3 (humanized, | EVQLLESGGGLVQPGGSLRLSCAASGFSFSDYYLYWVRQAP |
| | affinity matured, | GKGLEWVATISVGGGQTSYADSVKGRFTISRDNSKNTLYLQ |
| | aglycosylated) | MNSLRAEDTAMYYCTRALTYGPAWLAYWGQGTLVTVSS |
| | Aglycosylation | |
| | mutation shown | |
| | in bold; CDRs | |
| | underlined | |

Variable light chains

| | | |
|---|---|---|
| 18 | Chimeric | DIVMTQSPSSLAMSVGQKVTMSCKSSQSLLNSSNQKNYLAW |
| | parental variable | FQQTPGQSPKLLVYFASTRESGVPDRFIGSGSGTDFTLTIS |
| | light chain (Vκ0) | SVKAEDLADYFCQQHYSTPWTFGGGTKLEIK |
| 19 | Vκ4 | DIVMTQSPDSLAVSLGERATINCKSSQSLLNSSNQKNYLAW |
| | (humanized) | YQQKPGQPPKLLIYFASTRESGVPDRFSGSGSGTDFTLTIS |
| | | SLQAEDVAVYYCQQHYSTPWTFGGGTKVEIK |
| 20 | Vκ4 (humanized, | DIVMTQSPDSLAVSLGERATINCKSSQSLLNSANQKNYLAW |
| | aglycosylated) | YQQKPGQPPKLLIYFASTRESGVPDRFSGSGSGTDFTLTIS |
| | Aglycosylation | SLQAEDVAVYYCQQHYSTPWTFGGGTKVEIK |
| | mutation shown | |
| | in bold; CDRs | |
| | underlined | |
| 21 | L1 (humanized, | DIVMTQSPDSLAVSLGERATINCKSSQSLLNSANQKNYLAW |
| | affinity matured, | YQQKPGQPPKLLIYFASTRESGVPDRFSGSGSGTDFTLTIS |
| | aglycosylated) | SLQAEDVAVYYCQSHYPFYYTFGGGTKVEIK |
| | Aglycosylation | |
| | mutation shown | |
| | in bold; CDRs | |
| | underlined | |
| 22 | L20 (humanized, | DIVMTQSPDSLAVSLGERATINCKSSQSLLNSANQKNYLAW |
| | affinity matured, | YQQKPGQPPKLLIYFASTRESGVPDRFSGSGSGTDFTLTIS |
| | aglycosylated) | SLQAEDVAVYYCQSHFPYPLTFGGGTKVEIK |
| | Aglycosylation | |
| | mutation shown | |
| | in bold; CDRs | |
| | underlined | |

CDRs

| | | |
|---|---|---|
| 1 | He14 CDR1H = | DYYLY |
| | He17 CDR1H = | |
| | He3 CDR1H | |
| 2 | He14 CDR2H = | TISVGGGQTSYADSVKG |
| | He17 CDR2H = | |
| | He3 CDR2H | |
| 3 | He14 CDR3H | GLYYGPSWVAY |
| 4 | He17 CDR3H | ARTYGPAWFAY |
| 5 | He3 CDR3H | ALTYGPAWLAY |
| 6 | L1 CDR1L = | KSSQSLLNSANQKNYLA |
| | L20 CDR1L | |
| 7 | L1 CDR2L = | FASTRES |
| | L20 CDR2L | |
| 8 | L1 CDR3L | QSHYPFYYT |
| 9 | L20 CDR3L | QSHFPYPLT |

CEACAM1 sequences

| | | |
|---|---|---|
| 23 | CEACAM1 | MGHLSAPLHRVRVPWQGLLLTASLLTFWNPPTTAQLTTESM |
| | (NCBI | PFNVAEGKEVLLLVHNLPQQLFGYSWYKGERVDGNRQIVGY |
| | Reference | AIGTQQATPGPANSGRETIYPNASLLIQNVTQNDTGFYTLQ |
| | Sequence | VIKSDLVNEEATGQFHVYPELPKPSISSNNSNPVEDKDAVA |
| | NP_001703.2; | FTCEPETQDTTYLWWINNQSLPVSPRLQLSNGNRTLTLLSV |
| | UNIPROT ID | TRNDTGPYECEIQNPVSANRSDPVTLNVTYGPDTPTISPSD |
| | P13688) | TYYRPGANLSLSCYAASNPPAQYSWLINGTFQQSTQELFIP |
| | signal sequence: | NITVNNSGSYTCHANNSVTGCNRTTVKTIIVTELSPVVAKP |
| | residues 1-34 | QIKASKTTVTGDKDSVNLTCSTNDTGISIRWFFKNQSLPSS |
| | Ig-V N domain: | ERMKLSQGNTTLSINPVKREDAGTYWCEVFNPISKNQSDPI |
| | residues 35-142 | MLNVNYNALPQENGLSPGAIAGIVIGVVALVALIAVALACF |
| | | LHFGKTGRASDQRDLTEHKPSVSNHTQDHSNDPPNKMNEVT |
| | | YSTLNFEAQQPTQPTSASPSLTATEIIYSEVKKQ |
| 24 | mature form of | QLTTESMPFNVAEGKEVLLLVHNLPQQLFGYSWYKGERVDG |
| | CEACAM1 | NRQIVGYAIGTQQATPGPANSGRETIYPNASLLIQNVTQND |
| | (without signal | TGFYTLQVIKSDLVNEEATGQFHVYPELPKPSISSNNSNPV |
| | sequence) | EDKDAVAFTCEPETQDTTYLWWINNQSLPVSPRLQLSNGNR |
| | | TLTLLSVTRNDTGPYECEIQNPVSANRSDPVTLNVTYGPDT |
| | | PTISPSDTYYRPGANLSLSCYAASNPPAQYSWLINGTFQQS |
| | | TQELFIPNITVNNSGSYTCHANNSVTGCNRTTVKTIIVTEL |
| | | SPVVAKPQIKASKTTVTGDKDSVNLTCSINDTGISIRWFFK |
| | | NQSLPSSERMKLSQGNTTLSINPVKREDAGTYWCEVFNPIS |
| | | KNQSDPIMLNVNYNALPQENGLSPGAIAGIVIGVVALVALI |
| | | AVALACFLHFGKTGRASDQRDLTEHKPSVSNHTQDHSNDPP |
| | | NKMNEVTYSTLNFEAQQPTQPTSASPSLTATEIIYSEVKKQ |

Other sequences

| | | |
|---|---|---|
| 25 | VH CDR3 block | CTRGLYYGPAWFAYW |
| | for mutagenesis | |
| 26 | VL CDR 3 block | CQQHYSTPWTF |
| | for mutagenesis | |

While the foregoing written description of the invention enables one of ordinary skill to make and use what is considered presently to be the best mode thereof, those of ordinary skill will understand and appreciate the existence of variations, combinations, and equivalents of the specific embodiments, methods, and examples herein.

1. An antibody or antigen-binding fragment thereof that binds to CEACAM1, the antibody or antigen-binding fragment thereof comprising a heavy chain variable region and a light chain variable region;
wherein each of the heavy chain and the light chain variable regions comprises a CDR1, CDR2, and CDR3; and wherein:
a) the sequence of CDR1H comprises sequence DYYLY (SEQ ID NO:1);
b) the sequence of CDR2H comprises sequence TISVGGGQTSYADSVKG (SEQ ID NO:2);
c) the sequence of CDR3H comprises sequence GLYYGPSWVAY (SEQ ID NO:3), ARTYGPAWFAY (SEQ ID NO:4), or ALTYGPAWLAY (SEQ ID NO:5);
d) the sequence of CDR1L comprises sequence KSSQSLLNSANQKNYLA (SEQ ID NO:6);
e) the sequence of CDR2L comprises sequence FASTRES (SEQ ID NO:7); and
f) the sequence of CDR3L comprises sequence QSHYPFYYT (SEQ ID NO:8) or QSHFPYPLT (SEQ ID NO:9).

2. The antibody or antigen-binding fragment thereof according to claim 1, wherein:
a) the sequence of CDR3H comprises sequence GLYYGPSWVAY (SEQ ID NO:3); and
b) the sequence of CDR3L comprises sequence QSHYPFYYT (SEQ ID NO:8).

3. The antibody or antigen-binding fragment thereof according to claim 1, wherein the antibody or antigen-binding fragment thereof comprises a heavy chain variable region and a light chain variable region; wherein:
a) the sequence of the heavy chain variable region comprises a sequence that is at least 90% identical to a sequence selected from the group consisting of SEQ ID NOs:15-17; and
b) the sequence of the light chain variable region comprises a sequence that is at least 90% identical to SEQ ID NO:21 or SEQ ID NO:22.

4.-6. (canceled)

7. The antibody or antigen-binding fragment thereof according to claim 3, wherein:
a) the sequence of the heavy chain variable region comprises a sequence selected from the group consisting of SEQ ID NOs:15-17; and
b) the sequence of the light chain variable region comprises SEQ ID NO:21 or SEQ ID NO:22.

8. The antibody or antigen-binding fragment thereof according to claim 7, wherein:
a) the sequence of the heavy chain variable region comprises SEQ ID NO:15; and
b) the sequence of the light chain variable region comprises SEQ ID NO:21.

9. (canceled)

10. The antibody or antigen-binding fragment thereof according to claim 1, wherein the antibody or antigen-binding fragment thereof is a multispecific or a bispecific antibody or antigen-binding fragment thereof.

11. (canceled)

12. The antibody or antigen-binding fragment thereof according to claim 1, wherein the antibody or antigen-binding fragment thereof is an scFv, Fv, Fab′, Fab, F(ab′)₂, or diabody.

13-16. (canceled)

17. The antibody or antigen-binding fragment thereof according to claim 1, wherein the antibody or antigen-binding fragment thereof is conjugated to one or more of a cytotoxin, a fluorescent label, and an imaging agent.

18.-22. (canceled)

23. An isolated nucleic acid encoding the antibody or antigen-binding fragment thereof according to claim 1.

24. A vector comprising the nucleic acid according to claim 23.

25. An isolated cell comprising the vector according to claim 24.

26. (canceled)

27. A T-cell comprising a chimeric antigen receptor comprising the CDRs of the antibody or antigen-binding fragment thereof according to claim 1.

28. A pharmaceutical composition comprising the antibody or antigen-binding fragment thereof according to claim 1, and a pharmaceutically acceptable excipient.

29. A method of inhibiting binding of CEACAM1 to a member of the CEACAM family, the TIM family, or PD-1, the method comprising contacting CEACAM1 with the antibody or antigen-binding fragment thereof according to claim 1.

30.-34. (canceled)

35. A method of inhibiting binding of CEACAM1 to a bacterial adhesin, the method comprising contacting CEACAM1 with the antibody or antigen-binding fragment thereof according to claim 1.

36. (canceled)

37. A method of inhibiting binding of CEACAM1 to a Candida albicans, the method comprising contacting CEACAM1 with the antibody or antigen-binding fragment thereof according to claim 1.

38.-48. (canceled)

49. A method of treating cancer in a subject in need thereof, the method comprising administering to the subject the antibody or antigen-binding fragment thereof according to claim 1.

50. The method according to claim 49, wherein the cancer is melanoma, pancreatic cancer, thyroid cancer, lung cancer, colorectal cancer, squamous cancer, prostate cancer, breast cancer, bladder cancer, or gastric cancer.

51. (canceled)

52. A method of reducing tumor metastasis in a subject in need thereof, the method comprising administering to the subject the antibody or antigen-binding fragment thereof according to claim 1.

53-60. (canceled)

61. The method according to claim 49, the method further comprising administering to the subject a checkpoint inhibitor.

62. The method according to claim 61, wherein the checkpoint inhibitor is a CTLA-4, a PD-1, a PD-L1, and a PD-L2 inhibitor, or a TIM-3 inhibitor.

63. The method according to claim 49, the method further comprising administering one or more of an inhibitor of LAG3, TIGIT, LAP, Podoplanin, Protein C receptor, ICOS, GITR, CD226 and/or CD160.

64.-66. (canceled)

67. The method of treating a subject in need thereof, the method comprising administering to the subject the antibody or antigen-binding fragment thereof according to claim 1, wherein the subject has acquired resistance to therapy with a checkpoint inhibitor therapy.

68. The method according to claim 67, wherein the subject has acquired resistance to therapy with one or more of a PD-1 inhibitor, a PD-L1 inhibitor, or a CTLA-4 inhibitor.


SEQUENCE LISTING (47 sequences)

SEQ ID NO: 1 - protein, 5 aa, Mus musculus
DYYLY

SEQ ID NO: 2 - protein, 17 aa, synthetic construct
TISVGGGQTSYADSVKG

SEQ ID NO: 3 - protein, 11 aa, synthetic construct
GLYYGPSWVAY

SEQ ID NO: 4 - protein, 11 aa, synthetic construct
ARTYGPAWFAY

SEQ ID NO: 5 - protein, 11 aa, synthetic construct
ALTYGPAWLAY

SEQ ID NO: 6 - protein, 17 aa, synthetic construct
KSSQSLLNSANQKNYLA

SEQ ID NO: 7 - protein, 7 aa, Mus musculus
FASTRES

SEQ ID NO: 8 - protein, 9 aa, synthetic construct
QSHYPFYYT

SEQ ID NO: 9 - protein, 9 aa, synthetic construct
QSHFPYPLT

SEQ ID NO: 10 - protein, 120 aa, Mus musculus
EVQLVESGGGFVKPGGSLKLSCAASGFSFSDYYLYWVRQTPEKRLEWVATISVGGGNTSYPDSVKGRFTISRDNAKNNLYLQMSSLKSEDTAMYYCTRGLYYGPAWFAYWGQGTLVTVSA

SEQ ID NO: 11 - protein, 120 aa, synthetic construct
EVQLLESGGGLVQPGGSLRLSCAASGFSFSDYYLYWVRQAPGKGLEWVATISVGGGNTSYPDSVKGRFTISRDNSKNTLYLQMNSLRAEDTAMYYCTRGLYYGPAWFAYWGQGTLVTVSS

SEQ ID NO: 12 - protein, 120 aa, synthetic construct
EVQLLESGGGLVQPGGSLRLSCAASGFSFSDYYLYWVRQAPGKGLEWVATISVGGGNTSYADSVKGRFTISRDNSKNTLYLQMNSLRAEDTAMYYCTRGLYYGPAWFAYWGQGTLVTVSS

SEQ ID NO: 13 - protein, 120 aa, synthetic construct
EVQLLESGGGLVQPGGSLRLSCAASGFSFSDYYLYWVRQAPGKGLEWVATISVGGGQTSYPDSVKGRFTISRDNSKNTLYLQMNSLRAEDTAMYYCTRGLYYGPAWFAYWGQGTLVTVSS

SEQ ID NO: 14 - protein, 120 aa, synthetic construct
EVQLLESGGGLVQPGGSLRLSCAASGFSFSDYYLYWVRQAPGKGLEWVATISVGGGQTSYADSVKGRFTISRDNSKNTLYLQMNSLRAEDTAMYYCTRGLYYGPAWFAYWGQGTLVTVSS

SEQ ID NO: 15 - protein, 120 aa, synthetic construct
EVQLLESGGGLVQPGGSLRLSCAASGFSFSDYYLYWVRQAPGKGLEWVATISVGGGQTSYADSVKGRFTISRDNSKNTLYLQMNSLRAEDTAMYYCTRGLYYGPSWVAYWGQGTLVTVSS

SEQ ID NO: 16 - protein, 120 aa, synthetic construct
EVQLLESGGGLVQPGGSLRLSCAASGFSFSDYYLYWVRQAPGKGLEWVATISVGGGQTSYADSVKGRFTISRDNSKNTLYLQMNSLRAEDTAMYYCTRARTYGPAWFAYWGQGTLVTVSS

SEQ ID NO: 17 - protein, 120 aa, synthetic construct
EVQLLESGGGLVQPGGSLRLSCAASGFSFSDYYLYWVRQAPGKGLEWVATISVGGGQTSYADSVKGRFTISRDNSKNTLYLQMNSLRAEDTAMYYCTRALTYGPAWLAYWGQGTLVTVSS

SEQ ID NO: 18 - protein, 113 aa, Mus musculus
DIVMTQSPSSLAMSVGQKVTMSCKSSQSLLNSSNQKNYLAWFQQTPGQSPKLLVYFASTRESGVPDRFIGSGSGTDFTLTISSVKAEDLADYFCQQHYSTPWTFGGGTKLEIK

SEQ ID NO: 19 - protein, 113 aa, synthetic construct
DIVMTQSPDSLAVSLGERATINCKSSQSLLNSSNQKNYLAWYQQKPGQPPKLLIYFASTRESGVPDRFSGSGSGTDFTLTISSLQAEDVAVYYCQQHYSTPWTFGGGTKVEIK

SEQ ID NO: 20 - protein, 113 aa, synthetic construct
DIVMTQSPDSLAVSLGERATINCKSSQSLLNSANQKNYLAWYQQKPGQPPKLLIYFASTRESGVPDRFSGSGSGTDFTLTISSLQAEDVAVYYCQQHYSTPWTFGGGTKVEIK

SEQ ID NO: 21 - protein, 113 aa, synthetic construct
DIVMTQSPDSLAVSLGERATINCKSSQSLLNSANQKNYLAWYQQKPGQPPKLLIYFASTRESGVPDRFSGSGSGTDFTLTISSLQAEDVAVYYCQSHYPFYYTFGGGTKVEIK

SEQ ID NO: 22 - protein, 113 aa, synthetic construct
DIVMTQSPDSLAVSLGERATINCKSSQSLLNSANQKNYLAWYQQKPGQPPKLLIYFASTRESGVPDRFSGSGSGTDFTLTISSLQAEDVAVYYCQSHFPYPLTFGGGTKVEIK

SEQ ID NO: 23 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 24 - protein, 492 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 25 - protein, 15 aa, Mus musculus
CTRGLYYGPAWFAYW

SEQ ID NO: 26 - protein, 11 aa, Mus musculus [sequence omitted]

SEQ ID NO: 27 - protein, 14 aa, Mus musculus
CTRGLYYGPAWFAY

SEQ ID NO: 28 - protein, 14 aa, synthetic construct
CTRALTYGPAWLAY

SEQ ID NO: 29 - protein, 14 aa, synthetic construct
CTRALYYGPAWFAY

SEQ ID NO: 30 - protein, 14 aa, synthetic construct
CARARTYGPAWFAY

SEQ ID NO: 31 - protein, 14 aa, synthetic construct
CARAMYYGPAWFAY

SEQ ID NO: 32 - protein, 14 aa, synthetic construct
CTRARTYGPAWFAY

SEQ ID NO: 33 - protein, 14 aa, synthetic construct
CTRGLYYGPSWFAY

SEQ ID NO: 34 - protein, 14 aa, synthetic construct
CTRGLYYGPSWLAY

SEQ ID NO: 35 - protein, 14 aa, synthetic construct
CTRGLYYGPSWVAY

SEQ ID NO: 36 - protein, 9 aa, Mus musculus
QQHYSTPWT

SEQ ID NO: 37 - protein, 9 aa, synthetic construct
QSHYPFPYT

SEQ ID NO: 38 - protein, 9 aa, synthetic construct
QSHFPFPFT

SEQ ID NO: 39 - protein, 9 aa, synthetic construct
QSHFPYYYT

SEQ ID NO: 40 - protein, 9 aa, synthetic construct
QSHYPYPYT

SEQ ID NO: 41 - protein, 9 aa, synthetic construct
QSHYPFAFT

SEQ ID NO: 42 - protein, 9 aa, synthetic construct
QSHYPFSFT

SEQ ID NO: 43 - protein, 9 aa, synthetic construct
QSHYPYPFT

SEQ ID NO: 44 - protein, 9 aa, synthetic construct
QSHYPYPYT

SEQ ID NO: 45 - protein, 9 aa, synthetic construct
QSHFPYPFT

SEQ ID NO: 46 - protein, 9 aa, synthetic construct
QSHIYPNFT

SEQ ID NO: 47 - protein, 15 aa, synthetic construct
GGGGSGGGGSGGGGS